SEQUENCE LISTING (64 sequences)

SEQ ID NO: 1 - DNA, 26441 bp, synthetic construct
  misc_feature 1..26441: note = Non-native coronavirus
  source 1..26441: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactgccgccagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatctacatgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagtcagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgttcaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaa

SEQ ID NO: 2 - DNA, 26441 bp, synthetic construct
  misc_feature 1..26441: note = Non-native coronavirus
  source 1..26441: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactgccgccagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattatagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatctacatgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagtcagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagaagtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttttttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgttcaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaa

SEQ ID NO: 3 - DNA, 26441 bp, synthetic construct
  misc_feature 1..26441: note = Non-native coronavirus
  source 1..26441: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactgccgccagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatctacatgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagtcagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagaagtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttatacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgttcaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaa

SEQ ID NO: 4 - DNA, 26443 bp, synthetic construct
  misc_feature 1..26443: note = Non-native coronavirus
  source 1..26443: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactgccgccagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatctacatgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagtcagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagaagtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaacgttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaaatttttaaggtggtgtaccctgtggatgatcatcactttgaggtgatcctgcactatggcacactggtggtcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgttcaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattggaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaacgagtaactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaa

SEQ ID NO: 5 - DNA, 26441 bp, synthetic construct
  misc_feature 1..26441: note = Non-native coronavirus
  source 1..26441: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactgccgccagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatctacatgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagtcagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagaagtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgatgatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttatcttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgttcaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattggaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgagactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaa

SEQ ID NO: 6 - DNA, 26441 bp, synthetic construct
  misc_feature 1..26441: note = Non-native coronavirus
  source 1..26441: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactgccgccagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatctacatgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagtcagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaatgctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgttcaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaa

SEQ ID NO: 7 - DNA, 26441 bp, synthetic construct
  misc_feature 1..26441: note = Non-native coronavirus
  source 1..26441: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactgccgccagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagttttttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatctacatgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagtcagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagaagtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagataatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttatacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatcttacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgatggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgttcaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaa

SEQ ID NO: 8 - DNA, 26441 bp, synthetic construct
  misc_feature 1..26441: note = Non-native coronavirus
  source 1..26441: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactgccgccagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaatttagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatctacatgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagtcagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagaagtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttatacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttgtaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgttcaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaa

SEQ ID NO: 9 - DNA, 24384 bp, synthetic construct
  misc_feature 1..24384: note = Non-native coronavirus
  source 1..24384: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactgccgccagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtatagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtttagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatctacatgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagtcagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagaagtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagttggtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttatacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcccacaatcgacggttcatccggagttgttaatccagtaatggaaccaatttatgatgaaccgacgacgactactagcgtgcctttgtaagcacaagctgatgagtacgaacttatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgagactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaa

SEQ ID NO: 10 - DNA, 26441 bp, synthetic construct
  misc_feature 1..26441: note = Non-native coronavirus
  source 1..26441: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactgccgccagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacattgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatctacatgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaagttgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagtcagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagaagtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttatacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgttactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttatgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgttcaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaa

SEQ ID NO: 11 - DNA, 26441 bp, synthetic construct
  misc_feature 1..26441: note = Non-native coronavirus
  source 1..26441: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactgccgccagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatctacatgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagtcagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagaagtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagtcttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgttcaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaa

SEQ ID NO: 12 - DNA, 26441 bp, synthetic construct
  misc_feature 1..26441: note = Non-native coronavirus
  source 1..26441: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactgccgccagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatgtagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatctacatgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagtcagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagaagtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactaatcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagataatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttatacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttataccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatcttacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgttcaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaa

SEQ ID NO: 13 - DNA, 26441 bp, synthetic construct; identical to SEQ ID NO: 1
  misc_feature 1..26441: note = Non-native coronavirus
  source 1..26441: mol_type = other DNA

SEQ ID NO: 14 - DNA, 29882 bp, SARS-CoV-2
  source 1..29882: mol_type = genomic DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagtcagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactttttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgttcaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaa

SEQ ID NO: 15 - DNA, 13519 bp, synthetic construct
  misc_feature 1..13519: note = Synthetic nucleic acid molecule
  source 1..13519: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttagtaccgggcccgctctagtccggaatcagtcctgctcctcggccacgaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatggtggccctcctatagtgagtcgtattatactatgccgatatactatgccgatgattaattgtcaacacgtgctgcaggtccgaggttctagacgagtttactccctatcagtgatagagaacgatgtcgagtttactccctatcagtgatagagaacgtatgtcgagtttactccctatcagtgatagagaacgtatgtcgagtttactccctatcagtgatagagaacgtatgtcgagtttatccctatcagtgatagagaacgtatgtcgagtttactccctatcagtgatagagaacgtatgtcgaggtaggcgtgtacggtgggaggcctatataagcagagctcgtttagtgaaccgtcagatcgcaccggtccaccatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaacgcgtggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgctagcggatccataacttcgtatagtatacattatacgaagttatgccaccatgtctaggctggacaagagcaaagtcataaacggagctctggaattactcaatggtgtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgattttgaccttgacatgctccccgggtaaataacttcgtatagtatacattatacgaagttatggatccgcggccgcaaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatggccaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggctcgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgctggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagttcgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 16 - DNA, 26441 bp, synthetic construct
  misc_feature 1..26441: note = Non-native coronavirus
  source 1..26441: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttctttcggagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatctacatgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagtcagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgttcaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaa

SEQ ID NO: 17 - DNA, 26441 bp, synthetic construct
  misc_feature 1..26441: note = Non-native coronavirus
  source 1..26441: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtagtgagggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatctacatgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagtcagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgttcaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaa

SEQ ID NO: 18 - DNA, 37 bp, synthetic construct
  source 1..37: mol_type = other DNA
atatagaccggtccaccatgtctgataatggacccca

SEQ ID NO: 19 - DNA, 32 bp, synthetic construct
  source 1..32: mol_type = other DNA
atatagacgcgtttaggcctgagttgagtcag

SEQ ID NO: 20 - DNA, 17 bp, synthetic construct
  source 1..17: mol_type = other DNA
gtaaaacgacggccagt

SEQ ID NO: 21 - DNA, 37 bp, synthetic construct
  source 1..37: mol_type = other DNA
caaggttcttctttcggagaacggtaataaaggagct

SEQ ID NO: 22 - DNA, 37 bp, synthetic construct
  source 1..37: mol_type = other DNA
ttattaccgttctccgaaagaagaaccttgcggtaag

SEQ ID NO: 23 - DNA, 36 bp, synthetic construct
  source 1..36: mol_type = other DNA
taagaacggtagtgagggagctggtggccatagtta

SEQ ID NO: 24 - DNA, 36 bp, synthetic construct
  source 1..36: mol_type = other DNA
caccagctccctcactaccgttcttacgaagaagaa

SEQ ID NO: 25 - DNA, 40 bp, synthetic construct
  source 1..40: mol_type = other DNA
aaaactggaacactgccgccagcagtggtgttacccgtga

SEQ ID NO: 26 - DNA, 42 bp, synthetic construct
  source 1..42: mol_type = other DNA
gggtaacaccactgctggcggcagtgttccagttttcttgaa

SEQ ID NO: 27 - DNA, 20 bp, synthetic construct
  source 1..20: mol_type = other DNA
cacgagcagcctctgatgca

SEQ ID NO: 28 - DNA, 21 bp, synthetic construct
  source 1..21: mol_type = other DNA
ccctgtgggttttacacttaa

SEQ ID NO: 29 - DNA, 19 bp, synthetic construct
  source 1..19: mol_type = other DNA
acgattgtgcatcagctga

SEQ ID NO: 30 - DNA, 28 bp, synthetic construct
  source 1..28: mol_type = other DNA
ccgtctgcggtatgtggaaaggttatgg

SEQ ID NO: 31 - DNA, 22 bp, synthetic construct
  source 1..22: mol_type = other DNA
ccaaccaactttcgatctcttg

SEQ ID NO: 32 - DNA, 20 bp, synthetic construct
  source 1..20: mol_type = other DNA
ggacttggtccaggttgtag

SEQ ID NO: 33 - DNA, 27 bp, synthetic construct
  source 1..27: mol_type = other DNA
acgaacaatggtcttcacactcgaaga

SEQ ID NO: 34 - DNA, 27 bp, synthetic construct
  source 1..27: mol_type = other DNA
cgatctcttgtagatctgttctctaaa

SEQ ID NO: 35 - DNA, 20 bp, synthetic construct
  source 1..20: mol_type = other DNA
gcccagtcatagccgaatag

SEQ ID NO: 36 - DNA, 24 bp, synthetic construct
  source 1..24: mol_type = other DNA
acaagatggattgcacgcaggttc

SEQ ID NO: 37 - RNA, 106 nt, synthetic construct
  source 1..106: mol_type = other RNA
caggtaacaaaccaaccaactttcgatctcttgtagatctgttctctaaacgaacaatggtcttcacactcgaagatttcgttggggactggcgacagacagccgg

SEQ ID NO: 38 - RNA, 107 nt, synthetic construct
  source 1..107: mol_type = other RNA
caggtaacaaaccaaccaactttcgatctcttgtagatctgttctctaaacgaacttatggatttgtttatgagaatcttcacaattggaactgtaactttgaagca

SEQ ID NO: 39 - RNA, 107 nt, synthetic construct
  source 1..107: mol_type = other RNA
caggtaacaaaccaaccaactttcgatctcttgtagatctgttctctaaacgaacttatgattgaacaagatggattgcacgcaggttctccggccgcttgggtgga

SEQ ID NO: 40 - RNA, 108 nt, synthetic construct
  source 1..108: mol_type = other RNA
caggtaacaaaccaaccaactttcgatctcttgtagatctgttctctaaacgacgaacatgaaaattattcttttcttggcactgataacactcgctacttgtgagct

SEQ ID NO: 41 - RNA, 105 nt, synthetic construct
  source 1..105: mol_type = other RNA
caggtaacaaaccaaccaactttcgatctcttgtagatctgttctctaaacgaacatgaaatttcttgttttcttaggaatcatcacaactgtagctgcatttca

SEQ ID NO: 42 - RNA, 113 nt, synthetic construct
  source 1..113: mol_type = other RNA
caggtaacaaaccaaccaactttcgatctcttgtagatctgttctctaaacgaacaaactaaaatgtctgataatggaccccaaaatcagcgaaatgcaccccgcattacgtt

SEQ ID NO: 43 - DNA, 22 bp, synthetic construct
  source 1..22: mol_type = other DNA
ccaaccaactttcgatctcttg

SEQ ID NO: 44 - DNA, 20 bp, synthetic construct
  source 1..20: mol_type = other DNA
caacttctactaagccacaa

SEQ ID NO: 45 - DNA, 20 bp, synthetic construct
  source 1..20: mol_type = other DNA
agagctatgaattgcagaca

SEQ ID NO: 46 - DNA, 20 bp, synthetic construct
  source 1..20: mol_type = other DNA
cacgagcagcctctgatgca

SEQ ID NO: 47 - DNA, 20 bp, synthetic construct
  source 1..20: mol_type = other DNA
ctggtgagtttaaattggct

SEQ ID NO: 48 - DNA, 20 bp, synthetic construct
  source 1..20: mol_type = other DNA
tgtcaatgtcactaacaaga

SEQ ID NO: 49 - DNA, 20 bp, synthetic construct
  source 1..20: mol_type = other DNA
accgctgctttaggtgtttt

SEQ ID NO: 50 - DNA, 25 bp, synthetic construct
  source 1..25: mol_type = other DNA
ttgacacataagctgactgtagtaa

SEQ ID NO: 51 - DNA, 20 bp, synthetic construct
  source 1..20: mol_type = other DNA
atgttggtgatagtgcggaa

SEQ ID NO: 52 - DNA, 29 bp, synthetic construct
  source 1..29: mol_type = other DNA
gtgtgtaacagatgttaccaactgcacta

SEQ ID NO: 53 - DNA, 20 bp, synthetic construct
  source 1..20: mol_type = other DNA
actcaatatctcagatgagt

SEQ ID NO: 54 - DNA, 20 bp, synthetic construct
  source 1..20: mol_type = other DNA
tttcaagacttgtaaattgc

SEQ ID NO: 55 - DNA, 22 bp, synthetic construct
  source 1..22: mol_type = other DNA
ccaaccaactttcgatctcttg

SEQ ID NO: 56 - DNA, 20 bp, synthetic construct
  source 1..20: mol_type = other DNA
tcactccgttgatggttact

SEQ ID NO: 57 - DNA, 22 bp, synthetic construct
  source 1..22: mol_type = other DNA
cgatctcttgtagatctgttct

SEQ ID NO: 58 - DNA, 21 bp, synthetic construct
  source 1..21: mol_type = other DNA
aagaactcgtcaagaaggcga

SEQ ID NO: 59 - DNA, 540 bp, synthetic construct
  source 1..540: mol_type = other DNA
atggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactgccgccagcagtggtgttacccgtgaactcatgcgtgagcttaacggaggg

SEQ ID NO: 60 - DNA, 540 bp, SARS-CoV-2
  source 1..540: mol_type = genomic DNA
atggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggg

SEQ ID NO: 61 - DNA, 1500 bp, synthetic construct
  source 1..1500: mol_type = other DNA
aaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagtcagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagaagtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcag

SEQ ID NO: 62 - DNA, 1500 bp, SARS-CoV-2
  source 1..1500: mol_type = genomic DNA
aaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagtcagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcag

SEQ ID NO: 63 - DNA, 417 bp, synthetic construct
  source 1..417: mol_type = other DNA
gctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttatacttaaaaacacagtctgtaccgtctgcggtatgtggaaaggttatggctgtagttgtgatcaactccgcgaacccatgcttcag

SEQ ID NO: 64 - DNA, 417 bp, SARS-CoV-2
  source 1..417: mol_type = genomic DNA
gctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcag

(19) United States
(12) Patent Application Publication
(10) Pub. No.: US 2025/0250647 A1
(43) Pub. Date: Aug. 7, 2025

(54) STABLE CELL CLONES HARBORING REPLICATING SARS-COV-2 RNA

(71) Applicant: THE UNITED STATES OF AMERICA, as represented by the Secretary, Department of Health and Human, Silver Spring, MD (US)

(72) Inventors: Tony Tianyi Wang, Silver Spring, MD (US); Shufeng Liu, Silver Spring, MD (US)

(73) Assignee: THE UNITED STATES OF AMERICA, as represented by the Secretary, Department of Health and Human, Silver Spring, MD (US)

(21) Appl. No.: 18/704,230

(22) PCT Filed: Oct. 31, 2022
(86) PCT No.: PCT/US2022/078969

§ 371 Date: Apr. 24, 2024

Related U.S. Application Data
(60) Provisional application No. 63/275,251, filed on Nov. 3, 2021.

(51) Int. Cl. C12Q 1/70; C07K 14/005; C12Q 1/6897

(52) U.S. Cl. CPC C12Q 1/701; C07K 14/005; C12Q 1/6897; C12N 2770/20021; C12N 2770/20022; C12Q 2600/158

(57) ABSTRACT

Coronavirus genomes comprising non-native SARS-CoV-2 RNAs having genetically inactivated spike (S), envelope (E), and membrane (M) genes, and optionally a genetically inactivated nucleocapsid (NP) gene, are provided. Such coronavirus genomes further include a reporter gene and a marker gene, and substitutions within a non-structural protein 1 (Nsp1) gene (such as K164A and H165A). Also provided are cells containing the coronavirus genomes, for example, stable cell clones having the isolated non-native coronavirus genome autonomously replicating inside the cells. Also provided are methods of using such cells, for example, methods of identifying anti-viral compounds, such as quantitative high-throughput screening methods that can optionally be performed in a biosafety level 2 (BSL2) laboratory.

### CROSS-REFERENCE TO RELATED APPLICATION

This application claims priority to U.S. provisional Application No. 63/275,251, filed Nov. 3, 2021, herein incorporated by reference in its entirety.

### FIELD

This disclosure relates to isolated, non-native coronavirus genomes and stable cell clones containing the non-native coronavirus genomes for use in identifying anti-viral compounds.

### BACKGROUND

The single-stranded, positive-sense SARS-CoV-2 RNA genome is approximately 30 kb in length and comprises a short 5′ untranslated region (UTR), 13 open reading frames (ORFs), a 3′ UTR and a poly(A) tail downstream of the 3′UTR. Through discontinuous transcription events, the virus makes at least 9 canonical subgenomic RNAs (sgmRNA), which encode structural and accessory proteins (Kim et al., Cell 181, 914-921 e910 (2020)). The genomic RNA (gRNA) harbors two large ORFs, ORF1a and ORF1ab, which are initially translated into two polyproteins, pp1a and pp1ab, and subsequently processed by viral proteases to produce 16 non-structural proteins (Nsp) that form the viral replication complex and confer immune evasion (Rashid et al., Virus Res 296, 198350 (2021); Xia et al., Cell Rep 33, 108234 (2020); Lei et al., Nat Commun 11, 3810 (2020)).

Viral replication and translation machinery offer targets for antiviral drug development. The main protease Nsp5 and the viral RNA-dependent RNA polymerase Nsp12 are targets for antiviral discovery because they are responsible for cleavage of replicase polyproteins 1a and 1ab and for virus replication and transcription, respectively. A cell-based system that harbors the minimally essential SARS-CoV-2 replication and translation machinery without generating infectious virus could enable simultaneous screening of inhibitors of multiple viral proteins in a biosafety level 2 (BSL2) setting. For example, hepatitis C virus (HCV) replicon cell clones revolutionized the discovery of direct-acting antivirals (DAAs) for treatment of chronic HCV infection (Lohmann et al., Science 285, 110-113 (1999); Blight et al., Science 290, 1972-1974 (2000)). However, such a system is not yet available for SARS-CoV-2 due to intrinsic toxicity.

Replicons are subgenomic viral RNA molecules capable of autonomously replicating in cells. SARS-CoV-2 replicon systems that have been reported only allow transient expression of viral genes, i.e., do not allow persistent replication in cell lines due to intrinsic toxicity (Xia et al., Cell Rep 33, 108234 (2020); He et al., Proc Natl Acad Sci USA. 118 (15) e2025866118 (2021); Kotaki, et al., Sci Rep 11, 2229 (2021); Wang et al., Virol Sin, April 9:1-11 (2021)). The rapid loss of viral sgmRNA or a reporter gene, the inability to generate master and working cell banks for lot consistency, and the challenge to scale up for industrial processes, makes it impractical to apply transient replicon systems in high-throughput screening (HTS) of large compound libraries. Thus, robust, cell-based systems for genetic and functional analyses of SARS-CoV-2 replication and for the development of antiviral drugs are needed.

### SUMMARY

Provided herein are isolated non-native coronavirus genomes, cells expressing the genomes, as well as methods of using such cells (for example in methods of screening for anti-viral compounds, such as anti-SARS-CoV-2 compounds). In some embodiments, the isolated non-native coronavirus genomes include (i) genetically inactivated spike (S), envelope (E), and membrane (M) genes, and optionally also an inactivated (NP) gene; (ii) a reporter gene; (iii) a marker gene; and (iv) a non-structural protein 1 (Nsp1) gene encoding (a) R124S and K125E substitutions, (b) N128S and K129E substitutions, or (c) K164A and H165A substitutions. In some embodiments, the genetically inactivated S, E, and M genes, and optionally the genetically inactivated NP gene, include one or more inactivating nucleotide mutations, insertions, or deletions. In some embodiments, the genetically inactivated S, E, and M genes, and optionally the genetically inactivated NP gene, are deleted and replaced with another coding sequence, such as the reporter gene or the marker gene. In some embodiments, the genetically inactivated and M genes are deleted and replaced with a single coding sequence, such as the reporter gene or the marker gene.

In specific, non-limiting embodiments, the Nsp1 gene K164A substitution is encoded by guanine, cytosine, and cytosine (GCC) at nucleotides 490, 491, and 492, respectively of Nsp1 (SEQ ID NO: 59), corresponding to nucleotides 755, 756, and 757 of SEQ ID NO: 1, respectively, and the H165A substitution is encoded guanine, cytosine, and cytosine (GCC) at nucleotides 493, 494, and 495, respectively, of Nsp1 (SEQ ID NO: 59), corresponding to nucleotides 758, 759, and 760 of SEQ ID NO: 1, respectively. In other embodiments, the isolated non-native coronavirus genome includes a non-structural protein 4 (Nsp4) gene encoding a R401S substitution (e.g., SEQ ID NO: 61), a non-structural protein 10 (Nsp10) gene encoding a T111I substitution, or both substitutions (e.g., SEQ ID NO: 63).

In some embodiments of the disclosed isolated non-native coronavirus genomes, the marker gene is a selectable marker gene, such as an antibiotic resistance gene, such as an antibiotic resistance gene that confers resistance to neomycin, kanamycin, geneticin, ampicillin, or a combination thereof. In specific, non-limiting embodiments, the antibiotic resistance gene is a neomycin phosphotransferase gene. In some embodiments of the disclosed isolated non-native coronavirus genomes, the reporter gene encodes a fluorescent or bioluminescent protein, such as a luciferase or a nanoluciferase protein. In some examples, the marker gene replaces the native E and M sequences, the reporter gene replaces the native S sequence, or both. In some examples, the reporter gene replaces the native E and M sequences, the maker gene replaces the native S sequence, or both.

An isolated non-native coronavirus genome as disclosed herein can be a non-native betacoronavirus genome, such as a non-native SARS-CoV genome, a non-native SARS-CoV-2 genome, a non-native MERS-CoV genome, or another non-native betacoronavirus genome. In specific, non-limiting examples, an isolated nucleic acid molecule encoding the genome has at least 80%, at least 85%, at least 90%, at least 92%, at least 95%, at least 96%, at least 97%, at least 98%, at least 99%, or 100% sequence identity to SEQ ID NO: 1, 2, 3, 4, 5, 6, 7, 8, 9, 10, 11, 12, or 13. In one specific, non-limiting example, the isolated nucleic acid molecule encoding the non-native coronavirus genome consists of SEQ ID NO: 1, 2, 3, 4, 5, 6, 7, 8, 9, 10, 11, 12, or 13.

In some embodiments, the isolated non-native coronavirus genome is at least 20,000 kb, such as least 24,000 kb, such as 20,000-30,000 kb. In certain embodiments, the isolated non-native coronavirus genome is lyophilized. Also provided are compositions comprising an isolated non-native coronavirus genome disclosed herein and a pharmaceutically acceptable carrier.

In some examples, the disclosed non-native coronavirus genome is a DNA molecule. In some examples, the disclosed non-native coronavirus genome is an RNA molecule.

Also provided are isolated host cells including an isolated non-native coronavirus genome as disclosed herein. In some embodiments, the isolated non-native coronavirus genome is introduced into the cell using electroporation, liposome-mediated transfection, non-liposomal transfection, dendrimer-based transfection, particle bombardment, or microinjection. The disclosed isolated host cell can be a mammalian cell, such as a baby hamster kidney (BHK) cell, such as a BHK-21 cell (e.g., the cell deposited as American Type Culture Collection (ATCC) #CCL-10). In specific, non-limiting examples, the isolated host cell is the cell deposited as ATCC #. In some embodiments, the disclosed isolated host cell is a stable cell clone. In some embodiments, the isolated non-native coronavirus genome autonomously replicates in the host cell. Also provided are compositions that include isolated host cells as disclosed herein, and optionally a culture medium, DMSO, or both.

Also provided are methods of identifying anti-viral compounds, such as an anti-SARS-Cov-2 compound. The disclosed methods can include contacting an isolated host cell as disclosed herein with one or more compounds, determining a level of expression of the reporter gene in the contacted cells, and comparing the level of expression of the reporter gene in the contacted cells to a control. In such methods, reduced expression of the reporter gene in the contacted cells relative to the control indicates that the compound is an anti-viral compound. The disclosed methods can further include determining an IC₅₀ value for the one or more compounds. In some embodiments, the method is a quantitative high-throughput screening method. In some embodiments, the method further includes selecting compounds that reduced expression of the reporter gene in the contacted cells relative to the control.

In the disclosed methods, the coronavirus can be a betacoronavirus, such as a SARS-Cov, SARS-Cov-2, or MERS-CoV. In some embodiments of the disclosed methods, the method is performed in a biosafety level 2 (BSL2) laboratory.

Also provided are kits that include one or more disclosed isolated non-native coronavirus genomes, one or more disclosed isolated host cells, and one or more of an antibiotic, transfection reagents, and culture media.

The foregoing and other objects and features of the disclosure will become more apparent from the following detailed description, which proceeds with reference to the accompanying figures.

### BRIEF DESCRIPTION OF THE DRAWINGS

FIG. 1A is a schematic overview showing organization of a native SARS-CoV-2 RNA genome (top), and how this genome can be modified to generate a modified SARS-CoV-2 RNA genome that can stably replicate in cells (bottom). (Top) shows the genome organization of native SARS-CoV-2 (SEQ ID NO: 14). Leader sequence is shown in red on the left, and transcriptional regulatory sequences within the leader sequence (TRS-L) and within the body (TRS-B) are highlighted in green on the left. (middle) shows the design of SARS-CoV-2-Rep-NanoLuc-Neo (e.g., SEQ ID NO: 1). For example, the modified SARS-CoV-2 RNA genome can include genetically inactivated S and M genes, for example by replacing S with a reporter (e.g., NanoLuc), and E and M with a marker (e.g., NeoR) for selecting cells containing the modified SARS-CoV-2 RNA genome. (Bottom) shows the Nsp1 mutations introduced to obtain three more replicons (examples in SEQ ID NOs: 1-13, 16 and 17). For example, the modified SARS-CoV-2 RNA genome can further include mutations in NSP1, such as (a) R124S and K125E substitutions (e.g., SEQ ID NO: 16), (b) N128S and K129E substitutions (e.g., SEQ ID NO: 17), or (c) K164A and H165A (e.g., SEQ ID NOS: 1-13) substitutions.

FIG. 1B is an illustration of Nsp1 binding to the small ribosomal subunit (PDB code: 7K5I). Nsp1 (orange) binds close to the mRNA entry site and contacts uS3 (green) from the ribosomal 40S head as well as uS5 (blue) and h18 of the 18S rRNA (charcoal gray) of the 40S body. The fragment of rRNA not close to Nsp1 is shown as transparent.

FIG. 1C shows an enlarged view of the Nsp1 binding area. Interacting residues are shown in the stick representation and are highlighted in red.

FIG. 1D shows calculated free energy changes (AAG) for various mutations in Nsp1. Positive values indicate unfavorable mutations for the binding between Nsp1 and rRNA.

FIG. 1E shows BHK21-NP^{Dox-ON} cells transiently transfected with Rep-NanoLuc-Neo-Nsp1^{R124s/K125E} RNA (SEQ ID NO: 16), Rep-NanoLuc-Neo-Nsp1^{N12S/K129E} RNA (SEQ ID NO: 17), or Rep-NanoLuc-Neo-Nsp1^{K164A/H165A} RNA (SEQ ID NO: 1). Nano luciferase was measured at indicated time points post-transfection.

FIG. 1F shows an illustration of the MD system where Nsp-1 and rRNA (fragment) complex in a 0.15 M NaCl electrolyte. The equilibrated structure was used for the FEP calculations of the bound state.

FIG. 1G shows an illustration of the MD system where there is only Nsp-1 (no RNA) in a 0.15 M NaCl electrolyte. The equilibrated structure was used for the FEP calculations of the free state.

FIGS. 2A-2D show characterization of replicon cells harboring BHK21-NP^{Dox-ON} Rep-NanoLuc-Neo-Nsp1^{K164A/H165A}. Nano luciferase in BHK21-NP^{Dox-ON} replicon cells was measured at given time points following G418 withdrawal (FIG. 2A). RNA was also extracted at indicated time points and quantified by RT-qPCRs targeting ORFIab (gRNA in FIG. 2A), or sgmNeoR and sgmNanoLuc (FIG. 2B). FIG. 2C shows western blot analyses of the SARS-CoV-2 proteins from six representative stable replicon clones. The presence of NP and Nsp1 protein in cell lysates was confirmed. FIG. 2D shows sequence coverage of gRNA and sgmRNA species in Pool #1 and Pool #2 replicon cells as well as in each of the 12 stable clones.

FIGS. 3A-3B show the results of screening of a 273-compound library containing virtually identified candidates (see Table 5) in replicon cells (Pool #1, Rep-NanoLuc-Neo-Nsp1^{K164A/H165A} replicon cells) as described in Example 1. Ten compounds (including Remdesivir) displaying more than 50% inhibition were denoted in black or colored solid circles (FIG. 3A). FIG. 3B shows the molecular structure of Darapladib, Genz-123346, and JNJ-5207852.

FIG. 4A shows a clonal response to the 3CL protease inhibitor GC376. The half maximal inhibitory concentration (IC₅₀) of GC376 was determined on six stable replicon clones (#3, 5, 7, 9, 11, 13) (red). The effect of GC376 on cell viability (in grey) was simultaneously determined using the Cell Titer-Glo assay.

FIG. 4B shows measurement of nanoluciferase from parent BHK-21 or 12 stable replicon clones after 20 passages. The results are presented as relative light units (RLU) per 1,000 cells because of the differential growth rate of the clones.

FIGS. 5A-5D show replication kinetics of SARS-CoV-2-Rep-NanoLuc-Neo in different cell lines: Vero E6 (FIG. 5A), A549 (FIG. 5B), Huh7.5.1 (FIG. 5C) and BHK-21 cells (FIG. 5D) were electroporated with replicon RNA. Nano luciferase was measured at indicated time points post-electroporation.

FIG. 5E shows generation of BHK21 stable cells that express NP in a doxycycline-inducible manner. Cells were induced with 0.5 μg/ml doxycycline and lysed at 48 h post induction for western blotting with anti-NP and anti-actin antibodies. Numbers on the left refer to the positions of marker proteins in kilodalton (kDa).

FIG. 6 shows nanoluciferase kinetics of Rep-NanoLuc-Neo-Nsp1^{K164A/H65A} in Huh7.5.1 cells. Electroporated cells were lysed at indicated time points post-transfection for nanoluciferase quantification.

FIGS. 7A-7B show detection of viral RNA and proteins in replicon cells. FIG. 7A shows an RT-PCR analysis of viral RNA from replicon cells. The corresponding primer pairs are shown in the table on the right (from top to bottom, SEQ ID NOs: 43-58). The lengths of DNA fragments are indicated in the table. FIG. 7B shows detection of Nsp1 (green) and NP (red) in stable cell clones harboring Rep-NanoLuc-Neo-Nsp1^{K164A/H165A}.

FIGS. 8A-8M show detection of replicon RNA in stable cell clones. Sequence coverages of the gRNA in each individual stable cell clone as well as in Pool #1 cells are shown. Clone #9 (SEQ ID NO: 9) has a truncation in the NP region.

FIG. 9A shows morphological characterization of stable replicon clones with brightfield images of the 12 clones as well as the BHK-21-NPDox-ON cells as the negative control. Cell layers were not flat; hence certain cells in the field were off focus.

FIG. 9B shows characterization of the stable replicon clones by immunofluorescence images where red correlates to dsRNA stained by rJ2 anti-ds-RNA antibody. Cell layers were not flat; hence certain cells in the field were off focus. BHK-21-NPDox-ON cells shown for negative control.

FIG. 10A-10D shows quality control analysis of sequencing reads. The average Phred quality score remained high (>20) across all position and for all the samples and for both R1 (FIG. 10A) and R2 (FIG. 10B) ends of the paired-end reads. All samples produced more than 10M reads (FIG. 10C) while maintaining a small percentage of low-quality reads (FIG. 10D)

FIG. 11 shows an exemplary replicon map of SEQ ID NO: 1.

FIG. 12 shows an alignment of the wildtype (WT) Nsp1 gene from SARS-CoV-2 MN985325.1 (SEQ ID NO: 60) with the synthetic mutant gene Nsp1^{K164A/H165A} (SEQ ID NO: 61).

### SEQUENCE LISTING

The nucleic and amino acid sequences provided herein are shown using standard letter abbreviations for nucleotide bases, and one letter code for amino acids, in compliance with 37 C.F.R. 1.831-1.835 (87 Fed. Reg. 30806). Only one strand of each nucleic acid sequence is shown, but the complementary strand is understood as included by any reference to the displayed strand. The Sequence Listing is submitted as an Extensible Markup Language (.xml) file in the form of the file named “9531-107170-02 ST26 Sequence Listing.xml”, which was created on Oct. 24, 2022, and is 506,671 bytes, which is incorporated by reference herein.

Although DNA sequences are shown in the sequence listing, the corresponding RNAs are encompassed by this disclosure, by replacing “t” in the DNA sequence with “u”.

SEQ ID NO: 1 is an exemplary SARS-CoV-2-Rep-NanoLuc-Neo-Nsp1^{K164A/H165A}. The Nsp1 gene is located at nucleotides 266-805 of this sequence.

SEQ ID NO: 2 is SARS-CoV-2-Rep-NanoLuc-Neo-Nsp1^{K164A/H165A} of stable cell clone 2.

SEQ ID NO: 3 is SARS-CoV-2-Rep-NanoLuc-Neo-Nsp1^{K164A/H165A} of stable cell clone 3.

SEQ ID NO: 4 is SARS-CoV-2-Rep-NanoLuc-Neo-Nsp1^{K164A/H165A} of stable cell clone 4.

SEQ ID NO: 5 is SARS-CoV-2-Rep-NanoLuc-Neo-Nsp1^{K164A/H165A} of stable cell clone 5.

SEQ ID NO: 6 is SARS-CoV-2-Rep-NanoLuc-Neo-Nsp1^{K164A/H165A} of stable cell clone 6.

SEQ ID NO: 7 is SARS-CoV-2-Rep-NanoLuc-Neo-Nsp1^{K164A/H165A} of stable cell clone 7.

SEQ ID NO: 8 is SARS-CoV-2-Rep-NanoLuc-Neo-Nsp1^{K164A/H165A} of stable cell clone 8.

SEQ ID NO: 9 is SARS-CoV-2-Rep-NanoLuc-Neo-Nsp1^{K164A/H165A} of stable cell clone 9.

SEQ ID NO: 10 is SARS-CoV-2-Rep-NanoLuc-Neo-Nsp1^{K164A/H165A} of stable cell clone 10.

SEQ ID NO: 11 is SARS-CoV-2-Rep-NanoLuc-Neo-Nsp1^{K164A/H165A} of stable cell clone 11.

SEQ ID NO: 12 is SARS-CoV-2-Rep-NanoLuc-Neo-Nsp1^{K164A/H165A} of stable cell clone 12.

SEQ ID NO: 13 is SARS-CoV-2-Rep-NanoLuc-Neo-Nsp1^{K164A/H165A} of stable cell clone 13.

SEQ ID NO: 14 is an exemplary native SARS-Cov-2 genome sequence, SARS-CoV-2/human/USA/WA-CDC-WA1/2020 (GenBank Accession No. MN985325.1), which can be used to generate a disclosed modified SARS-CoV-2 RNA replicon. The native Nsp1 gene is located at nucleotides 266-805 of this sequence.

SEQ ID NO: 15 is the sequence of an NP^{Dox-ON} construct expressed in BHK-21 cells.

SEQ ID NO: 16 is SARS-CoV-2-Rep-NanoLuc-Neo-Nsp1^{R124S/K125E}

SEQ ID NO: 17 is SARS-CoV-2-Rep-NanoLuc-Neo-Nsp1^{N128S/K129E}

SEQ ID NO: 18 is an exemplary forward primer sequence for subcloning NP cDNA into a plasmid.

SEQ ID NO: 19 is an exemplary reverse primer sequence for subcloning NP cDNA into a plasmid.

SEQ ID NO: 20 is an exemplary M13 forward primer sequence for constructing plasmids.

SEQ ID NO: 21 is an exemplary forward primer sequence for constructing a plasmid which can introduce a R124S/K125E Nsp1 mutation into a SARS-CoV-2-Rep-NanoLuc-Neo replicon.

SEQ ID NO: 22 is an exemplary reverse primer sequence for constructing a plasmid which can introduce a R124S/K125E Nsp1 mutation into a SARS-CoV-2-Rep-NanoLuc-Neo replicon.

SEQ ID NO: 23 is an exemplary forward primer sequence for constructing a plasmid which can introduce a N128S/K129E Nsp1 mutation into a SARS-CoV-2-Rep-NanoLuc-Neo replicon.

SEQ ID NO: 24 is an exemplary reverse primer sequence for constructing a plasmid which can introduce a N128S/K129E Nsp1 mutation into a SARS-CoV-2-Rep-NanoLuc-Neo replicon.

SEQ ID NO: 25 is an exemplary forward primer sequence for constructing a plasmid which can introduce a K164A/H165A Nsp1 mutation into a SARS-CoV-2-Rep-NanoLuc-Neo replicon.

SEQ ID NO: 26 is an exemplary reverse primer sequence for constructing a plasmid which can introduce a K164A/H165A Nsp1 mutation into a SARS-CoV-2-Rep-NanoLuc-Neo replicon.

SEQ ID NO: 27 is an exemplary NheI reverse primer sequence for constructing plasmids.

SEQ ID NO: 28 is an exemplary ORF1ab forward primer sequence for quantifying viral RNA by reverse-transcription qPCR.

SEQ ID NO: 29 is an exemplary ORF1ab reverse primer sequence for quantifying viral RNA by reverse-transcription qPCR.

SEQ ID NO: 30 is an exemplary ORF1ab probe sequence for quantifying viral RNA by reverse-transcription qPCR.

SEQ ID NO: 31 is an exemplary NanoLuc gene subgenomic mRNA forward primer sequence for quantifying viral RNA by reverse-transcription qPCR.

SEQ ID NO: 32 is an exemplary NanoLuc gene subgenomic mRNA reverse primer sequence for quantifying viral RNA by reverse-transcription qPCR.

SEQ ID NO: 33 is an exemplary NanoLuc gene subgenomic mRNA probe sequence useful for quantifying viral RNA by reverse-transcription qPCR.

SEQ ID NO: 34 is an exemplary Neomycin phosphotransferase gene subgenomic mRNA forward primer sequence for quantifying viral RNA by reverse-transcription qPCR.

SEQ ID NO: 35 is an exemplary Neomycin phosphotransferase gene subgenomic mRNA reverse primer sequence for quantifying viral RNA by reverse-transcription qPCR.

SEQ ID NO: 36 is an exemplary Neomycin phosphotransferase gene subgenomic mRNA probe sequence for quantifying viral RNA by reverse-transcription qPCR.

SEQ ID NO: 37 is sgmNanoluc mRNA sequence.

SEQ ID NO: 38 is sgmORF3a mRNA sequence.

SEQ ID NO: 39 is sgmNeoR mRNA sequence.

SEQ ID NO: 40 is sgmORF7 mRNA sequence.

SEQ ID NO: 41 is sgmORF8 mRNA sequence.

SEQ ID NO: 42 is sgmNP mRNA sequence.

SEQ ID NO: 43 is exemplary primer 32f for the RT-PCR analysis of viral RNA.

SEQ ID NO: 44 is exemplary primer 434r for the RT-PCR analysis of viral RNA.

SEQ ID NO: 45 is exemplary primer 1000f for the RT-PCR analysis of viral RNA.

SEQ ID NO: 46 is exemplary primer 1892r for the RT-PCR analysis of viral RNA.

SEQ ID NO: 47 is exemplary primer 3000f for the RT-PCR analysis of viral RNA.

SEQ ID NO: 48 is exemplary primer 4072r for the RT-PCR analysis of viral RNA.

SEQ ID NO: 49 is exemplary primer 7000f for the RT-PCR analysis of viral RNA.

SEQ ID NO: 50 is exemplary primer 7965r for the RT-PCR analysis of viral RNA.

SEQ ID NO: 51 is exemplary primer 8000f for the RT-PCR analysis of viral RNA.

SEQ ID NO: 52 is exemplary primer 8932r for the RT-PCR analysis of viral RNA.

SEQ ID NO: 53 is exemplary primer 17000f for the RT-PCR analysis of viral RNA.

SEQ ID NO: 54 is exemplary primer 17990r for the RT-PCR analysis of viral RNA.

SEQ ID NO: 55 is exemplary primer sgNanof for the RT-PCR analysis of viral RNA.

SEQ ID NO: 56 is exemplary primer Nanor for the RT-PCR analysis of viral RNA.

SEQ ID NO: 57 is exemplary primer sgEf for the RT-PCR analysis of viral RNA.

SEQ ID NO: 58 is exemplary primer Neor for the RT-PCR analysis of viral RNA.

SEQ ID NO: 59 is an exemplary nucleotide sequence encoding Nsp1^{K164A/H165A}.

SEQ ID NO: 60 is an exemplary wildtype Nsp1 nucleotide sequence from exemplary native SARS-Cov-2 genome sequence, SARS-CoV-2/human/USA/WA-CDC-WA1/2020 (GenBank Accession No. MN985325.1).

SEQ ID NO: 61 is an exemplary nucleotide sequence encoding Nsp4^{R401S}.

SEQ ID NO: 62 is an exemplary wildtype Nsp4 nucleotide sequence from exemplary native SARS-Cov-2 genome sequence, SARS-CoV-2/human/USA/WA-CDC-WA1/2020 (GenBank Accession No. MN985325.1).

SEQ ID NO: 63 is an exemplary nucleotide sequence encoding Nsp10^{T111I}.

SEQ IN NO: 64 is an exemplary wildtype Nsp10 nucleotide sequence from exemplary native SARS-Cov-2 genome sequence, SARS-CoV-2/human/USA/WA-CDC-WA1/2020 (GenBank Accession No. MN985325.1).

### DETAILED DESCRIPTION

Unless otherwise noted, technical terms are used according to conventional usage. Definitions of common terms in molecular biology may be found in Benjamin Lewin, Genes X, published by Jones & Bartlett Publishers, 2009; and Meyers et al. (eds.), The Encyclopedia of Cell Biology and Molecular Medicine, published by Wiley-VCH in 16 volumes, 2008; and other similar references.

As used herein, the singular forms “a,” “an,” and “the,” refer to both the singular as well as plural, unless the context clearly indicates otherwise. For example, the term “a cell” includes single or plural cells and can be considered equivalent to the phrase “at least one cell.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. To facilitate review of the various embodiments, the following explanations of terms are provided:

About: Unless context indicated otherwise, “about” refers to plus or minus 5% of a reference value. For example, “about” 100 refers to 95 to 105.

Amino acid substitution: The replacement of one amino acid in a polypeptide (such as a coronavirus protein, such as a SARS-CoV-2 protein, such as an Nsp1 protein) with a different amino acid, such as replacement of a lysine with an alanine. In some examples, such a replacement is achieved by altering the coding sequence at the appropriate codon.

Anti-viral compound: An agent that reduces or inhibits viral replication and/or viral infection, such as SARS-CoV-2 replication and/or infection in a mammalian cell or subject. Some anti-viral agents target specific viruses (such as SARS-CoV-2), while a broad-spectrum anti-viral is effective against a wide range of viruses. On the basis of their target, exemplary antiviral compounds can be classified as follows: (1) entry blockers, which interfere with the attachment and penetration of the virus in the host cell; (2) nucleoside/nucleoside analogues and nonnucleoside analogues, which interfere with nucleic acid synthesis by blocking viral DNA polymerase or the retrotranscriptase in the case of RNA viruses (identified as NRTI (nucleos(t)ide retrotranscriptase inhibitors) and NNRTIs (nonnucleoside retrotranscriptase inhibitors), respectively); (3) IFNs, which inhibit protein synthesis necessary for viral replication; and (4) protease inhibitors, which interfere with the maturation of the virus and its infectivity.

Half-maximal inhibitory concentration (IC50) is an exemplary measure of drug (such as anti-viral compound) efficacy. An IC50 value indicates how much of a compound is needed to inhibit a biological process (such as transcription of a SARS-CoV-2 gene) by half (50%), thus providing a measure of potency of a drug for a given use.

Host Cell: A cell that has been genetically altered, or is capable of being genetically altered, by introduction of an exogenous polynucleotide, such as a recombinant plasmid or vector, or a non-native SARS-CoV-2 replicon provided herein. Typically, a host cell is a cell in which an exogenous polynucleotide can be propagated and expressed. The cell may be prokaryotic or eukaryotic. For example, the host cell may be a mammalian cell, including a baby hamster kidney cell, such as a BHK-21 cell. “Host cell” also includes a stable colony of cells, for example, a colony of BHK-21 cells. Thus, “contacting a host cell” and “incubating a host cell” include contacting a stable colony of host cells or incubating a stable colony of host cells. The term also includes any progeny of the subject host cell. A host cell encompasses material inside the outermost cell membrane, the outermost cell membrane itself and material fused or attached to the outermost cell membrane. In the case of a cell having a cell wall, the outermost cell membrane is the cell wall. Thus, the phase “within a host cell” includes material inside the outermost cell membrane, the outermost cell membrane itself and material fused or attached to the outermost cell membrane.

Conservative variants: “Conservative” amino acid substitutions are those substitutions that do not substantially affect or decrease a function of a protein (such as a SARS-CoV-2 protein). The term conservative variation also includes the use of a substituted amino acid in place of an unsubstituted parent amino acid. Furthermore, deletions or additions which alter, add or delete a single amino acid or a small percentage of amino acids (for instance less than 5%, in some embodiments less than 1%) in an encoded sequence are conservative variations where the alterations result in the substitution of an amino acid with a chemically similar amino acid.

The following six groups are examples of amino acids that are considered to be conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

In some examples, non-conservative substitutions alter an activity or function of a coronavirus protein, such as a SARS-CoV-2 protein, such as the ability to stably and/or autonomously replicate in a host cell, or the ability to cause cytotoxicity in a host cell. For instance, if an amino acid residue is essential for a function of the protein, even an otherwise conservative substitution may disrupt that activity. Thus, a conservative substitution does not alter the basic function of a protein of interest.

Contacting: Placement in direct physical association; includes both in solid and liquid form, which can take place either in vivo or in vitro. Contacting includes contact between one molecule and another molecule, for example between an antiviral compound and a cell, such as a stable cell clone harboring an isolated non-native coronavirus genome disclosed herein.

Control: A reference standard. In some embodiments, the control is a negative control sample, such as an untreated cell, such as an untreated cell containing a non-native coronavirus genome provided herein. In other embodiments, the control is a positive control sample, such as a cell (such as a host cell containing a non-native coronavirus genome provided herein) treated with a molecule having a known activity, such as a known antiviral compound that inhibits replication of a coronavirus. In still other embodiments, the control is a historical control or standard reference value or range of values (such as a previously tested control sample).

A difference between a test sample and a control can be an increase or conversely a decrease. The difference can be a qualitative difference or a quantitative difference, for example a statistically significant difference. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%.

Coronavirus (CoV): A large family of positive-sense, single-stranded RNA viruses that can infect humans and non-human animals. Coronaviruses have been organized into four groups: alphacoronaviruses (α-CoVs), betacoronaviruses (β-CoVs), gammacoronaviruses (γ-CoVs), and deltacoronaviruses (Δ-CoVs). Non-limiting examples of betacoronaviruses include SARS-CoV-2, Middle East respiratory syndrome coronavirus (MERS-CoV), Severe Acute Respiratory Syndrome coronavirus (SARS-CoV), Human coronavirus HKU1 (HKU1-CoV), Human coronavirus OC43 (OC43-CoV), Murine Hepatitis Virus (MHV-CoV), Bat SARS-like coronavirus WIV1 (WIV1-CoV), and Human coronavirus HKU9 (HKU9-CoV). Non-limiting examples of alphacoronaviruses include human coronavirus 229E (229E-CoV), human coronavirus NL63 (NL63-CoV), porcine epidemic diarrhea virus (PEDV), and Transmissible gastroenteritis coronavirus (TGEV). A non-limiting example of a deltacoronavirus is the Swine Delta Coronavirus (SDCV).

Coronaviruses get their name from the crown-like spikes on their surface. The viral envelope is comprised of a lipid bilayer containing the viral membrane (M), envelope (E) and spike (S) proteins. Most coronaviruses cause mild to moderate upper respiratory tract illness, such as the common cold. However, three coronaviruses have emerged that can cause more serious illness and death: severe acute respiratory syndrome coronavirus (SARS-CoV), SARS-CoV-2, and Middle East respiratory syndrome coronavirus (MERS-CoV). Other coronaviruses that infect humans include human coronavirus HKU1 (HKU1-CoV), human coronavirus OC43 (OC43-CoV), human coronavirus 229E (229E-CoV), and human coronavirus NL63 (NL63-CoV).

A coronavirus genome may be non-native, such as a non-native SARS-CoV-2 genome. A non-native coronavirus genome is genetically modified from a corresponding wild-type (native) coronavirus genome. For example, a non-native SARS-CoV-2 genome may include additional genes not present in a corresponding wild-type SARS-CoV-2 genome, and/or may include genetically inactivated SARS-CoV-2 genes, such as genetically inactivated SARS-CoV-2 spike (S), envelope (E), and/or membrane (M) genes, which may be replaced with a reporter gene and/or a marker gene, and can further include a Nsp1 gene encoding (a) R124S and K125E substitutions, (b) N128S and K129E substitutions, or (c) K164A and H165A substitutions. A coronavirus genome, such as a non-native coronavirus genome, may replicate autonomously inside a cell. In some examples, a non-native SARS-CoV-2 genome is a variant of SARS-CoV-2 (such as: alpha (B.1.1.7 and Q lineages); beta (B.1.351 and descendent lineages); delta (B.1.617.2 and AY lineages); gamma (P.1 and descendent lineages); epsilon (B.1.427 and B.1.429); eta (B.1.525); iota (B.1.526); kappa (B.1.617.1); 1.617.3; mu (B.1.621, B.1.621.1); zeta (P.2); and omicron (such as original lineage: B.1.1.529 and lineages: BA.2, BA.4, BA.5, BQ.1, BQ.1.1, BA.4.6, and BF.7)), and includes genetically inactivated SARS-CoV-2 spike (S), envelope (E), and membrane (M) genes, which may be replaced with a reporter gene and/or a marker gene, and can further include a Nsp1 gene encoding (a) R124S and K125E substitutions, (b) N128S and K129E substitutions, or (c) K164A and H165A substitutions, and may optionally include a genetically inactivated NP.

COVID-19: A contagious disease caused by severe acute respiratory syndrome coronavirus 2 (SARS-CoV-2). Symptoms of COVID-19 are variable, but often include fever, cough, fatigue, breathing difficulties, and loss of smell and taste. Symptoms can begin one to fourteen days after exposure to the virus. Around one in five infected individuals do not develop any symptoms. While most people have mild symptoms, some people develop acute respiratory distress syndrome (ARDS). ARDS can be precipitated by cytokine storms, multi-organ failure, septic shock, and blood clots. Longer-term damage to organs (in particular, the lungs and heart) has been observed. A significant number of patients recover from the acute phase of the disease but continue to experience a range of effects—known as long COVID—for months afterwards. These effects include severe fatigue, memory loss and other cognitive issues, low-grade fever, muscle weakness, and breathlessness.

Exogenous: The term “exogenous” as used herein with reference to nucleic acid and a particular cell refers to any nucleic acid that does not originate from that particular cell as found in nature. Thus, a non-naturally-occurring nucleic acid (such as a non-native SARS-CoV-2 genome) is considered to be exogenous to a cell once introduced into the cell. A nucleic acid that is naturally-occurring also can be exogenous to a particular cell. For example, an entire chromosome isolated from cell X is an exogenous nucleic acid with respect to cell Y once that chromosome is introduced into cell Y, even if X and Y are the same cell type.

Expression: Transcription or translation of a nucleic acid sequence. For example, an encoding nucleic acid sequence (such as a gene) can be expressed when its DNA is transcribed into RNA or an RNA fragment, which in some examples is processed to become mRNA. An encoding nucleic acid sequence (such as a gene) may also be expressed when its mRNA is translated into an amino acid sequence, such as a protein or a protein fragment. In a particular example, a heterologous gene is expressed when it is transcribed into an RNA. In another example, a heterologous gene is expressed when its RNA is translated into an amino acid sequence. Regulation of expression can include controls on transcription, translation, RNA transport and processing, degradation of intermediary molecules such as mRNA, or through activation, inactivation, compartmentalization or degradation of specific protein molecules after they are produced.

Genetic inactivation or down-regulation: When used in reference to the expression of a nucleic acid molecule, such as a gene, refers to any process which results in a decrease in production of a gene product. A gene product can be RNA (such as mRNA, rRNA, tRNA, and structural RNA) or protein. Therefore, gene down-regulation or deactivation includes processes that decrease transcription of a gene or translation of mRNA.

For example, a mutation, such as a substitution, partial or complete deletion, insertion, or other variation, can be made to a gene sequence that significantly reduces (and in some cases eliminates) production of the gene product or renders the gene product substantially or completely non-functional. For example, a genetic inactivation of a gene encoding a coronavirus E protein, such as a SARS-CoV-2 E protein, results in the virus having a non-functional or non-detectable E protein. Genetic inactivation is also referred to herein as “functional deletion”.

Isolated: A biological component (such as a nucleic acid molecule, protein, virus, or cell) that has been substantially separated, produced apart from, or purified away from other biological components in the cell of the organism (or in the organism) in which the component occurs, such as other chromosomal and extra-chromosomal DNA and RNA, and proteins. Thus, isolated nucleic acid molecules, viruses, and proteins include nucleic acid molecules, viruses, and proteins purified by standard purification methods. Similarly, an isolated host cell (or populations of cells) includes cells purified by standard purification methods from the organism or tissue in which they typically reside. The term also embraces nucleic acids and proteins prepared by recombinant expression in a host cell, as well as chemically synthesized nucleic acids and proteins. An isolated nucleic acid molecule, virus, protein, or host cell, such as a non-native SARS-CoV-2 genome provided herein or host cell containing such, can be at least 50%, at least 60%, at least 70%, at least 80%, at least 90%, at least 95%, at least 96%, at least 97%, at least 98%, at least 99%, at least 99.9%, or at least 99.99% pure.

Lyophilized: Lyophilization (also known as freeze drying) is a process by which water is removed from a material (such as a nucleic acid molecule or a composition comprising a nucleic acid molecule, such as a non-native SARS-CoV-2 genome) after it is frozen and placed under a vacuum, allowing the ice to change directly from solid to vapor without passing through a liquid phase. The lyophilization process can consist of three separate processes: freezing, primary drying (sublimation), and secondary drying (desorption). Lyophilization is commonly used to preserve perishable materials, such as nucleic acids, such as nucleic acid molecules encoding the disclosed isolated, non-native coronavirus genomes, to extend shelf life or make the material more convenient for transport.

Marker: A marker gene as used herein, such as a selectable marker, is a gene, which when introduced into a cell, confers a trait suitable for artificial selection of cells exhibiting the trait. Positive markers are selectable markers that confer selective advantage to the host cell, such as antibiotic resistance. An antibiotic resistance gene (the selectable marker gene) produces a protein that provides cells expressing the protein with resistance to a particular antibiotic. An antibiotic resistance gene may confer resistance to neomycin (such as a neomycin phosphotransferase gene), kanamycin, geneticin, ampicillin, or another antibiotic. Exemplary selectable marker genes include Neo (confers resistance to geneticin), bsd (confers resistance to blasticidin), hygB d (confers resistance to hygromycin B), pac (confers resistance to puromycin), and Sh bla (confers resistance to zeocin). Any of such can be present in a non-native coronavirus genome disclosed herein, for example in place of E and M genes, or the S gene. For example, a non-native coronavirus genome as disclosed herein can include a selectable marker, such as an antibiotic resistance gene (such as a neoR gene encoding the neomycin phosphotransferase enzyme), for selection of cells successfully transfected with a non-native coronavirus genome provided herein. In this example, cells treated (selected) using the antibiotic (such as neomycin or G418, an analog of neomycin sulfate) survive treatment if they express the antibiotic resistance gene.

Negative (or counterselectable) markers are selectable markers that eliminate or inhibit growth of the host cell upon selection, while positive and negative selectable markers can serve as both a positive and a negative marker by conferring an advantage to the host cell under one condition, and inhibiting growth under a different condition.

Nucleic acid molecule: A deoxyribonucleotide or ribonucleotide polymer or combination thereof including without limitation, DNA or RNA, such as cDNA, genomic DNA, subgenomic DNA (sgDNA), mRNA, rRNA, tNRA, and synthetic (such as chemically synthesized) DNA or RNA. The nucleic acid can be double stranded (ds) or single stranded (ss). Where single stranded, the nucleic acid can be the sense strand or the antisense strand. Nucleic acids can include natural nucleotides (such as A, T/U, C, and G), and can include analogs of natural nucleotides, such as labeled nucleotides.

“cDNA” refers to a DNA that is complementary or identical to an mRNA, in either single stranded or double stranded form.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence.

Pharmaceutically acceptable carriers: The pharmaceutically acceptable carriers of use are conventional. Remington's Pharmaceutical Sciences, by E. W. Martin, Mack Publishing Co., Easton, PA, 19th Edition, 1995, describes compositions and formulations suitable for pharmaceutical compositions, which include a non-native coronavirus genome or a cell containing the non-native coronavirus genome.

Examples of fluid carriers include pharmaceutically and physiologically acceptable fluids such as water, physiological saline, balanced salt solutions, aqueous dextrose, glycerol or the like as a vehicle. Examples of solid carriers include pharmaceutical grades of mannitol, lactose, starch, or magnesium stearate.

In addition to biologically neutral carriers, pharmaceutical compositions which include a non-native coronavirus genome or a cell containing the non-native coronavirus genome can contain minor amounts of non-toxic auxiliary substances, such as wetting or emulsifying agents, preservatives, and pH buffering agents and the like, for example, sodium acetate or sorbitan monolaurate. In particular embodiments, the carrier may be sterile.

Such compositions may be present in a sealed vial, for lyophilized for subsequent solubilization.

Recombinant: A nucleic acid molecule or polypeptide that is not naturally occurring or has a sequence that is made by an artificial combination of two otherwise separated segments of nucleotide or amino acid sequence. This artificial combination can be accomplished by chemical synthesis or by the artificial manipulation of isolated segments of nucleic acids, e.g., by genetic engineering techniques. The term “recombinant” includes nucleic acids or polypeptides that have been altered solely by addition, substitution, or deletion of a portion of a natural nucleic acid molecule or peptide.

Reporter: Reporter genes are genes whose products can be assayed (i.e., observed or detected) subsequent to their introduction into a cell or organism, for example in a mammalian cell. Reporters can be used as markers for screening successfully transfected host cells (e.g., those transfected with a non-native coronavirus genome provided herein), for studying regulation of gene expression, or can serve as controls for standardizing transfection efficiencies. Reporter gene expression can be either constitutive or inducible, with an external intervention such as, for example, the introduction of IPTG in the β-galactosidase system. Reporter genes can be expressed under their own promoter independent from that of the introduced gene or genes of interest, allowing the screening of successfully transfected cells even when the gene or genes of interest are expressed only under certain specific conditions.

For example, a reporter can include, but is not limited to, a nucleic acid, such as a transcript of a specific gene, a polypeptide product of a gene, a non-gene product polypeptide, a glycoprotein, a carbohydrate, a glycolipid, a lipid, a lipoprotein or a small molecule (for example, molecules having a molecular weight of less than 10,000 amu). A reporter gene, such as a reporter gene inserted into a coronavirus genome as disclosed herein, may encode a fluorescent molecule (such as a fluorescent protein, such as green fluorescent protein, red fluorescent protein, or yellow fluorescent protein) or a bioluminescent molecule (such as luciferase or nanoluciferase) that can be visualized. The amount of fluorescence or bioluminescence emitted from a fluorescent or bioluminescent molecule can be measured, such as the amount of fluorescence emitted from an intrinsically fluorescent molecule (for example green fluorescent protein, yellow fluorescent protein, or red fluorescent protein, among others) or a fluorophore complexed to a protein or nucleic acid. Fluorescence and bioluminescence detection methods suitable for use in the disclosed methods include conventional fluorometry, microscopy, flow cytometry, and spectroscopy. For high throughput screening, laser scanning imaging and microplate readers are also suitable.

SARS-CoV-2: Also known as 2019-nCoV or 2019 novel coronavirus, SARS-CoV-2 is a positive-sense, single stranded RNA virus of the genus betacoronavirus that has emerged as a highly fatal cause of severe acute respiratory infection, such as COVID-19. The viral genome is capped, polyadenylated, and covered with nucleocapsid proteins. The SARS-CoV-2 virion includes a viral envelope with large spike glycoproteins. The SARS-CoV-2 genome, like most coronaviruses, has a common genome organization with the replicase gene included in the 5′-two thirds of the genome, and structural genes included in the 3′-third of the genome. The SARS-CoV-2 genome encodes the canonical set of structural protein genes in the order 5′-spike (S)-envelope (E)-membrane (M) and nucleocapsid (NP)-3′. An exemplary native SARS-CoV-2 genome is provided in SEQ ID NO: 14. Symptoms of SARS-CoV-2 infection include fever and respiratory illness, such as dry cough and shortness of breath. Cases of severe infection can progress to severe pneumonia, multi-organ failure, and death. The time from exposure to onset of symptoms is approximately 2 to 14 days.

In one example, a SARS-CoV-2 is a naturally occurring variant thereof, such as alpha (B.1.1.7 and Q lineages); beta (B.1.351 and descendent lineages); delta (B.1.617.2 and AY lineages); gamma (P.1 and descendent lineages); epsilon (B.1.427 and B.1.429); eta (B.1.525); iota (B.1.526); kappa (B.1.617.1); 1.617.3; mu (B.1.621, B.1.621.1), zeta (P.2), and omicron (such as original lineage: B.1.1.529 and lineages: BA.2, BA.4, BA.5, BQ.1, BQ.1.1, BA.4.6, and BF.7). Such variants can be used to generate a non-native SARS-CoV-2 genome using the information provided herein.

SARS-CoV-2 Envelope (E): A homopentameric, 75-residue viroporin that forms a cation channel important for virus pathogenicity. The E polypeptide has a short, hydrophilic amino terminus of 7-12 amino acids, followed by a large hydrophobic transmembrane domain (TMD) of amino acids, and finally a long, hydrophilic carboxyl terminus, that comprises most of the protein. The hydrophobic region of the TMD contains at least one predicted amphipathic α-helix that oligomerizes to form an ion-conductive pore in membranes. An exemplary native RNA E sequence is provided as nt 26,245 to 26,472 of SEQ ID NO: 14.

SARS-CoV-2 Membrane (M): The M protein spans the membrane bilayer, leaving a short NH2-terminal domain outside the virus envelope and a long COOH terminus (cytoplasmic domain) inside the envelope. In silico analyses suggest that M has a triple-helix bundle and forms a single 3-transmembrane domain. An exemplary native RNA M sequence is provided as nt 26,523 to 27,191 of SEQ ID NO: 14.

SARS-CoV-2 Nucleocapsid (NP): The NP (also known as N) protein packages the positive-sense RNA genome of coronaviruses to form ribonucleoprotein structures enclosed within the viral capsid. The NP protein is the most highly expressed of the four major coronavirus structural proteins. In addition to its interactions with RNA, NP forms protein-protein interactions with the coronavirus membrane protein (M) during the process of viral assembly. NP also has additional functions in manipulating the cell cycle of the host cell. The NP protein is composed of two main domains connected by an intrinsically disordered region (IDR) (the linker region), with additional disordered segments at each terminus. A third small domain at the C-terminal tail appears to have an ordered alpha helical secondary structure and may be involved in the formation of higher-order oligomeric assemblies. An exemplary native RNA NP sequence is provided as nt 28,274 to 29,533 of SEQ ID NO: 14.

SARS-CoV-2 non-structural protein 1 (Nsp1): The Nsp1 protein suppresses host innate immune functions. On entering host cells, the SARS-CoV-2 genomic RNA is translated by the cellular protein synthesis machinery to produce a set of non-structural proteins (Nsps). Nsps render cellular conditions favorable for viral infection and viral mRNA synthesis. Nsp1 is encoded by the gene closest to the 5′ end of the viral genome and is among the first proteins to be expressed after cell entry and infection to repress multiple steps of host protein expression. SARS-CoV-2 Nsp1 binds to the human 40S subunit in ribosomal complexes, including the 43S pre-initiation complex and the non-translating 80S ribosome. The protein inserts its C-terminal domain into the mRNA channel, where it interferes with mRNA binding. An exemplary native RNA Nsp1 sequence is provided in SEQ ID NO: 60 and nt 266 to 805 of SEQ ID NO: 14.

SARS-CoV-2 non-structural protein 4 (Nsp4): The Nsp4 protein participates in the assembly of virally-induced cytoplasmic double-membrane vesicles (DMVs) necessary for viral replication. Nsp4 forms a complex with Nsp3 and Nsp6 that modifies the endoplasmic (ER) reticulum into DMVs. H120 and F121 in the lumenal loop in Nsp4 are essential for binding to Nsp3, and this interaction is crucial for viral propagation. An exemplary native RNA Nsp4 sequence is provided in SEQ ID NO: 62 and nt 8,555 to 10,054 of SEQ ID NO: 14. SARS-CoV-2 non-structural protein 10 (Nsp10): The Nsp10 protein plays a role in SARS-CoV-2 viral transcription by stimulating both Nsp14 3′-5′ exoribonuclease and Nsp16 2′-O-methyltransferase activities and therefore plays a role in viral mRNAs cap methylation. Nsp10 is translated as part of the polyprotein pp1ab, which is subsequently processed by the Main protease and Papain-like protease into individual functional proteins. Nsp10 is a single domain protein made up of 139 residues and binds two zinc ions.

Nsp10 can bind single stranded and double stranded RNA and DNA and has been shown to have an allosteric effect on Nsp14 exoribonuclease activity, which allows the exoribonuclease active site to form the substrate binding pocket, increasing activity by 35 fold. Similarly, the allosteric interaction of Nsp10 with Nsp16 allows for a more effective binding of mRNA for 2′O-methylation. An exemplary native RNA Nsp10 sequence is provided in SEQ ID NO: 64 and nt 13,025 to 13,441 of SEQ ID NO: 14.

SARS-CoV-2 Spike (S): A class I fusion glycoprotein initially synthesized as a precursor protein of approximately 1270 amino acids in size. Individual precursor S polypeptides form a homotrimer and undergo glycosylation within the Golgi apparatus as well as processing to remove the signal peptide. The S polypeptide includes S1 and S2 proteins separated by a protease cleavage site between approximately amino acid positions 685/686. Cleavage at this site generates separate S1 and S2 polypeptide chains, which remain associated as S1/S2 protomers within the homotrimer. It is believed that the beta coronaviruses are generally not cleaved prior to the low pH cleavage that occurs in the late endosome-early lysosome by the TMPRSS2 protease, at the start of the fusion peptide. Cleavage between S1/S2 is not required for function and is not observed in all viral spikes. The S1 subunit is distal to the virus membrane and contains the receptor-binding domain (RBD) that is believed to mediate virus attachment to its host receptor. The S2 subunit is believed to contain the fusion protein machinery, such as the fusion peptide, two heptad-repeat sequences (HR1 and HR2) and a central helix typical of fusion glycoproteins, a transmembrane domain, and the cytosolic tail domain. An exemplary native RNA S sequence is provided as nt 21,563 to 25,384 of SEQ ID NO: 14.

Sequence identity: The similarity between amino acid or nucleotide sequences is expressed in terms of the similarity between the sequences, otherwise referred to as sequence identity. Sequence identity is frequently measured in terms of percentage identity; the higher the percentage, the more similar the two sequences are. Homologs, orthologs, or variants of a polypeptide or polynucleotide will possess a relatively high degree of sequence identity when aligned using standard methods.

Methods of alignment of sequences for comparison are known. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. In the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

Variants of a polypeptide or nucleic acid sequence are typically characterized by possession of at least about 75%, for example, at least about 80%, 85%, 90%, 91%, 92%, 93%, 94%, 95%, 96%, 97%, 98% or 99% sequence identity counted over the full length alignment with the amino acid or nucleotide sequence of interest. Sequences with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids (or 30-60 nucleotides), and may possess sequence identities of at least 85% or at least 90% or 95% depending on their similarity to the reference sequence. Methods for determining sequence identity over such short windows are available at the NCBI website on the internet.

As used herein, reference to “at least 90% identity” (or similar language) refers to “at least 90%, at least 91%, at least 92%, at least 93%, at least 94%, at least 95%, at least 96%, at least 97%, at least 98%, at least 99%, or even 100% identity” to a specified reference sequence. Thus, a non-native SARS-CoV-2 genome having at least 90% sequence identity to SEQ ID NOs: 1, 2, 3, 4, 5, 6, 7, 8, 9, 10, 11, 12, 13, 16, or 17 is one that has at least 90%, at least 91%, at least 92%, at least 93%, at least 94%, at least 95%, at least 96%, at least 97%, at least 98%, at least 99%, or even 100% identity to SEQ ID NO: 1, 2, 3, 4, 5, 6, 7, 8, 9, 10, 11, 12, 13, 16, or 17, respectively.

Stable cell clone: A host cell that has integrated an exogenous nucleic acid molecule into its genome, replicates the exogenous nucleic acid molecule. Stable cell clones can in some examples indefinitely reproduce, and express the exogenous nucleic acid molecule. In some examples, stable cell clones are genetically homogeneous. In some examples, growth in the presence of a selectable marker, such as an antibiotic, ensures that only cells with the exogenous nucleic acid molecule continue to be viable.

For example, a stable host cell clone that includes non-native coronavirus replicon provided herein (such as any one of SEQ ID NOs: 1, 2, 3, 4, 5, 6, 7, 8, 9, 10, 11, 12, or 13), is one that includes the coronavirus replicon in its genome, and autonomously replicates the non-native coronavirus replicon. In some examples, descendants of a stable cell clone are genetically identical, for example they express the same non-native coronavirus replicon, and in some examples include the same number of non-native coronavirus replicons. In some examples, a stable cell clone can be grown for at least 10 generations, at least 50 generations, at least 100 generations, or at least 1000 generations, such as 10-10,000 generations, 10-1000 generations, 10-500 generations, 10-100 generations, 10-50 generations, 10-20 generations, 100-5000 generations, or 100-500 generations, and retain genetic homogeneity.

Transfection and Transduction: A transfected cell is a cell into which has been introduced a nucleic acid molecule by molecular biology techniques. Transfection encompasses all techniques by which a nucleic acid molecule might be introduced into such a cell, including transfection with plasmid vectors, and introduction of DNA by electroporation, liposome-mediated transfection (lipofection), non-liposomal transfection, dendrimer-based transfection, particle bombardment, and microinjection Transduction as used herein includes virus-mediated gene delivery.

### Overview

The development of antivirals against severe acute respiratory syndrome coronavirus 2 (SARS-CoV-2) has been hampered by the lack of efficient cell-based systems that are amenable to high-throughput screens in biosafety level 2 (BSL2) laboratories. The present disclosure provides stable cell clones harboring autonomously replicating SARS-CoV-2 RNAs without functional S, M, and E genes, efficiently derived from the baby hamster kidney (BHK-21) cell line when a pair of mutations were introduced into the non-structural protein 1 (Nsp1) of SARS-CoV-2 to ameliorate cellular toxicity associated with virus replication. These stable cell clones, which harbor autonomously replicating SARS-CoV-2 RNA without producing infectious virus, can be readily cultured in most industrial laboratory settings, including BSL-2 laboratory conditions, for high-throughput drug screen. A 272-compound library was screened in stable cell clones and three compounds were identified as novel inhibitors of SARS-CoV-2 replication. Thus, provided herein is a robust, cell-based system for genetic and functional analyses of SARS-CoV-2 replication and for the development of antiviral drugs, such as those that can reduce or inhibit SARS-CoV-2 replication, for example by at least 10%, at least 20%, at least 30%, at least 40%, at least 50%, at least 60%, at least 70%, at least 80%, at least 90%, at least 99%, at least 99%, or 100%, for example as compared to an amount of SARS-CoV-2 replication without treatment with the drug.

The data herein show that stable cell clones harboring a mutated SARS-CoV-2 replicon may be derived when K164A/H165A mutations are introduced to the SARS-CoV-2 Nsp1 gene. The ^{K164A/H165A} mutations reduced the interaction between the C-terminus of Nsp1 and a ribosome and hence increased the accessibility of ribosomes to host mRNA.

By contrast, R124S/K125E mutations reduced the binding of Nsp1 N-terminus to the 5′-UTR of viral mRNA, leaving the C-terminal of Nsp1 constantly bound to ribosome. Consequently, neither viral nor host mRNA could efficiently access the ribosome in the presence of Nsp1 R124S/K125E mutations. However, N128S/K129E mutations, failed to render viable cells in the replicon systems described herein. Additionally, viable cells were only recovered from the BHK-21 cell line, indicating either that alleviation of Nsp1-mediated cytotoxicity by K164A/H165A is restricted to the BHK-21 cell line or that there are additional viral factors that cause cell death in other cell types.

### Mutated/Non-Native SARS-Cov-2 Genomes

Provided herein are isolated, non-native coronavirus genomes, compositions comprising the coronavirus genomes, isolated host cells comprising the coronavirus genomes, and methods of using the cells that comprise the coronavirus genomes, such as methods of using the cells to identify anti-viral compounds (such as those that can treat SARS-CoV-2 infection). As demonstrated in the Examples, the disclosed coronavirus genomes are replication-competent (i.e., the coronavirus genomes replicate autonomously in cells harboring the coronavirus genomes), but do not produce infectious virus. Thus, cells harboring the disclosed coronavirus genomes may be cultured in, for example, a standard BSL-2 laboratory, such as for high throughput screening of anti-viral compound libraries.

In some embodiments, an isolated, non-native coronavirus genome disclosed herein comprises genetically inactivated coronavirus S, E, and M genes. In some embodiments, the isolated, non-native coronavirus genome comprises insertion of a coding sequence for a marker gene, and/or insertion of a coding sequence for a reporter gene. In a non-limiting example, a coding sequence for a marker gene can replace a coding sequence for native coronavirus E and M genes. In another non-limiting example, a coding sequence for a reporter gene can replace a coding sequence for a native coronavirus S gene. Inactivated S, E, and M genes may include one or more inactivating nucleotide mutations, insertions, and/or deletions. A marker gene may be a selectable marker gene, such as an antibiotic resistance gene, for example an antibiotic resistance gene conferring resistance to neomycin (such as a NeoR gene encoding a neomycin phosphotransferase enzyme), kanamycin, geneticin, ampicillin, or a combination thereof. A reporter gene may encode a fluorescent or bioluminescent molecule, such as, for example, a nanoluciferase enzyme.

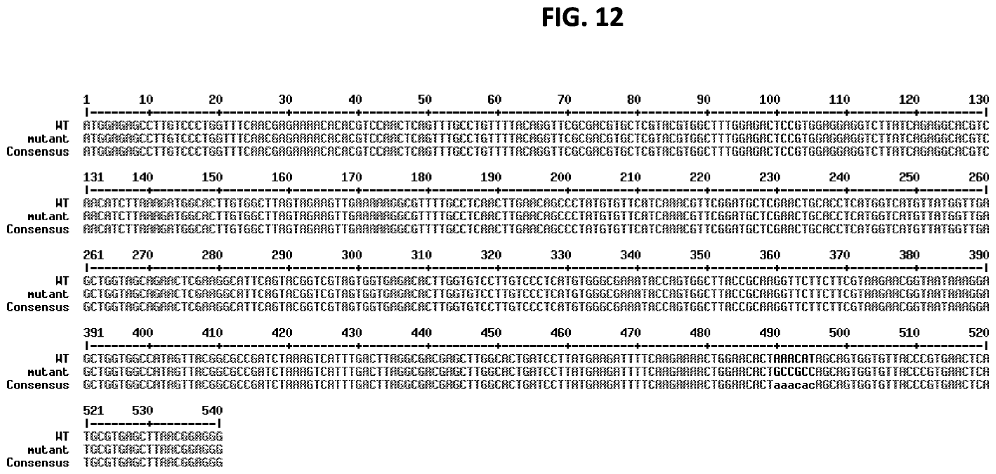

In further embodiments, the isolated, non-native coronavirus genome includes mutations in a coronavirus Nsp1 gene (e.g., mutations relative to the exemplary native Nsp1 nucleic acid sequence of SEQ ID NO: 60). Such mutations can alleviate Nsp1-mediated cytotoxicity. In some embodiments, the mutations in a coronavirus Nsp1 gene that reduce cellular toxicity are K164A and H165A mutations. The Nsp1 gene K164A substitution can be encoded by guanine, cytosine, and cytosine (GCC) residues at Nsp1 nucleotides 490, 491, and 492, respectively, and the H165A substitution can be encoded by guanine, cytosine, and cytosine (GCC) residues at Nsp1 nucleotides 493, 494, and 495, respectively. SEQ ID NO: 59 is an example Nsp1 nucleotide sequence where both K164A and H165A mutations are present. In some examples, the numbering of the Nsp1 nucleotides (or corresponding encoded amino acids) is based on SEQ ID NO: 59 or FIG. 12.

In other embodiments, an isolated, non-native coronavirus genome disclosed herein includes mutations, such as a R401S mutation, in a coronavirus Nsp4 gene (e.g., mutations relative to the exemplary native Nsp4 nucleic acid sequence of SEQ ID NO: 62, such as shown in SEQ ID NO: 61). In yet other embodiments, the isolated, non-native coronavirus genome includes mutations, such as a T111I mutation, in a coronavirus Nsp10 gene (e.g., mutations relative to the exemplary native Nsp10 nucleic acid sequence of SEQ ID NO: 64, such as shown in SEQ ID NO: 63). In some embodiments, the isolated, non-native coronavirus genome further comprises a genetically inactivated NP gene. The inactivated NP gene may include one or more inactivating nucleotide mutations, insertions, and/or deletions. In some examples, the numbering of the Nsp4 nucleotides (or corresponding encoded amino acids) is based on SEQ ID NO: 61 or 62. In some examples, the numbering of the Nsp10 nucleotides (or corresponding encoded amino acids) is based on SEQ ID NO: 63 or 64.

In certain embodiments, the isolated, non-native coronavirus genome is a non-native betacoronavirus genome, such as a SARS-CoV genome, a SARS-CoV-2 genome, or a MERS-CoV genome. In such embodiments, the isolated, non-native coronavirus genome is at least 20,000 kb in length, such as at least 21,000 kb, at least 22,000 kb, at least 23,000 kb, at least 24,000 kb, at least 25,000, at least 26,000, at least 27,000, at least 28,000, at least 29,000 or at least 30,000 kb in length. In some embodiments, the isolated, non-native coronavirus genome is at least 24,000-27,000 kb in length. In specific, non-limiting embodiments, the nucleotide sequence of an isolated, non-native SARS-CoV-2 genome disclosed herein is at least 80%, at least 85%, at least 90%, at least 95%, at least 96%, at least 97%, at least 98%, at least 99%, at least 99.9% identical, or 100% identical to a nucleotide sequence set forth as SEQ ID NO: 1, 2, 3, 4, 5, 6, 7, 8, 9, 10, 11, 12, or 13. In other specific, non-limiting embodiments, the nucleotide sequence of the isolated, non-native SARS-CoV-2 genome comprises or consists of a nucleotide sequence set forth as SEQ ID NO: 1, 2, 3, 4, 5, 6, 7, 8, 9, 10, 11, 12, or13.

In certain embodiments, the isolated, non-native coronavirus genome is a DNA molecule. In certain embodiments, the isolated, non-native coronavirus genome is an RNA molecule.

### Methods of Genetic Inactivation of a SARS-CoV-2 Spike, Envelope, and Membrane Gene, and Optionally a Nucleocapsid Gene

As used herein, an “inactivated” or “functionally deleted” coronavirus S, E, M, or NP gene (such as a SARS-CoV-2 S, E, M, or NP gene) means that the gene has been mutated, for example by insertion, deletion, or substitution (or combinations thereof) of one or more nucleotides such that the mutation substantially reduces (and in some cases abolishes) expression or biological activity of the encoded gene product. For example, a genetically inactivated S gene/protein can have a reduction in expression/activity of at least 50%, at least 75%, at least 90%, at least 95%, at least 99%, or 100% (complete elimination of expression/activity), as compared to S gene/protein expression/activity of a native S sequence. For example, a genetically inactivated E gene/protein can have a reduction in expression/activity of at least 50%, at least 75%, at least 90%, at least 95%, at least 99%, or 100% (complete elimination of expression/activity), as compared to E gene/protein expression/activity of a native E sequence. For example, a genetically inactivated M gene/protein can have a reduction in expression/activity of at least 50%, at least 75%, at least 90%, at least 95%, at least 99%, or 100% (complete elimination of expression/activity), as compared to M gene/protein expression/activity of a native M sequence. For example, a genetically inactivated NP gene/protein can have a reduction in expression/activity of at least 50%, at least 75%, at least 90%, at least 95%, at least 99%, or 100% (complete elimination of expression/activity), as compared to NP gene/protein expression/activity of a native NP sequence. The mutation can act through affecting transcription or translation of the coronavirus S, E, M, or NP gene or the mRNA of the coronavirus S, E, M, or NP gene, or the mutation can affect the coronavirus S, E, M, or NP polypeptide product itself (such as a SARS-CoV-2 S, E, M, or NP polypeptide) in such a way as to render it substantially inactive.

In one example, a cell, such as a mammalian cell, such as a baby hamster kidney cell (such as a BHK-21 cell) is transfected with a heterologous nucleotide, such as an isolated, non-native coronavirus genome (such as a SARS-CoV-2 genome disclosed herein), which has the effect of down-regulating or otherwise inactivating expression and activity a coronavirus S, E, and M (and optionally also NP) gene in the resulting non-infectious virus. This can be done by mutating control elements such as promoters and the like which control gene expression, by mutating the coding region of the gene so that any protein expressed is substantially inactive, or by deleting the coronavirus S, E, M, or NP gene entirely. For example, a coronavirus S, E, M, or NP gene can be functionally deleted by complete or partial deletion mutation (for example by deleting a portion of the coding region of the gene) or by insertional mutation (for example by inserting a sequence of nucleotides into the coding region of the gene, such as a sequence of about 1-5000 nucleotides). In one example, the coronavirus S, E, M, or NP gene is genetically inactivated by inserting coding sequences for at least one exogenous nucleic acid molecule which genetically inactivates an endogenous coronavirus S, E, M, or NP gene. In one example, the isolated, non-native coronavirus genome having genetically inactivated coronavirus S, E, and M (and in some examples also NP) genes (such as a SARS-CoV-2 genome disclosed herein, such as a SARS-CoV-2 genome having a nucleotide sequence at least 80%, at least 85%, at least 90%, at least 95%, at least 96%, at least 97%, at least 98% or at least 99% identical to a nucleotide sequence set forth as SEQ ID NO: 1, 2, 3, 4, 5, 6, 7, 8, 9, 10, 11, 12, or 13) replicates autonomously in a cell harboring the coronavirus genome. In some examples, the isolated, non-native coronavirus genome does not produce infectious viruses.

In particular examples, an insertional mutation includes introduction of a sequence that is in multiples of three bases (e.g., a sequence of 3, 9, 12, or 15 nucleotides) to reduce the possibility that the insertion will be polar on downstream genes. For example, insertion or deletion of even a single nucleotide that causes a frame shift in the open reading frame, which in turn can cause premature termination of the encoded coronavirus S, E, and M (and in some examples also NP) polypeptide or expression of a substantially inactive polypeptide. Mutations can also be generated through insertion of foreign gene sequences, for example the insertion of a gene encoding antibiotic resistance (such as neomycin, kanamycin, geneticin, and/or ampicillin).

In one example, genetic inactivation is achieved by deletion of a portion of the coding region of an endogenous coronavirus S, E, M, and/or NP gene. For example, some, most (such as at least 50%) or virtually the entire endogenous coding region can be deleted. In particular examples, about 5% to about 100% of the endogenous gene is deleted, such as at least 20% of the gene, at least 40% of the gene, at least 75% of the gene, at least 90%, or 100% of the endogenous coronavirus S, E, M, and/or NP gene. In specific, non-limiting examples, about 5% to about 100% of the endogenous S gene, such as at least 20% of the S gene, at least 40% of the S gene, at least 75% of the S gene, at least 90%, or substantially 100% of the S gene is replaced by a reporter gene (such as a NanoLuc gene) in an isolated, non-native coronavirus genome. In other specific, non-limiting examples, about 5% to about 100% of the endogenous E and M genes, such as at least 20% of the E and M genes, at least 40% of the E and M genes, at least 75% of the E and M genes, at least 90%, or 100% of the E and M genes is replaced by a marker gene (such as a selectable marker gene, such as an antibiotic resistance gene, such as a NeoR gene) in an isolated, non-native coronavirus genome.

Deletion mutants can be constructed using any technique. Thus, for example, an isolated, non-native coronavirus genome can be engineered to have disrupted coronavirus S, E, and M (and in some examples also NP) genes using mutagenesis technology.

In some embodiments, expression of one or more coronavirus genes, such as expression of SARS-CoV-2 S, E, and M (and in some examples also NP) gene, is inhibited at least about 10%, at least about 25%, at least 50%, at least 75%, at least 80%, at least 85%, at least 90%, at least 95%, or 100% relative to a control, such as a native SARS-CoV-2. In a specific, non-limiting example, expression of a SARS-CoV-2 S gene is inhibited at least about 10%, at least about 25%, at least 50%, at least 75%, at least 80%, at least 85%, at least 90%, at least 95%, or 100% relative to a control, such as a native SARS-CoV-2.

Furthermore, sequences for coronavirus genes, such as coronavirus (such as SARS-CoV-2) S, E, M, and NP genes, are publicly available. The specific sequences listed herein are provided for reference only and are not intended to be limiting.

Various delivery systems can be used to introduce a non-native coronavirus genome/replicon into a cell, such as a mammalian cell. Such systems include, for example, encapsulation in liposomes, microparticles, microcapsules, and nanoparticles.

Measuring SARS-CoV-2 S, E, M, and/or NP Gene Inactivation

An isolated, non-native coronavirus genome having inactivated endogenous coronavirus S, E, and M (and in some examples also NP) gene (such as a SARS-CoV-2 genome having inactivated endogenous coronavirus S, E, and M (and in some examples also NP) genes as disclosed herein) can be identified. For example, PCR and nucleic acid hybridization techniques, such as Northern and Southern analysis, can be used to confirm that a coronavirus genome has a genetically inactivated coronavirus S, E, and M (and in some examples also NP) gene. Similarly, next generation sequencing techniques can be used to confirm that a coronavirus genome has a genetically inactivated coronavirus S, E, and M (and in some examples also NP) gene. In one example, quantitative reverse transcription PCR (qRT-PCR) is used for detection and quantification of targeted messenger RNA, such as mRNA of a coronavirus S, E, and M (and in some examples also NP) gene in the parent and mutant strains, such as viral RNA produced in a cell (such as a BHK-21 cell) harboring the isolated, non-native coronavirus genome.

Immunohistochemical and biochemical techniques can also be used to determine if a cell harboring an isolated, non-native coronavirus genome expresses coronavirus S, E, and M (and in some examples also NP) by detecting the expression of coronavirus S, E, M, and/or NP peptides encoded by coronavirus S, E, M, and/or NP genes, respectively. For example, an antibody having specificity for coronavirus S, E, M, or NP can be used to determine whether or not a particular coronavirus genome contains a functional nucleic acid encoding a coronavirus S, E, M, and/or NP protein. Further, biochemical techniques can be used to determine if a cell contains a coronavirus S, E, M, and/or NP gene inactivation by detecting a product produced as a result of the lack of expression of the peptide.

### Mutations in SARS-CoV-2 Non-Structural Proteins

In some embodiments, an isolated, non-native coronavirus genome disclosed herein includes one or more mutations in one or more coronavirus genes, such as mutations in a non-structural protein (Nsp) gene, that reduce or removes toxicity (such as toxicity resulting from expression of the mutated one or more genes) to a cell into which the isolated, non-native coronavirus genome has been introduced, such as by transfection. In some embodiments, the isolated, non-native coronavirus genome comprises a coding sequence for a coronavirus Nsp1 protein comprising one or more (such as two, for example two consecutive) amino acid substitutions to reduce or remove Nsp1-mediated toxicity to a cell into which the isolated, non-native coronavirus genome has been introduced. Such amino acid substitutions may reduce cellular toxicity by weakening the interaction between the Nsp1 C-terminus and a ribosome, potentially leading to a shorter occupation time of Nsp1 on the ribosome and enhancing accessibility of the ribosome to host mRNA. In some embodiments, the Nsp1-mediated toxicity is reduced or removed by K164A and H165A substitutions. In some embodiments, the Nsp1 gene K164A substitution is encoded by guanine, cytosine, and cytosine (GCC) residues, such as at nucleotides 755, 756, and 757, respectively, of SEQ ID NO: 1 or nucleotides 490, 491, and 492, respectively of SEQ ID NO: 59. In some embodiments, the Nsp1 gene H165A substitution is encoded by guanine, cytosine, and cytosine (GCC) residues, such as at nucleotides 758, 759, and 760, respectively, of SEQ ID NO: 1 or nucleotides 493, 494, and 495, respectively, of SEQ ID NO: 59.

In some embodiments, the isolated, non-native coronavirus genome further includes a non-structural protein 4 (Nsp4) gene encoding a R401S substitution (e.g., as shown in SEQ ID NO: 61), a non-structural protein 10 (Nsp10) gene encoding a Ti111I substitution (e.g., as shown in SEQ ID NO: 63), or both substitutions. In some embodiments, the Nsp4 R401S substitution is encoded by adenosine, guanine, and thymine residues (AGT), such as at nucleotides 1,201, 1,202, and 1,203, respectively, of SEQ ID NO: 61 (9,755, 9,756, and 9,757, respectively, of clone 2, SEQ ID NO: 2). In some examples, the numbering of the Nsp4 nucleotides (or corresponding encoded amino acids) is based on SEQ ID NO: 61. In other embodiments, the Nsp10 Ti111I substitution is encoded by adenine, thymine, and adenine residues (ATA), such as at nucleotides 331, 332, and 333, respectively, of SEQ ID NO: 63 (13,355, 13,356, and 13,357, respectively, of clone 3, SEQ ID NO: 3). In some examples, the numbering of the Nsp10 nucleotides (or corresponding encoded amino acids) is based on SEQ ID NO: 63.

### Exemplary Markers

In some embodiments of the disclosed isolated, non-native coronavirus genome, the genome includes an insertion of at least one marker gene, such as a selectable marker. In specific, non-limiting embodiments, the coronavirus E and M genes of the isolated, non-native coronavirus genome are replaced with a selectable marker gene. In specific, non-limiting embodiments, the coronavirus S gene of the isolated, non-native coronavirus genome is replaced with a selectable marker gene. A selectable marker can include an antibiotic resistance gene, such as an antibiotic resistance gene that confers resistance to neomycin, G418 (an analog of neomycin), zeocin, blasticidin, puromycin, kanamycin, geneticin, ampicillin, or another antibiotic, or a combination of antibiotics.

In specific, non-limiting embodiments, the coronavirus E and M genes (or the S gene) of the isolated, non-native coronavirus genome are replaced with one or more marker genes (such as 1, 2, or 3 of such genes), such as a selectable marker, such as a NeoR gene. The NeoR gene encodes the neomycin phosphotransferase enzyme and confers resistance to neomycin and its analogs (such as the antibiotic G418) in cells expressing a nucleic acid molecule encoding NeoR, such as in cells transfected with an isolated, non-native coronavirus genome encoding NeoR as disclosed herein.

In particular, non-limiting embodiments, the NeoR gene comprises at least 80%, at least 85%, at least 90%, at least 92%, at least 95%, at least 96%, at least 97%, at least 98%, at least 99%, or 100% sequence identity to nucleotides 22,939 to 23,733 of SEQ ID NO: 1.

### Exemplary Reporters

Reporter genes and detection systems can be used herein to determine whether an isolated, non-native coronavirus genome has been successfully introduced into a cell, for example whether a cell has been successfully transfected with an isolated, non-native SARS-CoV-2 coronavirus genome as described herein. In specific, non-limiting embodiments, the coronavirus E and M genes of the isolated, non-native coronavirus genome are replaced with one or more reporter genes, such as 1, 2, or 3 reporter genes. In specific, non-limiting embodiments, the coronavirus S gene of the isolated, non-native coronavirus genome is replaced with a reporter gene. A reporter gene as disclosed herein, such as a reporter gene inserted into an isolated, non-native coronavirus genome, can encode a fluorophore, such as green fluorescent protein (GFP), or a bioluminescent molecule, such as luciferase (such as Firefly or Renilla luciferase) or nanoluciferase, that can be visualized (e.g., using microscopy, flow cytometry, spectroscopy). Such reporters and detection systems are used in mammalian cell culture systems. Such detection can be qualitative or quantitative.

The amount of fluorescence emitted from a fluorescent molecule (for example green fluorescent protein, red fluorescent protein, yellow fluorescent protein, and others) can be measured, such as the amount of fluorescence emitted from an intrinsically fluorescent molecule or a fluorophore complexed to a protein or nucleic acid. Fluorescence detection methods suitable for use in the disclosed methods include conventional fluorometry, fluorescence microscopy, flow cytometry, and fluorescence spectroscopy. For high throughput screening, laser scanning imaging and microplate fluorescence readers are also suitable.

In contrast to fluorescent reporters, bioluminescent reporters generate de novo light without the need for external excitation through photons, and they are highly sensitive with a broad dynamic range. The luciferin reporter bioluminescent signal is generated through oxidation of a substrate (luciferin) by the luciferase enzyme and there are many luciferin/luciferase pairs. The luciferase enzyme catalyzes a reaction with its substrate (e.g., luciferin) to produce yellow-green or blue light, depending on the luciferase gene. Since light excitation is not needed for luciferase bioluminescence, there is minimal autofluorescence and thus virtually background-free fluorescence.

Nanoluciferase (NLuc) is a small (19.1 kDa) luciferase enzyme that catalyzes the conversion of its substrate, furimazine, to furimamide to produce high intensity, glow-type luminescence. NLuc does not require post-translational modifications in mammalian cells, unlike green fluorescent protein, and allows for assaying of live cells, such as using luminescence microscopy.

In some embodiments of the disclosed isolated, non-native coronavirus genomes, the coronavirus genome includes a reporter gene. In some embodiments, the coronavirus S gene in the isolated, non-native coronavirus genome is replaced with a reporter gene, such as a reporter gene encoding a fluorescent or bioluminescent reporter molecule, such as a reporter gene encoding nanoluciferase (such as NanoLuc).

In particular, non-limiting embodiments, the NanoLuc gene comprises at least 80%, at least 85%, at least 90%, at least 92%, at least 95%, at least 96%, at least 97%, at least 98%, at least 99%, or 100% sequence identity to nucleotides 21,563 to 22,078 of SEQ ID NO: 1.

### Cells Including Mutated/Non-Native SARS-Cov-2 Genomes

Disclosed herein are cells, such as mammalian cells, into which an isolated, non-native coronavirus genome (such as a DNA or RNA molecule) has been introduced, such as through transfection. In some embodiments, the host cell is a mammalian host cell, such as a human host cell. Techniques for the propagation of mammalian cells in culture are known (see, e.g., Helgason and Miller (Eds.), 2012, Basic Cell Culture Protocols (Methods in Molecular Biology), 4th Ed., Humana Press). Examples of mammalian host cell lines that can be used include Vero cells, HeLa cells, CHO cells, WI38 cells, BHK cells, HEK293 cells or derivatives thereof and COS cell lines, although cell lines may be used, such as cells designed to provide higher expression, desirable glycosylation patterns, or other features. In some embodiments, the host cells include HEK293 cells or derivatives thereof, such as GnTI−/− cells (ATCC® No. CRL-3022), or HEK-293F cells.

In some embodiments, cells of the present disclosure are mammalian cells, such as baby hamster kidney cells. In a specific, non-limiting embodiment, the cells are BHK-21 cells. In another specific, non-limiting embodiment, the cells are BHK-21 cells (BHK-21-NP^{Dox-ON}) in which a coronavirus nucleocapsid protein (such as a SARS-CoV-2 nucleocapsid protein) is stably expressed in a doxycycline-inducible manner. In another specific, non-limiting embodiment, the cells are the cells deposited as ATCC #______.

A transfected cell is a cell into which (or into an ancestor of which) has been introduced, such as by means of recombinant nucleic acid molecule techniques, a nucleic acid molecule encoding an isolated, non-native coronavirus genome disclosed herein. Transfection of a host cell with a disclosed non-native coronavirus genome, may be carried out by methods including calcium phosphate coprecipitation, microinjection, electroporation, liposome-mediated transfection, non-liposomal transfection, dendrimer-based transfection, particle bombardment, microinjection, and others. Eukaryotic cells can also be co-transfected with DNA sequences encoding an isolated, non-native coronavirus genome (e.g., encoding an RNA genome) and a second nucleic acid molecule, such as a nucleic acid encoding a coronavirus nucleocapsid protein.

In some examples, the identification and characterization of a successfully transfected cell is by expression of a certain marker or different expression levels and patterns of more than one marker. That is, the presence or absence, the high or low expression, of one or more marker(s) typifies and identifies a successfully transfected cell. The expression of certain markers can be determined by measuring the level at which the marker is present in the cells of the cell culture or cell population, or in the supernatant of the cell culture or cell population, as compared to a standardized or normalized control marker. In such processes, the measurement of marker expression can be qualitative or quantitative. One method of quantitating the expression of markers that are produced by marker genes is use of quantitative PCR (Q-PCR). In some embodiments, the presence, absence and/or level of expression of a marker is determined by quantitative PCR (Q-PCR).

In other embodiments, immunohistochemistry is used to detect the proteins expressed by a gene or genes of interest. In still other embodiments, Q-PCR can be used in conjunction with immunohistochemical techniques or flow cytometry techniques to effectively and accurately characterize and identify cell types and determine both the amount and relative proportions of such markers in a subject cell type. In one embodiment, Q-PCR can quantify levels of expression in a cell culture containing a population of cells. In another embodiment, Q-PCR is used in conjunction with flow cytometry methods to characterize and identify transfected cells. Thus, by using a combination of the methods described herein, and such as those described above, complete characterization and identification of cells into which an isolated, non-native coronavirus genome has been introduced can be accomplished and demonstrated. For example, in one embodiment, cells (such as BHK-21 cells) transfected with an isolated, non-native coronavirus genome express at least a reporter gene (such as Nanoluc), a marker gene (such as NeoR), and coronavirus N, ORF3a, ORF7a, ORF8, and ORF10, but do not express coronavirus S, E, or M genes. In some embodiments, such cells do not express the coronavirus NP gene.

Still other methods can also be used to quantitate marker gene expression. For example, the expression of a marker gene product can be detected by using antibodies specific for the marker gene product of interest (e.g., Western blot, ELISA, flow cytometry analysis, and the like). In certain processes, the expression of marker genes characteristic of cells into which an isolated, non-native coronavirus genome has been introduced as well as the lack of significant expression of marker genes characteristic of such cells.

Reporter genes and detection systems can also be used herein to determine whether an isolated, non-native coronavirus genome has been successfully introduced into a cell, for example whether a cell has been successfully transfected with an isolated, non-native SARS-CoV-2 coronavirus genome as described herein. A reporter gene as disclosed herein, such as a reporter gene inserted into an isolated, non-native coronavirus genome, can encode a fluorophore, such as green fluorescent protein (GFP), or a bioluminescent molecule, such as luciferase (such as Firefly or Renilla luciferase) or nanoluciferase, that can be visualized.

The amount of fluorescence emitted from a fluorescent molecule (for example green fluorescent protein, red fluorescent protein, yellow fluorescent protein, and others) can be measured, such as the amount of fluorescence emitted from an intrinsically fluorescent molecule or a fluorophore complexed to a protein or nucleic acid. Fluorescence detection methods suitable for use in the disclosed methods include conventional fluorometry, fluorescence microscopy, flow cytometry, and fluorescence spectroscopy. For high throughput screening, laser scanning imaging and microplate fluorescence readers are also suitable.

In contrast to fluorescent reporters, bioluminescent reporters generate de novo light without the need for external excitation through photons, and they are highly sensitive with a broad dynamic range. The luciferin reporter bioluminescent signal is generated through oxidation of a substrate (luciferin) by the luciferase enzyme and there are many luciferin/luciferase pairs. The luciferase enzyme catalyzes a reaction with its substrate (e.g., luciferin) to produce yellow-green or blue light, depending on the luciferase gene. Since light excitation is not needed for luciferase bioluminescence, there is minimal autofluorescence and thus virtually background-free fluorescence.

In some embodiments of the disclosed cells containing an isolated, non-native coronavirus genome, the coronavirus genome includes a reporter gene. In some embodiments, the coronavirus S gene in the isolated, non-native coronavirus genome is replaced by a reporter gene, such as a reporter gene encoding a bioluminescent reporter molecule, such as a reporter gene encoding nanoluciferase (NanoLuc). Cells transfected or transduced with the isolated, non-native coronavirus genome comprising the reporter gene (such as an isolated, non-native coronavirus genome in which the coronavirus S gene has been replaced with NanoLuc) can be identified through detection of a reaction catalyzed by the expression product of the reporter gene. For example, luminescence microscopy can be used herein to detect light generated by the conversion of furimazine to furimamide by the NanoLuc reporter gene product, nanoluciferase.

### Screening Methods

Disclosed herein are methods of identifying anti-viral compounds using host cells that stably express an isolated, non-native coronavirus genome. In some embodiments, the methods are used to identify compounds that reduce or inhibit SARS-CoV-2 replication, for example by at least 10%, at least 20%, at least 30%, at least 40%, at least 50%, at least 60%, at least 70%, at least 80%, at least 90%, at least 99%, at least 99%, or 100%, for example as compared to an amount of SARS-CoV-2 replication and/or infection without treatment with the compound. In some embodiments, the method includes contacting the host cell with one or more compounds, determining a level of expression of a reporter gene in the contacted cells, and comparing the level of expression of the reporter gene in the contacted cells to a control. In some embodiments, reduced expression of the reporter gene in the contacted cells relative to the control (for example an untreated cell) indicates the compound is an anti-viral compound. In some examples, the method includes determining an IC₅₀ value for the one or more compounds.

In some examples, the method is a high-throughput screening method. Using automation, data processing/control software, liquid handling devices, and sensitive detectors, high-throughput screening allows for rapid testing of high numbers (such as thousands or millions) of molecules to determine the efficacy of each molecule for a desired purpose, such as the efficacy of each molecule for use as an anti-viral therapeutic. In some examples, identification of anti-viral compounds using the cells is carried out in microplates, such as 24-well, 48-well, 96-well or 384-well microtiter plates. In some embodiments, the fluorescence or bioluminescence intensity is detected using a microplate reader. A microplate reader detects biological, chemical, or physical events in microtiter plates. A high-intensity lamp passes light to the microtiter well and the light emitted by the reaction in the well is quantified by a detector. Detection modes for microplate assays include absorbance, fluorescence intensity, luminescence, time-resolved fluorescence, and fluorescence polarization. In some embodiments, fluorescence intensity is measured using a microplate reader, such as SPECTRAMAX® M5 (Molecular Devices), ELX800™ Absorbance Microplate Reader (BioTek), SpectraFluor (Tecan), or VICTOR3™ (Perkin Elmer). In some embodiments, the wavelength of light used for excitation is from about 485 nm to about 510 nm, such as about 485 nm to about 505 nm, such as about 495 nm to about 500 nm. The emitted light is detected at about 520 nm to about 560 nm, such as about 530 nm to about 550 nm, such as about 535 nm to about 540 nm. In a particular example, fluorescence intensity is measured using a Tecan SpectraFluor microplate reader using excitation at 485 nm and measuring emission at 535 nm.

In some embodiments, the method includes contacting cells with one or more test compounds, such as adding one or more test compounds to intact host cells stably expressing an isolated, non-native coronavirus genome as disclosed herein. The test sample is incubated with the intact cells for an amount of time to permit the molecules to enter the cells. The test compound may be incubated with the cells from about 1 to about 120 minutes, such as from about 10 minutes to about 100 minutes, about 20 minutes to about 90 minutes, about 30 minutes to about 80 minutes, about 40 minutes to about 70 minutes, about 50 minutes to about 60 minutes, such as at least about five minutes, for example about 5 minutes, 10 minutes, 15 minutes, 20 minutes, 30 minutes, 60 minutes, 90 minutes, or 120 minutes. The incubation is carried out at a temperature which permits the test compound to cross the cell membrane. For example, the incubation of the test compound with the intact cells may be at about room temperature, such as at a temperature of about 20° C. to about 25° C. In additional embodiments, the cells are incubated at a temperature of about 4° C. to about 56° C., such as about 15° C. to about 50° C., about 22° C. to about 45° C., about 25° C. to about 40° C., or about 30° C. to about 37° C. In a particular example, the test compound is incubated with intact cells for 20 minutes at room temperature. A negative control can include cells incubated under the same conditions, but without the test compound. A positive control can include cells incubated under the same conditions, but with a known anti-viral compound.

In some embodiments, the host cells are washed following incubation with the test compound to remove any test compound material that has not crossed the cell membrane. Washing may be by standard methods, for example by centrifugation of the cells, removal of the resulting supernatant, and resuspension of the cells in a solution. The cells may be resuspended in a physiological buffer, such as phosphate-buffered saline, Hank's balanced salt solution, lactated Ringer's solution, or cell culture media (for example RPMI-1640). The buffers may contain small amounts of solvent (such as about 0.5% to about 2% ethanol or methanol) or carrier molecules (such as about 1% to about 4% glucose or fructose). The wash step may be repeated one to six times, such as one time, two times, three times, four times, five times, or six times. In a particular example, the cells are washed three times by centrifugation at about 400×g for about 2 to about 10 minutes, removal of the resulting supernatant, and resuspension in phosphate buffered saline.

In some embodiments of the disclosed methods, the method is performed in a biological safety (“biosafety”) level 2 (BSL-2) laboratory. A biological safety level (BSL-1, -2, -3, or -4) is assigned to a biological lab as a safeguard to protect laboratory personnel, as well as the surrounding environment and community. The United States Centers for Disease Control (CDC) recommends that virus isolation and characterization of viral agents from SAR-CoV-2 specimens must be processed within a BSL-3 laboratory space using BSL-3 procedures. This includes any culture involving cells isolated from, or exposed, to SARS-CoV or SARS-CoV-2 patient tissues that may be permissive to virus replication. Because of the enhanced security and safety requirements associated with SARS-CoV and SARS-CoV-2 viruses (and related coronaviruses), researchers are limited in the ability to assess anti-viral compounds for treatment of human and other animal subjects infected with the viruses.

BSL-2 level covers laboratories that work with agents associated with human diseases (i.e., pathogenic or infections organisms) that pose a moderate health hazard. Examples of agents typically worked with in a BSL-2 include equine encephalitis viruses and HIV, as well as Staphylococcus aureus (staph infections). BSL-2 laboratories maintain the same standard microbial practices as BSL-1 labs, but also include enhanced measures due to the potential risks associated with the aforementioned microbes. Thus, the disclosed cells stably expressing the disclosed non-native coronavirus genome, but do not produce infectious virus, can be used in a BSL-2 laboratory, instead of a BLS-3 laboratory.

Access to a BSL-2 lab is far more restrictive than a BSL-1 laboratory. Outside personnel, or those with an increased risk of contamination, are often restricted from entering when work is being conducted. In addition to BSL-1 laboratory expectations, the following practice exemplify additional practices required in a BSL 2 lab setting: Appropriate personal protective equipment (PPE) must be worn, including lab coats and gloves. Eye protection and face shields can also be worn, as needed. All procedures that can cause infection from aerosols or splashes are performed within a biological safety cabinet (BSC). An autoclave or an alternative method of decontamination is available for proper disposals. The laboratory has self-closing, lockable doors. A sink and eyewash station should be readily available. Biohazard warning signs are clear and legible in locations throughout the laboratory as appropriate.

In embodiments of the present disclosure, stable cell clones (such as stable BKH-21 cell clones) harboring autonomously replicating SARS-CoV-2 RNAs without S, M, E (and in some examples also NP) genes. In some embodiments, a pair of mutations is introduced into the non-structural protein 1 (Nsp1) of the disclosed SARS-CoV-2 to ameliorate cellular toxicity associated with virus replication. These stable cell clones, which harbor autonomously replicating SARS-CoV-2 RNA without producing infectious virus, can be readily cultured in most industrial laboratory settings, including BSL-2 laboratory conditions, for example, for use in high-throughput testing of anti-viral (such as anti-coronavirus, such as anti-SARS-CoV-2) compounds. In certain disclosed embodiments, a host cell (such as a BHK-21 cell) comprising an isolated, non-native coronavirus genome as disclosed herein, such as a genome comprising a nucleic acid molecule comprising at least 80%, at least 85%, at least 90%, at least 92%, at least 95%, at least 96%, at least 97%, at least 98%, at least 99%, or 100% sequence identity to SEQ ID NO: 1, 2, 3, 4, 5, 6, 7, 8, 9, 10, 11, 12, or 13, can be cultured in a BSL-2 laboratory setting.

In some examples, the screening methods further include selecting compounds identified as having anti-viral activity (e.g., those that reduce expression of the reporter gene or production of a reporter gene product, such as a fluorophore). In some examples, the screening methods further include administering such selected compounds into a research animal, such as a research mammal, such as a rabbit, non-human primate, cat, dog, mouse, or rat. Such administration can be systemic or local, for example via injection (e.g., i.p., i.v., or i.m.), inhalation, or oral.

### Kits

Provided herein are kits useful for the various embodiments described herein. Kits may contain various materials and reagents (e.g., for practicing the methods described herein). For example, a kit may contain reagents including, without limitation, polynucleotides (e.g., non-native coronavirus genomes), cells (such as stable cell clones expressing a non-native coronavirus genome provided herien), cell transfection reagents, reagents and materials for purifying polynucleotides including lysis regents, cell culture media, serum, as well as other solutions or buffers useful in carrying out the assays and other methods provided herein. Kits may also include control samples, materials useful in the methods described herein, and containers (such as those made of plastic or glass), tubes (such as those made of plastic or glass), microtiter plates and the like in which assay reactions may be conducted. Kits may be packaged in containers, which may include compartments for receiving the contents of the kits, and can include instructions for conducting methods described herein or using the cells and non-native coronavirus genomes described herein. For example, a kit can include (1) one or more isolated, non-native coronavirus genomes as described herein (including one or more nucleic acid molecules including or consisting of any one of SEQ ID NOs: 1-13), and/or (2) host cells (such as BHK-21 cells), which may or may not be pre-transfected or transfected with an isolated, non-native coronavirus genome. In one example, such a kit can further include transfection reagents. In one example, such a kit can further include cell culture reagents, such as culture media (such as DMEM, RPMI, and the like), animal serum (such as FBS), and/or antibiotics. In one example, such a kit can further include a control test agent, such as an anti-viral agent, such as remdesivir.

The kit can include a container and a label or package insert on or associated with the container. The label or package insert typically can further include instructions for use of the nucleic acid molecules and/or cells provided with the kit, for example for use in the methods disclosed herein. The instructional materials may be written, in an electronic form, or may be visual (such as video files).

### Example 1

### Materials and Methods

This example provides materials and methods used to generate the data described in the Examples below.

Cell culture and reagents: The human kidney epithelial cell line Lenti-X 293T was from Takara. The human liver cell line Huh7.5.1 was provided by Dr. Francis Chisari (Scripps Research Institute). The baby hamster kidney fibroblast cell line BHK21 (CCL-10), African green monkey kidney epithelial cells (Vero E6; CRL-1586), Caco-2 (HTB-37), Calu-3 (HTB-55) and A549 (CCL-185) were from the American Type Culture Collection. A549-hACE2 (NR-53821) cells were obtained from BEI Resources. All cell lines were maintained in DMEM supplemented with 5% penicillin and streptomycin, and 10% fetal bovine serum (FBS) at 37° C. with 5% CO2. The SARS-CoV-2 Nucleocapsid antibody (40143-MM05) was from Sino Biological. The SARS-CoV-2 Nsp1 antibody (PA5-116941) was from Themo Fisher Scientific. The R-actin antibody (GTX109639) was from Gentex. Secondary antibodies were from LI-COR Bioscience. GC376 Sodium was from Aobious (AOB36447). Remdesivir was from MedChemExpress (HY-104077).

Plasmid Construction: Doxycycline-inducible expression of SARS-CoV-2 NP was established in Vero E6, Huh7.5.1, and BHK-21 using TripZ-NP plasmid. NP cDNA was subcloned into pTripZ (AgeI/MluI) using the following primers: TripZ-NPf: 5′-ATATAGACCGGTCCACCATGTCTGATAATGGACCCCA-3′ (SEQ ID NO: 18), TripZ-NPr: 5′-ATATAGACGCGTTTAGGCCTGAGTTGAGTCAG-3′ (SEQ ID NO: 19).

Production of SARS-CoV-2 reporter virus: SARS-CoV-2 recombinant virus was generated using a 7-plasmid reverse genetic system which was based on the virus strain (2019-nCoV/USA_WA1/2020) isolated from the first reported SARS-CoV-2 case in the U.S. (Xie et al., Cell Host Microbe 27:841-848 e843, 2020). The initial 7 plasmids were from Dr. P-Y Shi (UTMB). Upon receipt, fragment 4 was subsequently subcloned into a low-copy plasmid pSMART LCAmp (Lucigen) to increase stability. Standard molecular biology techniques were employed to create the SARS-CoV-2 nanoluciferase reporter virus. In vitro transcription and electroporation were carried following known procedures (Xie et al., Nat Protoc 16:1761-1784, 2021).

SARS-CoV-2 Replicon: The SARS-CoV-2-Rep-NanoLuc-Neo replicon was constructed based on the full-length SARS-CoV-2 cDNA infectious clone (Xie et al., Cell Host Microbe 27:841-848 e843, 2020) by replacing the S gene with a nano luciferase gene, and by replacing M and E genes with a neomycin phosphotransferase (Neo) gene. To introduce Nsp1 R124S/K125E, N128S/K129E and K164A/H165A mutations into the SARS-CoV-2-Rep-NanoLuc-Neo replicon, puc57-CoV2-F1 plasmids containing mutated Nsp1 were first created by using overlap PCR method with the following primers: M13F: GTAAAACGACGGCCAGT (SEQ ID NO: 20); R124S/K125Ef: caaggttcttcttTCGgagaacggtaataaaggagct (SEQ ID NO: 21); R124S/K125Er: ttattaccgttctcCGAaagaagaaccttgcggtaag (SEQ ID NO: 22); N128S/K129Ef: taagaacggtAGTGAGggagctggtggccatagtta (SEQ ID NO: 23); N128S/K129Er: caccagctccCTCACTaccgttcttacgaagaagaa (SEQ ID NO: 24); K164A/H165Af: aaaactggaacactGCcGCcagcagtggtgttacccgtga (SEQ ID NO: 25); K164A/H165Ar: gggtaacaccactgctgGCgGCagtgttccagttttcttgaa (SEQ ID NO: 26); NheIr: cacgagcagcctctgatgca (SEQ ID NO: 27).

PCR fragments were digested by BglII/NheI and ligated into BglII/NheI digested F1 plasmid. The resulting plasmids were validated by restriction enzyme digestion and Sanger sequencing. Assembly of the 7 plasmids into the replicon and in vitro transcription were performed following a published protocol (Xie et al., Nat Protoc 16:1761-1784, 2021).

RNA Electroporation: Forty-eight hours post doxycycline treatment, BHK-21-NP^{Dox-ON} cells were washed with phosphate buffered saline (PBS), trypsinized, and resuspended in complete growth medium. Cells were pelleted by centrifugation (1,000×g for 5 min at 4° C.), washed twice with ice-cold DMEM, and resuspended in ice-cold Gene Pulser Electroporation Buffer (Bio-Rad) at 1×10⁷ cells/ml. Cells (0.4 ml) were then mixed with 10 μg of replicon RNA and 2 μg NP RNA, placed into 4 mm gap electroporation cuvettes, and electroporated at 270 V, 100Ω, and 950 μF in a Gene Pulser Xcell Total System (Bio-Rad). To establish stable replicon cells, 200 μg/mL of G418 was added to the media between 24 and 48 hours following electroporation, after which culture medium was changed every 2 to 3 days. Three weeks after G418 selection, the resultant foci were counted. All cells were trypsinized and pooled together in a T-75 flask for expansion (Pool #1 and Pool #2). Limiting dilution was subsequently performed to derive single cell clones. Determination of cell doubling time: Stable replicon clones and the parent BHK-21-NP^{Dox-ON} cells (without doxycycline) were seeded in multiple 48-well plates in triplicates at a density of 1,000 cells per well. One plate was removed every 24 hours for fixation in 4% paraformaldehyde followed by Hoechst 33342 staining. Cell numbers were quantified on a BioTek Cytation 7 cell multimode reader. Doubling time was determined by GraphPad Prism 9.

Quantification of viral RNA copy number: Viral RNA was quantified by reverse-transcription quantitative PCR (RT-qPCR) on a StepOnePlus Real-Time PCR System (Applied Biosystems) using Luna Universal Probe One-Step RT-qPCR Kit (New England Biolabs) with an in-house developed protocol. Primers and probes for qPCR were as follows: ORF1ab forward: 5′-CCCTGTGGGTTTTACACTTAA-3′ (SEQ ID NO: 28), reverse: 5′-ACGATTGTGCATCAGCTGA-3′ (SEQ ID NO: 29), probe: FAM-CCGTCTGCGGTATGTGGAAAGGTTATGG (SEQ ID NO: 30)-BHQ1; NanoLuc gene subgenomic mRNA forward: 5′-CCAACCAACTTTCGATCTCTTG-3′ (SEQ ID NO: 31), reverse: 5′-GGACTTGGTCCAGGTTGTAG-3 (SEQ ID NO: 32), probe: FAM-ACGAACAATGGTCTTCACACTCGAAGA (SEQ ID NO: 33)-BHQ1; Neomycin phosphotransferase gene subgenomic mRNA forward: 5′-CGATCTCTTGTAGATCTGTTCTCTAAA-3′ (SEQ ID NO: 34), reverse: 5′-GCCCAGTCATAGCCGAATAG-3′ (SEQ ID NO: 35), probe: FAM-ACAAGATGGATTGCACGCAGGTTC (SEQ ID NO: 36)-BHQ1. To generate standard plasmids, the cDNAs of SARS-CoV-2 ORF1ab gene, NanoLuc gene sgmRNA and neomycin phosphotransferase gene sgmRNA were cloned into a pCR2.1-TOPO plasmid respectively. The copy number of replicon RNA was calculated by comparing to a standard curve obtained with serial dilutions of the standard plasmid.

Immunoblotting: Cells were grown in 24-well plates and lysates were prepared with RIPA buffer (50 mM Tris-HCl [pH 7.4]; 1% NP-40; 0.25% sodium deoxycholate; 150 mM NaCl; 1 mM EDTA; protease inhibitor cocktail (Sigma); 1 mM sodium orthovanadate), and insoluble material was precipitated by brief centrifugation. Lysates were loaded onto 4-20% SDSPAGE gels and transferred to a nitrocellulose membrane (LI-COR, Lincoln, NE), blocked with Intercept® (TBS) Blocking Buffer Tris-buffered saline blocking formulation ((LI-COR, Lincoln, NE) for 1 h, and incubated with the primary antibody overnight at 4° C. Membranes were blocked with Odyssey Blocking buffer (LI-COR, Lincoln, NE), followed by incubation with primary antibodies at 1:1000 dilutions. Membranes were washed three times with 1×TBS containing 0.05% Tween 20© polysorbate-type nonionic surfactant (v/v), incubated with IRDye secondary antibodies (LI-COR, Lincoln, NE) for 1 h, and washed again to remove unbound antibody. Odyssey CLx (LI-COR Biosystems, Lincoln, NE) was used to detect bound antibody complexes.

Compound Screen: 273 compounds (assembled by TargetMol) were diluted in culture media to a final concentration of 5 μM for initial screen. Approximately 1.5×10⁴ replicon cells/well were seeded in 96-well plates in the absence of G418. Twenty-four hours later, cell culture media (without G418) was replaced with media containing 5 μM of compounds or the same volume of diluent DMSO. After incubation at 37° C. for specified periods, cells were assayed for NanoLuc activity using Nano-Glo Luciferase Assay System (N1130, Promega) or cell viability using cellTiter-Glo (G7571, Promega).

For validation, A549-hACE2, Caco-2 or Calu-3 cells were seeded in 96-well plates at a density of 10⁴ cells/well. Twenty-four hours later, cells were infected with SARS2-NanoLuc reporter virus in triplicates at an MOI of 0.05 in culture medium containing compounds. After 24 hours at 37° C., cells were assayed for NanoLuc activity using Nano-Glo Luciferase Assay System or Luciferase Assay System (E1501, Promega).

Next-generation sequencing (NGS): To prepare sequencing libraries, 100 μl of total RNAs were extracted from 5×10⁵ replicon cells using RNeasy mini kit (Qiagen). RNA quality was assessed by Agilent 2100 Bioanalyzer (Agilent Technologies, Santa Clara, CA, USA), and the RNA integration numbers (RIN) were all greater than 9. A total of 300 ng of total RNA was used to prepare the sequencing library using Illumina Stranded Total RNA Prep, Ligation with Ribo-Zero Plus. After rRNA removal, adaptor ligation, and cDNA library concentration and normalization, prepared libraries were loaded onto a NextSeq sequencer (Illumnia, San Diego, CA) for deep sequencing of paired-end reads of 2×74 cycles. The numbers of reads mapped to the constructed virus genome range between 27,000 and 544,00 for individual samples. Variant calling was performed using Qiagen CLC Genomics Workbench V20 low-frequency variant detection with the requirement of significance of >5% and minimum frequency of >20%. For canonical sgmRNA identification, a set of six sequences were constructed based on the replicon genome, each consisting of 49 nucleotides upstream of the 6-bp transcription regulatory sequence (TRS) motif (ACGAAC) found in the leader sequence, the TRS-B sequence and 50 nucleotides downstream of each TRS-B, which extend into the coding region of each ORF. The specific sequences covering the junctions are shown in Table 1.

TABLE 1
Canonical Subgenomic mRNA (sgmRNA) sequences.

| Name of | |
|---|---|
| Subgenomic | |
| mRNA | Sequence of Subgenomic mRNA (listed as cDNA) |
| sgmNanoluc | CAGGTAACAAACCAACCAACTTTCGATCTCTTGTAGATCTGTTCT |
| (SEQ ID NO: 37) | CTAAacgaacaATGGTCTTCACACTCGAAGATTTCGTTGGGGACT |
| | GGCGACAGACAGCCGG (underlined, leader sequence; |
| | lowercase, TRS-B; bolded, NanoLuc) |
| sgmORF3a | CAGGTAACAAACCAACCAACTTTCGATCTCTTGTAGATCTGTTCT |
| (SEQ ID NO: 38) | CTAAacgaacttATGGATTTGTTTATGAGAATCTTCACAATTGGAA |
| | CTGTAACTTTGAAGCA (underlined, leader sequence; |
| | lowercase, TRS-B; bolded, ORF3a) |
| sgmNeoR | CAGGTAACAAACCAACCAACTTTCGATCTCTTGTAGATCTGTTCT |
| (SEQ ID NO: 39) | CTAAacgaacttATGATTGAACAAGATGGATTGCACGCAGGTTCT |
| | CCGGCCGCTTGGGTGGA (underlined, leader sequence; |
| | lowercase, TRS-B; bolded, NeoR) |
| sgmORF7 | CAGGTAACAAACCAACCAACTTTCGATCTCTTGTAGATCTGTTCT |
| (SEQ ID NO: 40) | CTAAACGacgaacATGAAAATTATTCTTTTCTTGGCACTGATAAC |
| | ACTCGCTACTTGTGAGCT (underlined, leader sequence; |
| | lowercase, TRS-B; bolded, ORF7) |
| sgmORF8 | CAGGTAACAAACCAACCAACTTTCGATCTCTTGTAGATCTGTTCT |
| (SEQ ID NO: 41) | CTAAacgaacATGAAATTTCTTGTTTTCTTAGGAATCATCACAAC |
| | TGTAGCTGCATTTCA (underlined, leader sequence; |
| | lowercase, TRS-B; bolded, ORF8) |
| sgmNP | CAGGTAACAAACCAACCAACTTTCGATCTCTTGTAGATCTGTTCT |
| (SEQ ID NO: 42) | CTAAacgaacaaactaaaATGTCTGATAATGGACCCCAAAATCAGCG |
| | AAATGCACCCCGCATTACGTT (underlined, leader sequence; |
| | lowercase, TRS-B; bolded, NP) |

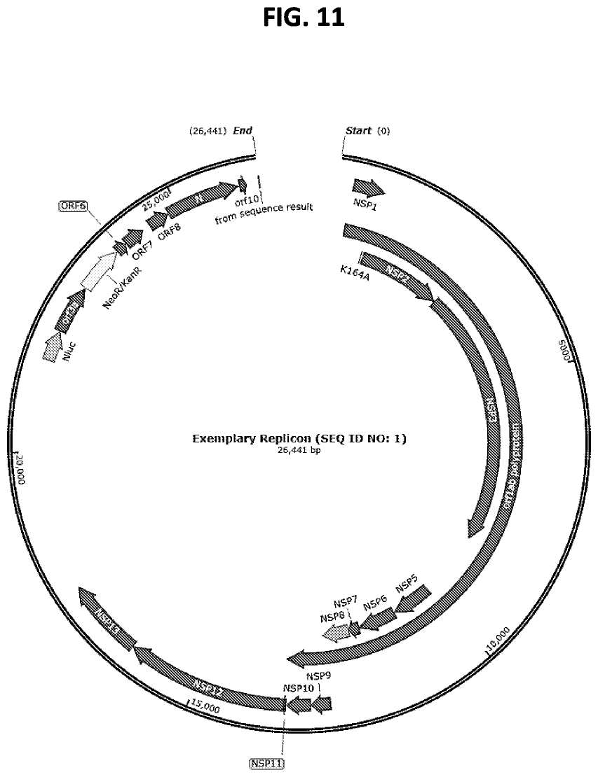

Short, paired-end reads of RNA samples from 12 stable replicon cell clones were uploaded and analyzed on the NGS platform high performance integrated virtual environment (HIVE). The reads were indexed, deduplicated, and quality metrics were collected upon data ingestion, which verified the high quality of the reads (FIGS. 11A-11D). Alignment of the reads were performed with HIVE's native against the seven reference sequences. HIVE Hexagon default parameters, tuned for viral analysis allowing for small indels and mutations, were used and a threshold of 65 bases or longer of the aligned query was applied. As the length of the reads was 70 bp, and due to the way the subject sequences were constructed, the alignments returned only split reads with patterns from both sides of the investigated junctions.

In Vitro Cytotoxicity Assay and CCso Determination: Cytotoxicity was determined by cell viability assay as previously described. In brief, the cell viability was measured using CellTiter Glo (Promega) according to the manufacturers' instructions, and luminescence signals were measured by GloMax luminometer. CCso values were calculated using a nonlinear regression curve fit in Prism Software version 9 (GraphPad). The reported CCso values were the results of at least 3 biological or technical replicates.

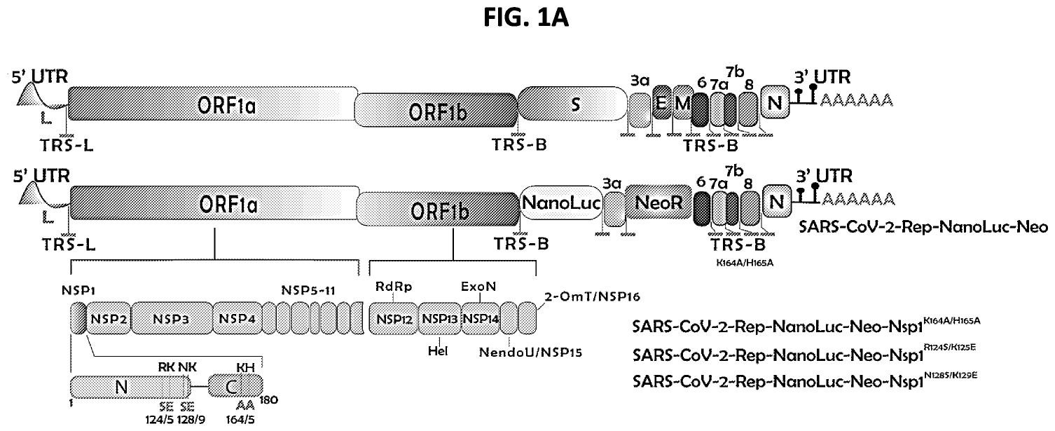

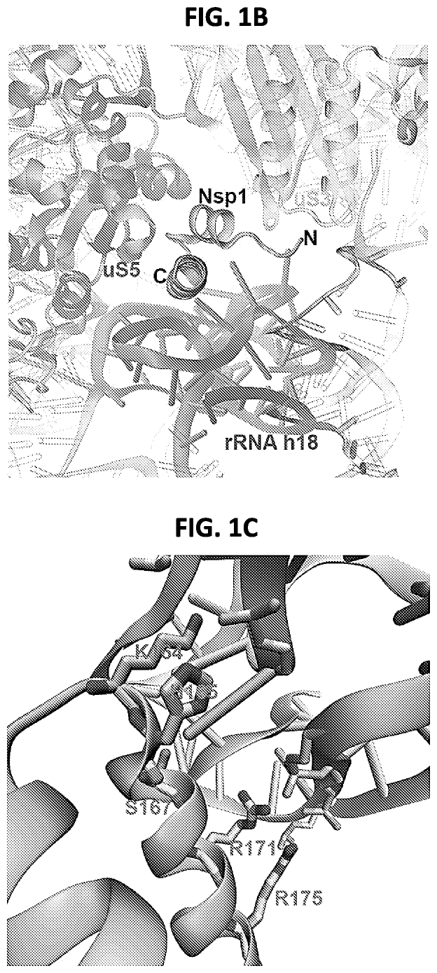

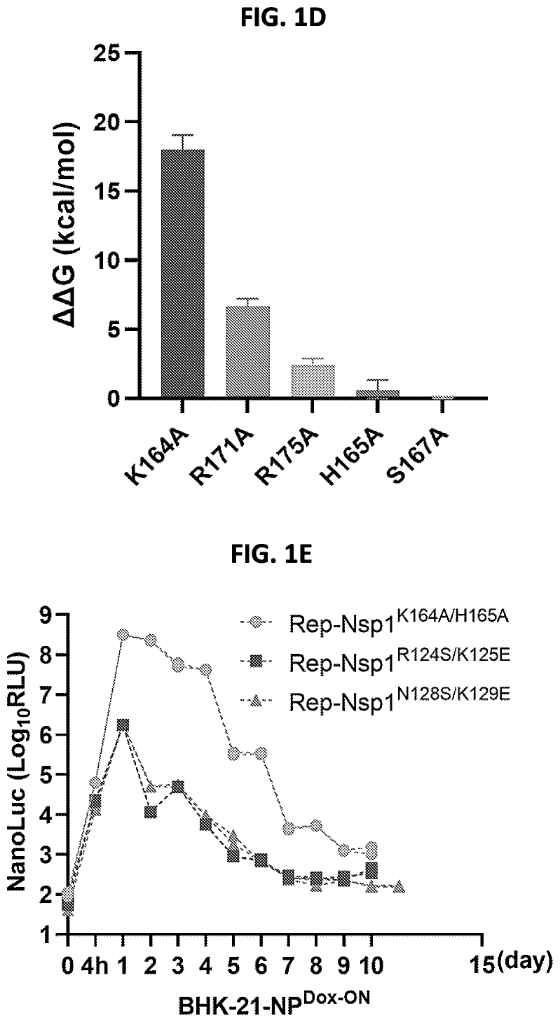

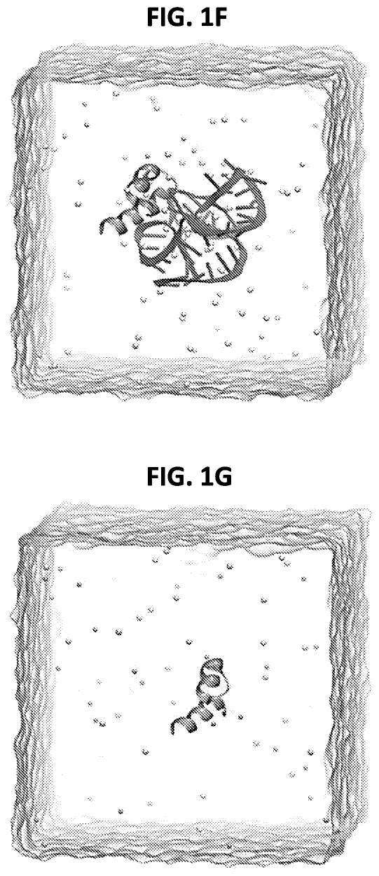

MD simulations: All-atom MD simulations were carried out for the complex of Nsp1 and the fragment of rRNA (charcoal gray in FIG. 1B) using the NAMD2.13 package (3) running on the IBM Power Cluster. The atomic coordinates for the complex (a bound state) were obtained from the crystal structure (PDB code: 7K5I) (Shi, et al. BioRxiv, 2020, doi:10.1101/2020.09.18.302901). The complex was further solvated in a cubic water box that measures about 78×78×78 Å³. Na⁺ and Cl⁻ were added to neutralize the entire simulation system and set the ion concentration to be 0.15 M. The final simulation system comprises 47,971 atoms. The built system was first minimized for 10 ps and then equilibrated for 1000 ps in the NPT ensemble (P˜1 bar and T˜300 K), with atoms in the backbones (of both nsp1 and RNA) harmonically restrained (spring constant k=1 kcal/mol/Å²). The production run (˜200 ns) was performed in the NPT ensemble, when only constraining the terminals of nsp1 (both N- and C-terminals) and rRNA (both 5′ and 3′-terminals). The same approach was applied in the production run for nsp1 in a 0.15 M NaCl electrolyte, a free state required in the free energy perturbation (FEP) calculations (see below). The water box for the nsp1-only simulation also measures about 78×78×78 Å (Huang et al., Nat Methods 14:71-73, 2017). Note that the similar system size for the bound and free states are required for free energy perturbation calculations for mutations with a net charge change.

The CHARMM36m force field (Huang et al., Nat Methods 14:71-73, 2017) was used for proteins and rRNA, the TIP3P model for water (Jorgensen, et al. J. Chem. Phys. 79:926, 1983; Neria, et al. J. Chem. Phys. 105:1902-1921, 1996), and the standard force field (Beglov, et al. J. Chem. Phys. 100:9050-9063, 1994) for Na⁺ and Cl⁻. The periodic boundary conditions (PBC) were applied in all three dimensions. Long-range Coulomb interactions were computed using particle-mesh Ewald (PME) full electrostatics with the grid size of about 1 Å in each dimension. The pair-wise van der Waals (vdW) energies were calculated using a smooth (10-12 Å) cutoff. The temperature T was kept at 300 K by applying the Langevin thermostat (Allen, D. J, Computer Simulation of Liquids. Oxford University Press: New York, 1987), while the pressure was maintained constant at 1 bar using the Nosé-Hoover method (Martyna, et al. J. Chem. Phys 101:4177-4189, 1994). With the SETTLE algorithm (Miyamoto. J. Comp. Chem 13:952-962, 1992) enabled to keep all bonds rigid, the simulation time-step was 2 fs for bonded and non-bonded (including vdW, angle, improper and dihedral) interactions, and the time-step for Coulomb interactions was 4 fs, with the multiple time-step algorithm (Tuckerman, et al. J. Chem. Phys 97:1990-2001, 1992).

Free energy perturbation calculations: After equilibrating the structures in bound and free states, free energy perturbation (FEP) calculations were performed (Chipot, A. Free energy calculations. Springer, 2007). In the perturbation method, many intermediate stages (denoted by u) whose Hamiltonian H(λ)=λH_{f}+(1−λ)Hᵢ are inserted between the initial (Hᵢ) and final (H_{f}) states to yield a high accuracy. With the softcore potential enabled, λ in each FEP calculation for the bound or free state varies from 0 to 1.0 in 20 perturbation windows (lasting 300 ps in each window), yielding gradual and progressive annihilation and exnihilation processes for mutations at residue 164 (K to A), 165 (H to A), 167 (S to A), 171 (R to A) and 175 (R to A), respectively. In FEP runs for the K164A mutation, the net charge of the MD system changed from 0 to −1 e (where e is the elementary charge). It is important to have similar sizes of the simulation systems for the free and the bound states (Gerhard & Garcia, J Phys Chem. 100:1206-1215, 1996; Luan, et al. J Phys Chem Lett. 7:2434-2438, 2016) so that the energy shifts from the Ewald summation (due to the net charge in the final simulation system) approximately cancel out when calculating ΔΔG. The same approaches were applied to investigate mutations of R171A and R175A. More detailed procedures can be found in previous work (Luan et al. J Phys Chem Lett. 11:9781-9787, 2020; Luan et al. J Med Chem. 2021, doi:10.1021/acs.jmedchem.1c00311).

Immunofluorescence: Stable cells were plated on collagen-coated glass coverslips into a 24-well plate at 1×10⁴ cells/well 2 days before fixation. The cells were washed for 5 min three times with 1× phosphate-buffered saline. After wash, cells were fixed in 4% paraformaldehyde for 15 min and then permeabilized in on 0.2% Triton X-100© nonionic surfactant at room temperature. An anti-dsRNA antibody (Kerafast, rJ2) was added at 1:40 for overnight at 4° C. followed by incubation with 1:5,000 Alexa 568 conjugated goat anti-mouse antibody. Images were captured by a Leica STELLARIS laser scanning confocal microscope.

### Example 2

### SARS-CoV-2-Rep-NanoLuc-Neo Expression in Four Cell Lines

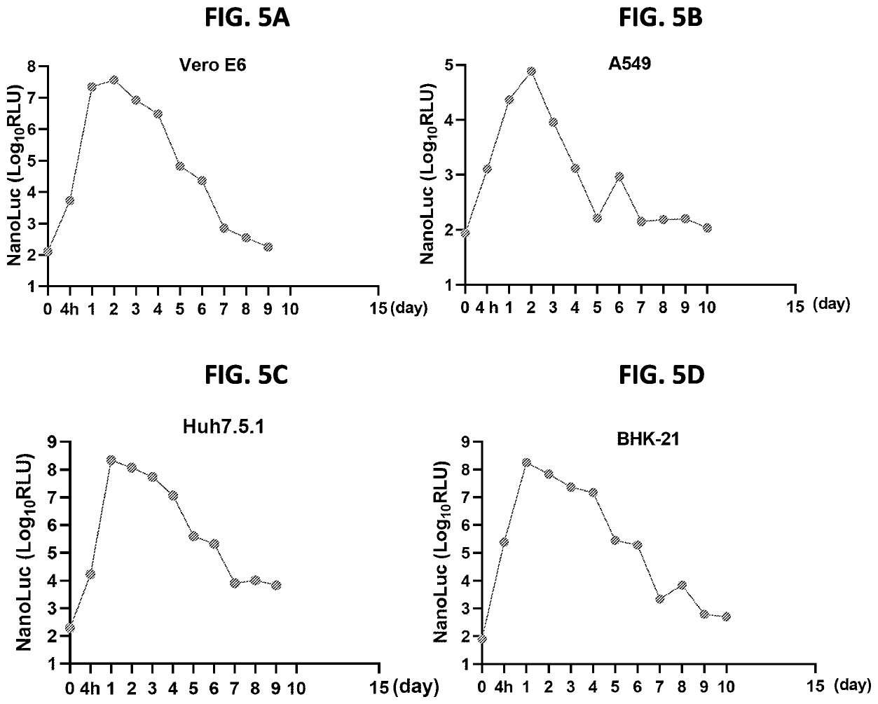

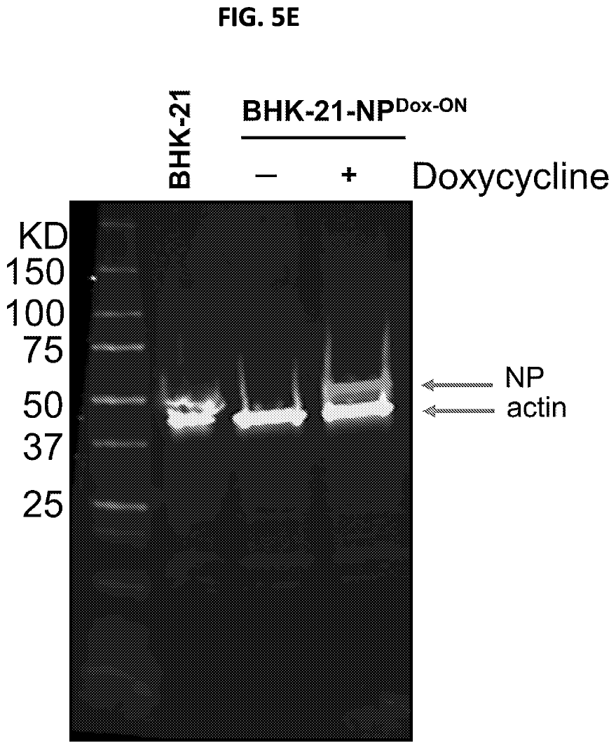

To generate stable cell clones harboring replicating SARS-CoV-2 RNAs, a replicon termed SARS-CoV-2-Rep-NanoLuc-Neo was constructed, in which the S gene was replaced by a nanoluciferase reporter (NanoLuc) and the E and M genes replaced with the neomycin phosphotransferase gene (NeoR) (FIG. 1A) Electroporation of this replicon RNA together with in vitro transcribed RNA encoding the nucleocapsid protein (NP) into Vero E6, Huh7.5.1, A549 or BHK-21 cells resulted in expression of nanoluciferase to varying extents (FIGS. 5A-5E). However, no viable clones could be recovered after 21 days of selection in G418, indicating that active replication of the replicon RNA is either unsustainable or cytotoxic. Huh7.5.1 and BHK-21 cells supported higher levels of nanoluciferase expression than Vero E6 and A549 cells, although the differences could be attributed to different electroporation efficiency of the four cell lines.

### Example 3

### SARS-CoV-2-Rep-NanoLuc-Neo Expression in BHK-21-NP^{Dox-ON} Cells

Because electroporating two different RNA species into the same cell was inefficient, a BHK-21 stable cell clone (BHK-21-NP^{Dox-ON}) was created (SEQ ID NO: 15), in which NP (e.g., NP in FIG. 1A) was expressed in a doxycycline-inducible manner (FIG. 5E). Electroporation of SARS-CoV-2-Rep-NanoLuc-Neo RNA into BHK-21-NP^{Dox-ON} cells resulted in three neomycin-resistant clones out of 4 million cells. The resultant clones grew very slow in the presence of 200 μg/mL G418 and no nanoluciferase activity or viral RNA was detected, indicating the loss of full-length replicon RNA. Although SARS-CoV-2-Rep-NanoLuc-Neo is selectable, persistent replication of this replicon could not be achieved in any of the four mammalian cell lines.

### Example 4

SARS-CoV-2-Rep-NanoLuc-Neo Replicons with Nsp1 Mutations Reduced Cytotoxicity Coronaviruses have evolved a variety of mechanisms to shut off host transcription and translation (Finkel et al., Nature 594, 240-245 (2021); Kamitani et al., Nat Struct Mol Biol 16, 1134-1140 (2009); Lokugamage et al., J Virol 89, 10970-10981 (2015)). Of all SARS-CoV-2 proteins, Nsp1 caused the most severe viability reduction in cells of human lung origin (Yuan et al., Mol Cell 80, 1055-1066 e1056 (2020)). The carboxyl terminus of Nsp1 folds into two helices which insert into the mRNA entrance channel on the 40S ribosome subunit, preventing both the host mRNA and viral mRNA from getting access to ribosomes and consequently shutting down translation (Schubert et al., Nat Struct Mol Biol 27, 959-966 (2020); Lapointe et al., Proc Natl Acad Sci USA 118(6):e2017715118 (2021)) (FIG. 1B). The first C-terminal helix (residues 153-160) makes hydrophobic interactions with 40S ribosomal proteins uS3 and uSS, and the second C-terminal helix (residues 166-178) interacts with ribosomal protein eS30 and with the phosphate backbone of h18 of the 18S rRNA via the two conserved arginines R171 and R175 (Schubert et al., Nat Struct Mol Biol 27, 959-966 (2020)). In between the two helices, a conserved KH dipeptide (K164 and H165) forms critical interactions with h18 that are based on H165 stacking between two uridines of 18S rRNA (U607 and U630), and electrostatic interactions between K164 and the phosphate backbone of rRNA bases G625 and U630 (FIG. 1C). Free energy perturbation calculations revealed that mutations of residues K164, R171, R175, H165, and S167 of Nsp1 to alanine will reduce the interaction in the order of impact (FIGS. 1D, 1F, and 1G).

It was hypothesized that a pair of mutations (such as K164A/H165A) that weaken the interaction between C-terminus of Nsp1 and ribosome will lead to a shorter occupation time of Nsp1 on ribosome and the accessibility of ribosomes to host mRNA. As a result, the Nsp1-mediated toxicity to the host will be alleviated. To test this possibility, a new replicon construct SARS-CoV-2-Rep-NanoLuc-Neo-Nsp1^{K164A/H165A} (SEQ ID NO: 1) was created, in which K164/H165 were mutated to alanine (FIG. 1A). For comparison, two other replicons were made, SARS-CoV-2-Rep-NanoLuc-Neo-Nsp1^{R124S/K125E} (SEQ ID NO: 16) and SARS-CoV-2-Rep-NanoLuc-Neo-Nsp1^{N128/K129E} (SEQ ID NO: 17), given that both pairs of mutations (R124S/K125E and N128S/K129E) reportedly reduced Nsp1-mediated cell toxicity in a human lung cell line (Yuan et al., Mol Cell 80, 1055-1066 e1056 (2020)).

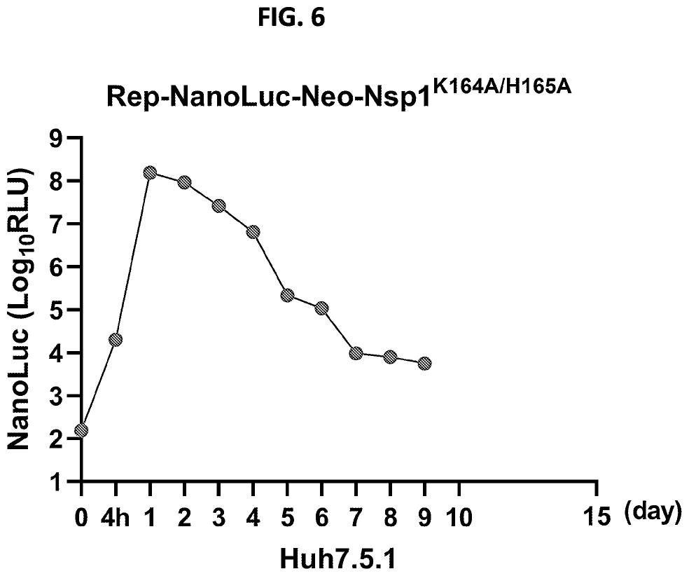

When electroporated into BHK-21-NP^{Dox-ON} cells, all three replicons led to transient reporter gene expression (FIG. 1E). However, in the presence of 200 μg/mL G418, only SARS-CoV-2-Rep-NanoLuc-Neo-Nsp1^{K164A/H165A} yielded viable cells (Pool #1, SEQ ID NO: 1), from which 12 stable clones (Clone #2-13, SEQ ID NOS: 2-13) were subsequently derived by limiting dilution. Electroporation of replicon RNA was independently performed three times and each time only SARS-CoV-2-Rep-NanoLuc-Neo-Nsp1^{K64A/H165A} yielded viable clones in BHK-21-NP^{Dox-ON} cells (Table 2). SARS-CoV-2-Rep-NanoLuc-Neo-Nsp1^{K164A/H165A} also replicated well in Huh7.5.1 cell line although no viable cells could be recovered after G418 selection (FIG. 6). Moreover, at least 4 clones were derived using standard BHK-21 cells, albeit at lower efficiency.

TABLE 2
Viable clone numbers.

| | 1^{st} | 2^{nd} | 3^{rd} |
|---|---|---|---|
| | transfection | transfection | transfection |
| SARS-CoV-2-Rep-NanoLuc- | 3*(0) | 0 | 0 |
| Neo | | | |
| SARS-CoV-2-Rep-NanoLuc- | 132 | 7 | 107 |
| Neo-Nsp1^{K164A/H165A} | | | |

*Three clones were initially obtained after G418 selection but were devoid of the replicon RNA after sequencing. 4 × 10⁶ cells were electroporated with 10 μg of each RNA for each transfection.

### Example 5

### SARS-CoV-2-Rep-NanoLuc-Neo-Nsp1^{K164A/H165A} Expression in BHK-21-NP^{Dox-ON} Cells without Selection Pressure

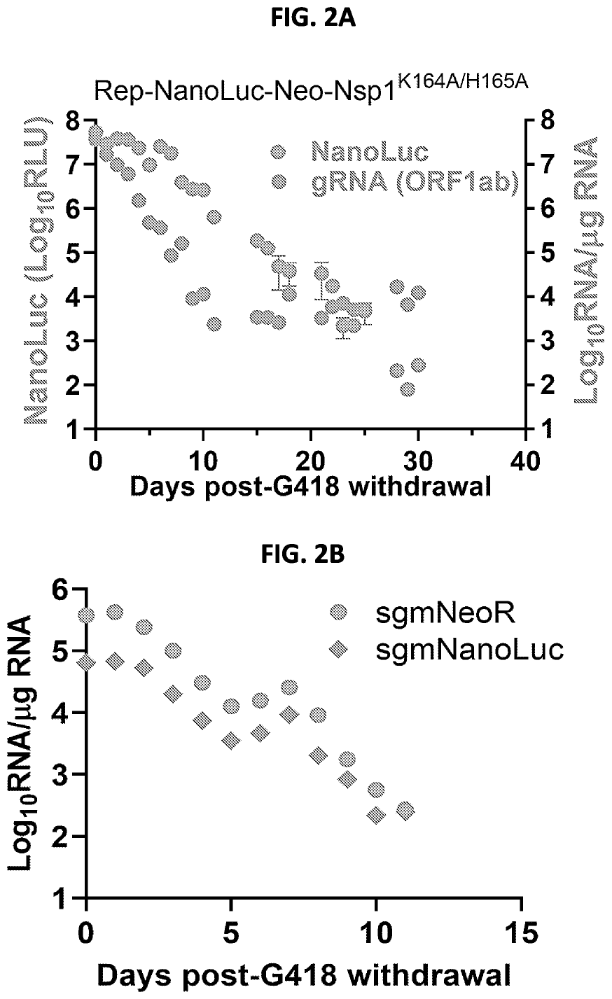

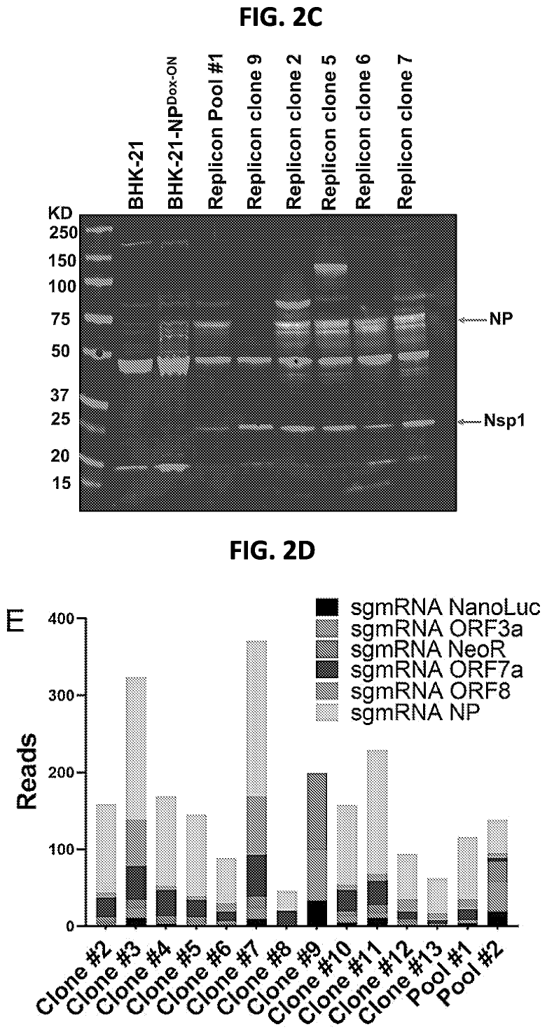

To determine if SARS-CoV-2-Rep-NanoLuc-Neo-Nsp1^{K164A/H165A} could persist in cells without selection pressure, G418 was subsequently withdrawn from Pool #1 cells after the initial selection. For up to one week, there was no significant loss of nanoluciferase expression, a feature that is highly desirable in drug screens. The level of nanoluciferase decreased by one log after 10 days of culturing without G418, and two logs after 21 days (FIG. 2A).

### Example 6

### Profiling gRNA and sgmRNA Species in Cells Harboring SARS-CoV-2-Rep-NanoLuc-Neo-Nsp1^{K164A/H165A}

Quantitative reverse transcription PCR (RT-qPCR) was performed to profile gRNA and sgmRNA species in cells harboring SARS-CoV-2-Rep-NanoLuc-Neo-Nsp1^{K164A/H165A} (stable cell clones 2, 5, 6, 7, and 9; SEQ ID NOs: 2, 5, 6, 7, and 9). SARS-CoV-2 transcribes multiple canonical sgmRNAs, including S, E, M, NP, ORF3a, ORF6, ORF7a, ORF7b, ORF8 and ORF10, although multiple studies have found negligible ORF10 expression and very few ORF7b body-leader junctions (Kim et al., Cell 181, 914-921 e910 (2020); Finkel et al., Nature 589, 125-130 (2021)). In the disclosed replicon cells, S, M, E sgmRNA are replaced with that encoding NanoLuc and NeoR. sgmRNA encoding ORF6 is lost because its transcriptional regulatory sequence body (TRS-B) resides in the M gene, which is also deleted in the replicon RNA. Hence, cells harboring the replicon would express at least six sgmRNAs, namely Nanoluc, NeoR, ORF3a, ORF7a, ORF8, and NP.

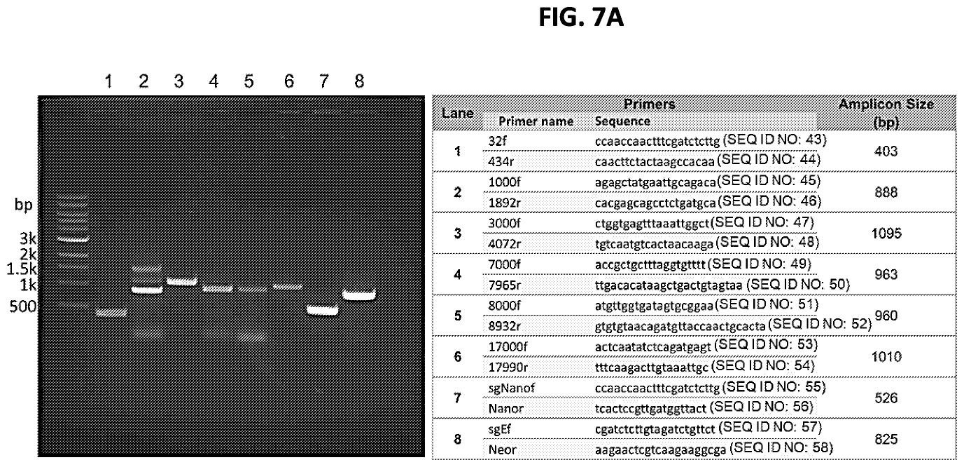

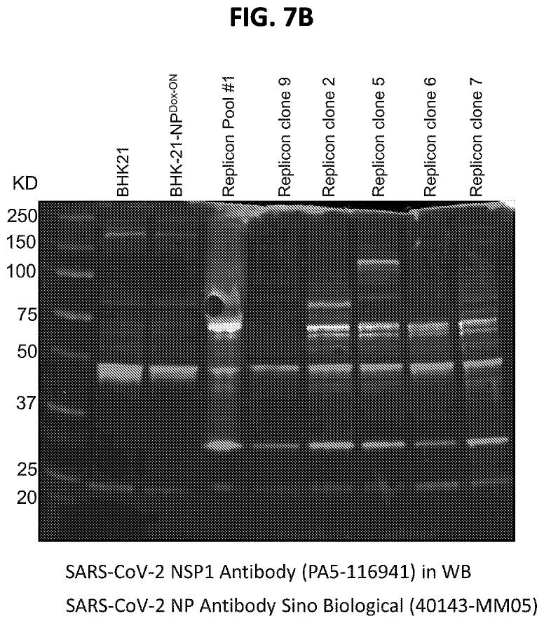

Primers and probes were designed to specifically amplify the gRNA of the replicon and sgmRNAs of NanoLuc and NeoR. Temporal expression of gRNA and sgmRNA for NanoLuc and NeoR gene was clearly observed in Pool #1 cells (FIG. 2B and FIG. 7A). Western blotting further confirmed the presence of Nsp1 and NP in replicon cell lysates (FIG. 2C and FIG. 7B).

### Example 7

### Sequencing of SARS-CoV-2-Rep-NanoLuc-Neo-Nsp1^{K164A/H165A} in Stable Clones

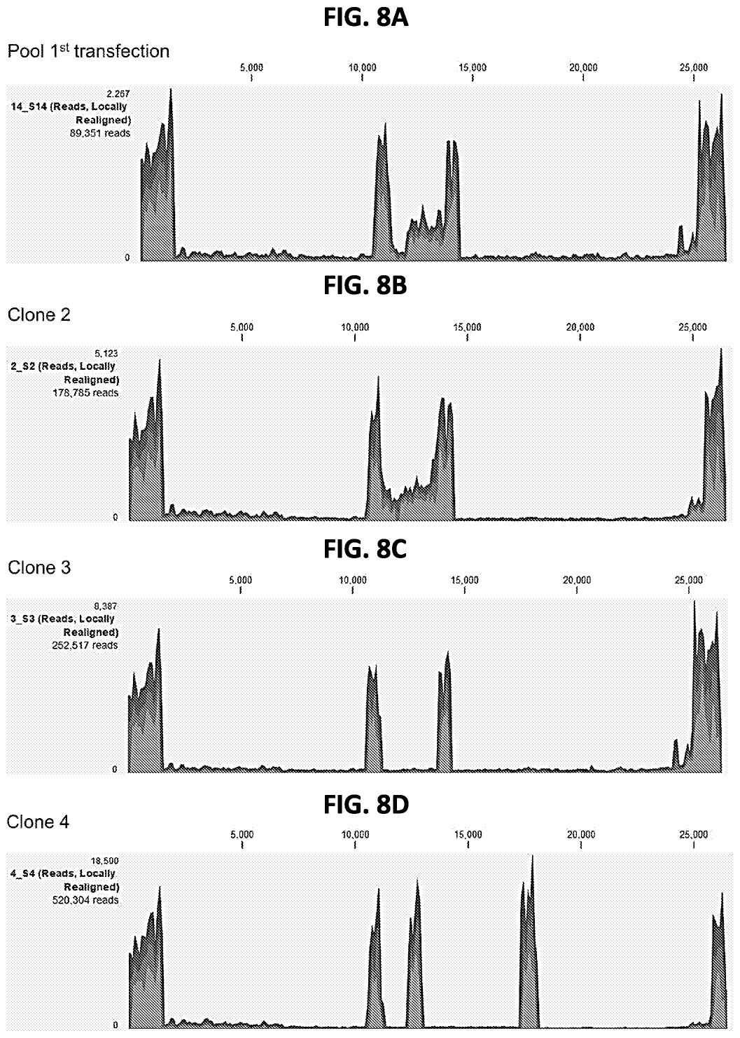

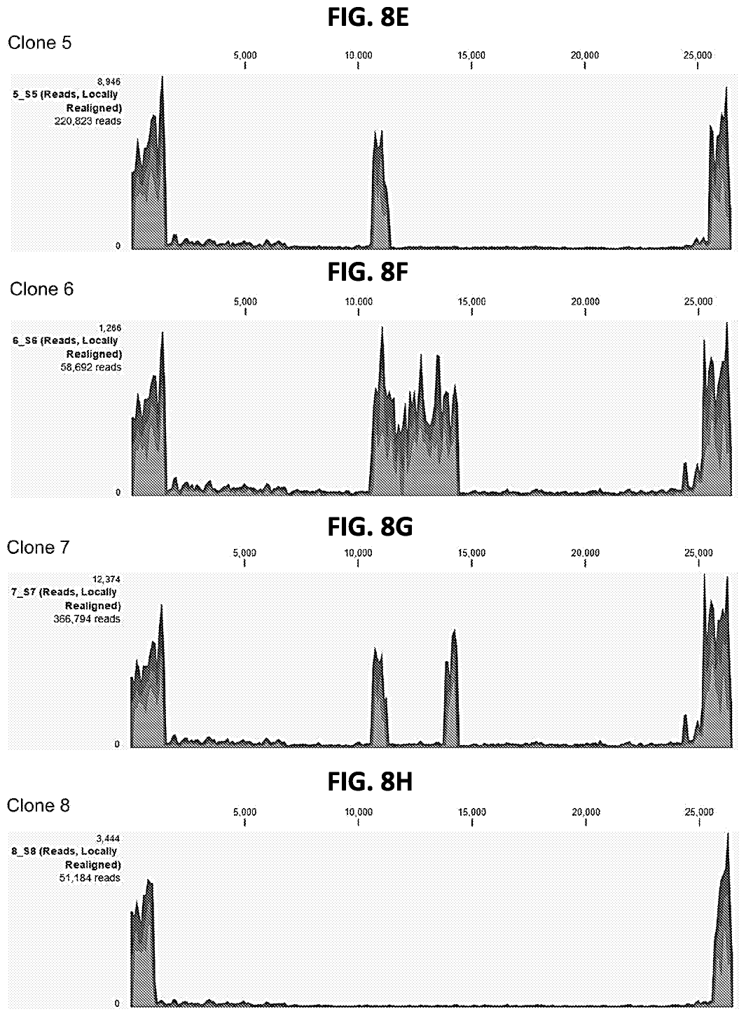

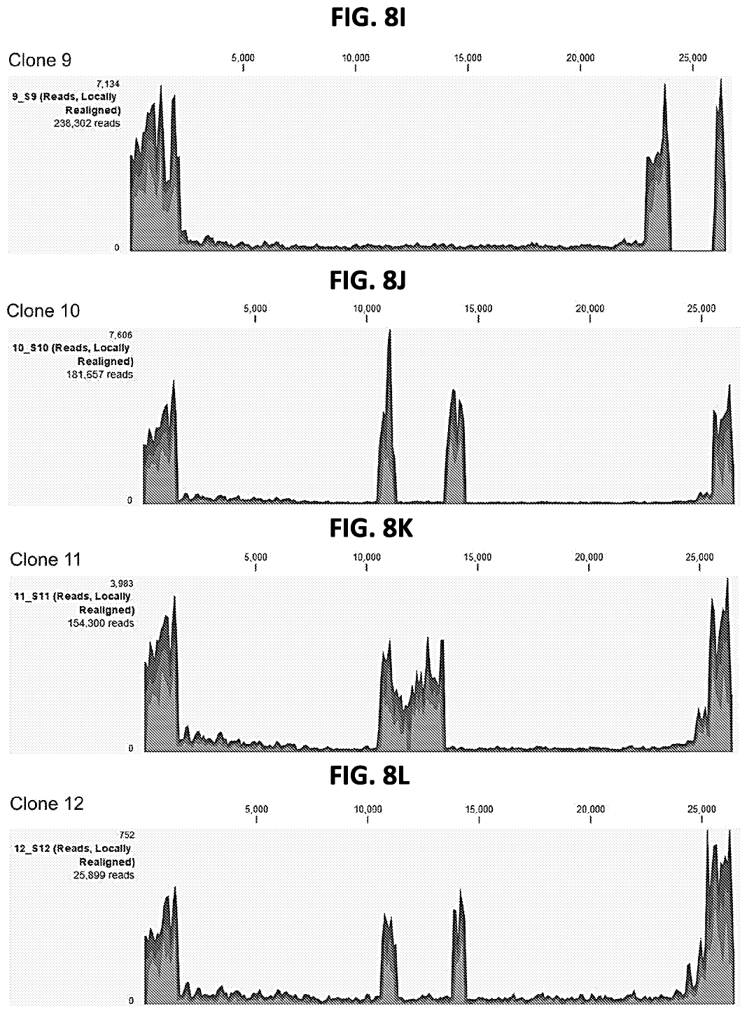

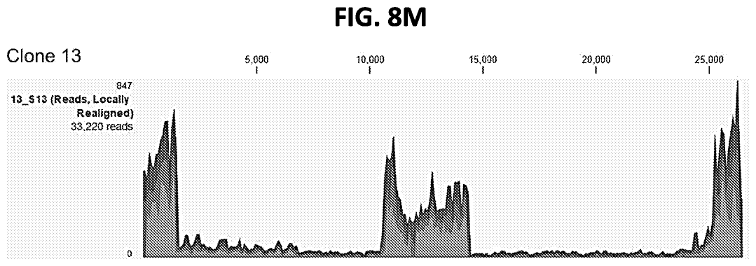

To further characterize the replicon gRNA after extended drug selection, next-generation sequencing (NGS) was performed on all 12 stable clones. While the replicon gRNA containing Nsp1^{K164A/H165A} was present in all clones, additional synonymous or missense mutations were detected in each clone (FIGS. 8A-8M, Table 3; also see SEQ ID NOS: 2-13). A Nsp4 R401S substitution was detected in 10 out of 12 clones and a Nsp10 Ti111I substitution appeared in 6 of 12 clones. Such mutations may enhance replicon replication efficiency in BHK-21 cells.

Further, sequencing the replicon gRNA in clone #9 (SEQ ID NO: 9) also revealed a deletion knocking out ORF7a/b, ORF8, and the first 392 amino acids of the NP (FIG. 8I), indicating that NP is not required for virus replication. The presence of canonical sgmRNA species in each stable clone was also confirmed by NGS, although the method employed could not quantitively assess the abundance of each sgmRNA in a stable cell clone due to uneven coverage of sequence reads over different regions (FIG. 2D).

TABLE 3
Summary of NGS results of 12 stable cell clones harboring
SARS-COV-2-Rep-NanoLuc-Neo-Nsp1K164A/H165A

| | Position in | | Reported | | | | |
|---|---|---|---|---|---|---|---|
| | MN985325.1 | Position | MN985325.1 | | | | |
| | 2019-nCoV/ | in | 2019-nCoV/ | Reported | | | |
| | USA_WA1/ | Replicon | USA_WA1/ | Replicon | Identified | | |
| Clone | 2020 | Reference | 2020 | Reference | Alternative | | |
| number | Sequence | Sequence | Sequence | Sequence | Base | Amino Acid Change | Frequency |
| Clone 2 | 755-760 | 755-760 | AAACAT | AAACAT | GCCGCC | NSP1 K164A H165A | 96.52 |
| (SEQ ID | 7486* | 7486 | A | T | T | NSP3 S1589 Synonymous | 100 |
| NO: 2) | 7489* | 7489 | T | A | A | NSP3 | 100 |
| | | | | | | T1590 Synonymous | |
| | 6525 | 6525 | C | C | T | NSP3 T1269I | 71.68 |
| | 9755 | 9755 | C | C | A | NSP4 R401S | 100 |
| | 12786 | 12786 | C | C | T | NSP9 T34I | 40.04 |
| | 14724 | 14724 | C | C | T | Synonymous | 100 |
| | 21599-25381 | Deleted | S gene | Nano-Luc | Nano-Luc | S is replaced with NanoLuc | 100 |
| | 26248-27190 | | E and M Gene | NeoR | NeoR | E and M are replaced with | 100 |
| | | | | | | neomycin resistant gene | |
| | | | | | | (NeoR) | |
| Clone 3 | 755-760 | 755-760 | AAACAT | AAACAT | GCCGCC | NSP1 K164A H165A | 97.25 |
| (SEQ ID | 7486* | 7486 | A | T | T | NSP3 S1589 Synonymous | 100 |
| NO: 3) | 7489* | 7489 | T | A | A | NSP3 T1590 Synonymous | 100 |
| | 9755 | 9755 | C | C | A | NSP4 R401S | 100 |
| | 13356 | 13356 | C | C | T | NSP10 T111I | 100 |
| | 21599-25381 | Deleted | S gene | Nano-Luc | Nano-Luc | S is replaced with NanoLuc | 100 |
| | 26248-27190 | | E and M Gene | NeoR | NeoR | E and M are replaced with | 100 |
| | | | | | | NeoR | |
| Clone 4 | 755-760 | 755-760 | AAACAT | AAACAT | GCCGCC | NSP1 K164A H165A | 97.25 |
| (SEQ ID | 9755 | 9755 | C | C | A | NSP4 R401S | 100 |
| NO: 4) | 7486* | 7486 | A | T | T | NSP3 S1589 Synonymous | 100 |
| | 7489* | 7489 | T | A | A | NSP3 T1590 Synonymous | 99.42 |
| | 19722 | 19722 | A | A | C | NSP15 K34N | 75.36 |
| | N/A | 21788-21789 | N/A | Insertion | A | Nanoluc E76 | 66.06 |
| | N/A | 21833 | N/A | A | G | Nanoluc K91E | 99.09 |
| | N/A | 21862-21863 | N/A | AA | GG | Nanoluc V100 synonymous | 100 |
| | | | | | | I101V | |
| | 21599-25381 | Deleted | S gene | Nano-Luc | Nano-Luc | S is replaced with NanoLuc | 100 |
| | 26248-27190 | | E and M Gene | NeoR | NeoR | E and M are replaced with | 100 |
| | | | | | | NeoR | |
| | 28955 | 25497 | A | A | G | NP N228D | 97.46 |
| | 29446 | 25988 | T | T | GA | NP T391 | 90.27 |
| | 29449 | 25991 | G | G | A | NP V392 | 92.21 |
| Clone 5 | 755-760 | 755-760 | AAACAT | AAACAT | GCCGCC | NSP1 K164A H165A | 96.39 |
| (SEQ ID | 7486* | 7486 | A | T | T | NSP3 S1589 Synonymous | 100 |
| NO: 5) | 7489* | 7489 | T | A | A | NSP3 T1590 Synonymous | 100 |
| | 9755 | 9755 | C | C | A | NSP4 R401S | 100 |
| | 15006 | 15006 | G | G | T | NSP12 D523Y | 100 |
| | N/A | 21833 | N/A | A | G | Nanoluc K91E | 100 |
| | N/A | 21862-21863 | N/A | AA | GG | Nanoluc | 100 |
| | | | | | | V100 synonymous | |
| | | | | | | I101V | |
| | 21599-25381 | Deleted | S gene | Nano-Luc | Nano-Luc | S is replaced with NanoLuc | 100 |
| | 26248-27190 | | E and M Gene | NeoR | NeoR | E and M are replaced with | 100 |
| | | | | | | NeoR | |
| | 26078 | 22772 | C | C | T | ORF3a T229I | 100 |
| | 28955 | 25497 | A | A | G | NP N228D | 98.41 |
| | 29448 | 25990 | T | T | A | NP V392E | 78.44 |
| Clone 6 | 755-760 | 755-760 | AAACAT | AAACAT | GCCGCC | NSP1 K164A H165A | 97.46 |
| (SEQ ID | 7486* | 7486 | A | T | T | NSP3 S1589 Synonymous | 100 |
| NO: 6) | 7489* | 7489 | T | A | A | NSP3 T1590 Synonymous | 100 |
| | 11075 | 11075 | Deletion T | Deletion | | NSP6 | 38.16 |
| | | | | T | | | |
| | 15221 | 15221 | T | T | G | NSP12 F594C | 100 |
| | 21599-25381 | Deleted | S gene | Nano-Luc | Nano-Luc | S is replaced with NanoLuc | 100 |
| | 26248-27190 | | E and M Gene | NeoR | NeoR | E and M are replaced with | 100 |
| | | | | | | NeoR | |
| Clone 7 | 755-760 | 755-760 | AAACAT | AAACAT | GCCGCC | NSP1 K164A H165A | 96.83 |
| (SEQ ID | 2189 | 2189 | C | C | T | NSP2 L462F | 95.45 |
| NO: 7) | 7486* | 7486 | A | T | T | NSP3 S1589 Synonymous | 99.45 |
| | 7489* | 7489 | T | A | A | NSP3 T1590 Synonymous | 100 |
| | 9755 | 9755 | C | C | A | NSP4 R401S | 99.17 |
| | 12357 | 12357 | C | C | T | NSP8 T89I | 100 |
| | 13356 | 13356 | C | C | T | NSP10 T111I | 100 |
| | 13953 | 13953 | A | A | C | NSP 12 1171 Synonymous | 94.36 |
| | 21599-25381 | Deleted | S gene | Nano-Luc | Nano-Luc | S is replaced with NanoLuc | 100 |
| | 26248-27190 | | E and M Gene | NeoR | NeoR | E and M are replaced with | 100 |
| | | | | | | NeoR | |
| | N/A | 23478 | N/A | C | T | NeoR synonymous | 100 |
| Clone 8 | 755-760 | 755-760 | AAACAT | AAACAT | GCCGCC | NSP1 K164A H165A | 97.57 |
| (SEQ ID | 1392 | 1392 | C | C | T | NSP2 S196L | 100 |
| NO: 8) | 7486* | 7486 | A | T | T | NSP3 S1589 Synonymous | 100 |
| | 7489* | 7489 | T | A | A | NSP3 T1590 Synonymous | 95.65 |
| | 9755 | 9755 | C | C | A | NSP4 R401S | 90 |
| | 13356 | 13356 | C | C | T | NSP10 T111I | 94.44 |
| | 15521 | 15521 | T | T | G | NSP12 F594C | 100 |
| | 21599-25381 | Deleted | S gene | Nano-Luc | Nano-Luc | S is replaced with NanoLuc | 100 |
| | 26248-27190 | | E and M Gene | NeoR | NeoR | E and M are replaced with | 100 |
| | | | | | | NeoR | |
| Clone 9 | 755-760 | 755-760 | AAACAT | AAACAT | GCCGCC | NSP1 K164A H165A | 97.64 |
| (SEQ ID | 2416 | 2416 | C | C | T | NSP2 Y537 synonymous | 99.79 |
| NO: 9) | 6896 | 6896 | C | C | T | NSP3 L1393 synonymous | 98.99 |
| | 7486* | 7486 | A | T | T | NSP3 S1589 Synonymous | 100 |
| | 7489* | 7489 | T | A | A | NSP3 T1590 Synonymous | 100 |
| | 9755 | 9755 | C | C | A | NSP4 R401S | 99.04 |
| | 11286 | 11286 | T | T | G | NSP6 L105W | 98.69 |
| | 13356 | 13356 | C | C | T | NSP10 T111I | 99.28 |
| | 21599-25381 | Deleted | S gene | Nano-Luc | Nano-Luc | S is replaced with NanoLuc | 100 |
| | 26248-27190 | | E and M Gene | NeoR | NeoR | E and M are replaced with | 100 |
| | | | | | | NeoR | |
| | 26132 | 22826 | A | A | C | ORF3A H246P | 100 |
| | 27392-29448 | 23934-25990 | ORF7-N | deletion | | Deletion ORF7-N | 100 |
| Clone | 755-760 | 755-760 | AAACAT | AAACAT | GCCGCC | NSP1 K164A H165A | 97.29 |
| 10 | 2751 | 2751 | C | C | T | NSP3 T11I | 100 |
| (SEQ ID | 7486* | 7486 | A | T | T | NSP3 S1589 Synonymous | 98.53 |
| NO: 10) | 7489* | 7489 | T | A | A | NSP3 | 98.59 |
| | | | | | | T1590 Synonymous | |
| | 8558 | 8558 | A | A | G | NSP4 I2V | 100 |
| | 9755 | 9755 | C | C | A | NSP4 R401S | 100 |
| | 13356 | 13356 | C | C | T | NSP10 T111I | 100 |
| | 21599-25381 | Deleted | S gene | Nano-Luc | Nano-Luc | S is replaced with NanoLuc | 100 |
| | 26248-27190 | | E and M Gene | NeoR | NeoR | E and M are replaced with | 100 |
| | | | | | | NeoR | |
| | N/A | 23190 | | C | T | NeoR V84 synonymous | 100 |
| | 27625 | 24167 | C | C | T | ORF7 R78C | 81.40 |
| Clone | 755-760 | 755-760 | AAACAT | AAACAT | GCCGCC | NSP1 K164A H165A | 97.87 |
| 11 | 7486* | 7486 | A | T | T | NSP3 S1589 Synonymous | 100 |
| (SEQ ID | 7489* | 7489 | T | A | A | NSP3 T1590 Synonymous | 100 |
| NO: 11) | 9755 | 9755 | C | C | A | NSP4 R401S | 100 |
| | 19102 | 19102 | C | C | T | NSP14 | 100 |
| | 21599-25381 | Deleted | S gene | Nano-Luc | Nano-Luc | S is replaced with NanoLuc | 100 |
| | 26248-27190 | | E and M Gene | NeoR | NeoR | E and M are replaced with | 100 |
| | | | | | | NeoR | |
| Clone | 755-760 | 755-760 | AAACAT | AAACAT | GCCGCC | NSP1 K164A H165A | 98.96 |
| 12 | 1182 | 1182 | T | T | C | NSP2 V126A | 20.42 |
| (SEQ ID | 2954 | 2954 | T | T | G | NSP3 L79V | 66.67 |
| NO: 12) | 7486* | 7486 | A | T | T | NSP3 S1589 Synonymous | 100 |
| | 7489* | 7489 | T | A | A | NSP3 T1590 Synonymous | 100 |
| | 9755 | 9755 | C | C | A | NSP4 R401S | 100 |
| | 11750 | 11750 | C | C | A | NSP6 L260I | 69.23 |
| | 12357 | 12357 | C | C | T | NSP8 T89I | 100 |
| | 13356 | 13356 | C | C | T | NSP10 T111I | 100 |
| | 13499 | 13499 | C | C | T | NSP12 T20I | 100 |
| | 13953 | 13953 | A | A | C | NSP 12 1171 Synonymous | 88.15 |
| | 21599-25381 | Deleted | S gene | Nano-Luc | Nano-Luc | S is replaced with NanoLuc | 100 |
| | 26248-27190 | | E and M Gene | NeoR | NeoR | E and M are replaced with | 100 |
| | | | | | | NeoR | |
| Clone | 755-760 | 755-760 | AAACAT | AAACAT | GCCGCC | NSP1 K164A H165A | 96.82 |
| 13 | 1392 | 1392 | C | C | T | NSP2 S196L | 24.01 |
| (SEQ ID | 7486* | 7486 | A | T | T | NSP3 S1589 Synonymous | 100 |
| NO: 13) | 7489* | 7489 | T | A | A | NSP3 T1590 Synonymous | 100 |
| | 11075 | 11075 | T deletion | T deletion | | NSP6 F35 | 23.86 |
| | 21599-25381 | Deleted | S gene | Nano-Luc | Nano-Luc | S is replaced with NanoLuc | 100 |
| | 26248-27190 | | E and M Gene | NeoR | NeoR | E and M are replaced with | 100 |
| | | | | | | NeoR | |

*These two mutations were introduced to differentiate the infectious clone-derived virus from the parental clinical isolate 2019-nCoV/USA_WA1/2020.

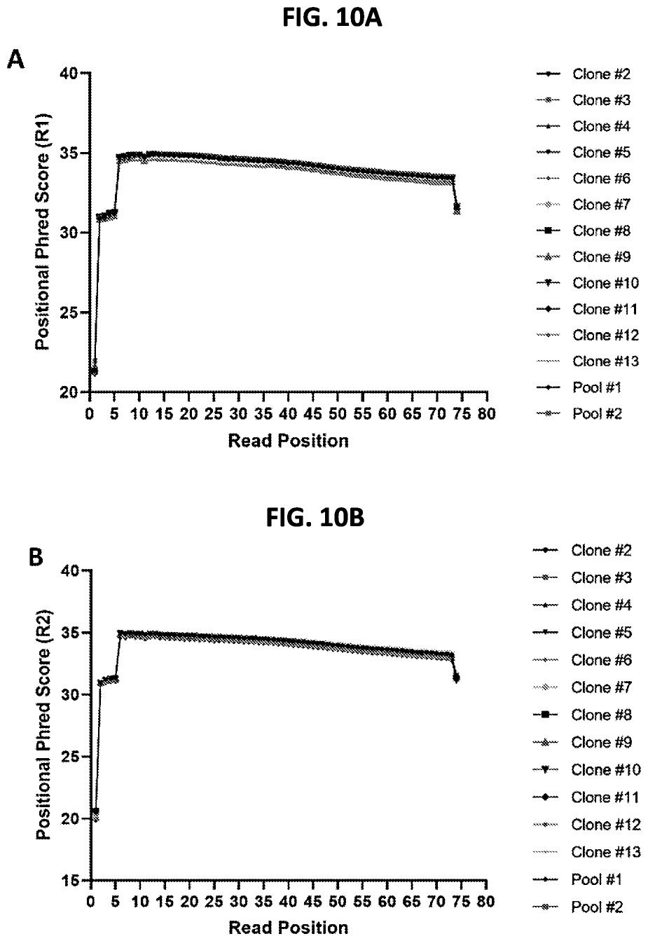

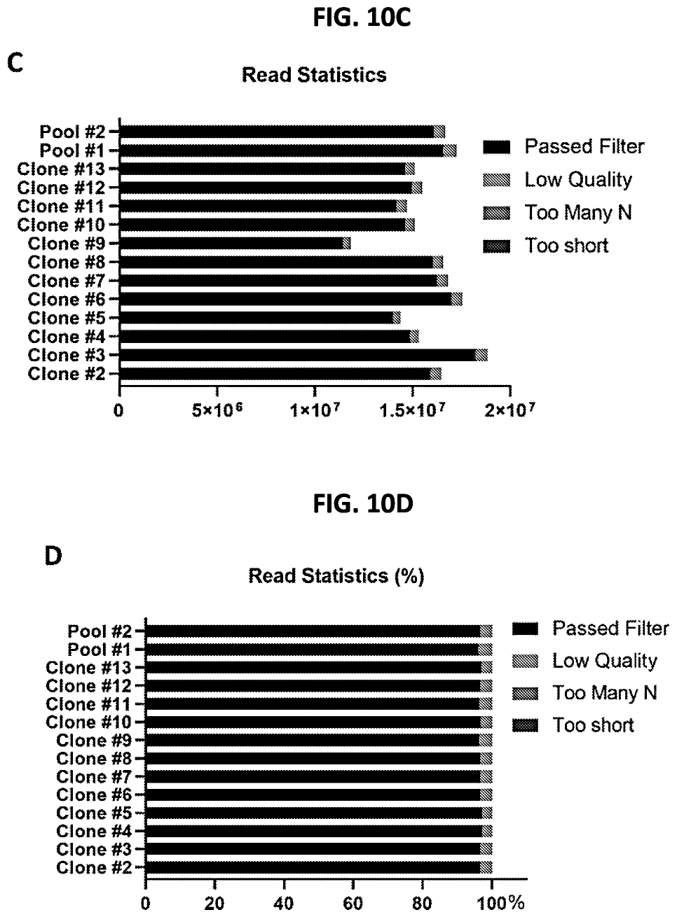

The 12 stable clones were also characterized for cellular morphology and growth kinetics. All stable cell clones maintained a BHK-21-like morphology (FIG. 10A) with doubling times ranging from 18 to 30 hours (Table 4). Finally, every single cell was stained positive when immunostaining was performed on 12 stable clones with an anti-double stranded RNA (dsRNA) antibody (FIG. 10B). Because dsRNA is an intermediate product during the replication of positive sense genome viruses, this finding confirmed the existence of active SARS-CoV-2 replication in these clones.

TABLE 4
Doubling time of each clone.

| Name of Clone | Doubling Time (hours) |
|---|---|
| BHK-21-NP^{Dox-ON} | 23.5 |
| Clone 2 | 23.5 |
| Clone 3 | 25 |
| Clone 4 | 21 |
| Clone 5 | 21.8 |
| Clone 6 | 19.9 |
| Clone 7 | 18.3 |
| Clone 8 | 25.8 |
| Clone 9 | 19.4 |
| Clone 10 | 23.9 |
| Clone 11 | 21 |
| Clone 12 | 30.6 |
| Clone 13 | 22.6 |

### Example 8

### Identification of Anti-Viral Compounds Using BHK-21-NP^{Dox-ON} Cells Expressing SARS-CoV-2-Rep-NanoLuc-Neo-Nsp1^{K164A/H165A}

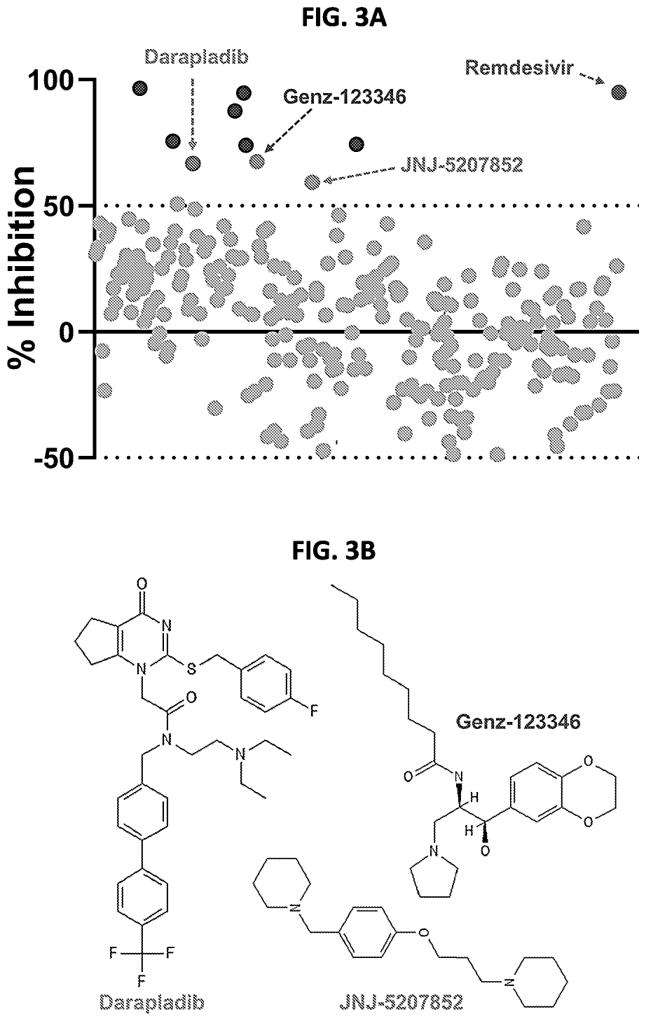

To demonstrate the suitability of SARS-CoV-2-Rep-NanoLuc-Neo-Nsp1^{K164A/H165}A cells in drug screen, a library of 273 compounds was tested for inhibitory effects (Table 5). These compounds were selected to target Nsp5 (3CLpro), Nsp3 (PLpro), Nsp12 (RdRP), Nsp15, Nsp16, and X domain by virtual screen. At 5 μM, nine compounds exhibited more than 50% inhibition based on nanoluciferase expression (FIG. 3A). Three compounds, including Darapladib (predicted to target 3CLpro), Genz-123346 (predicted to target Nsp16), JNJ-5207852 (predicted to target Nsp15) (FIG. 3B), were first validated in replicon cells and then in the three human cell lines A549-hACE2, Calu-3, and Caco-2, using live virus. Remdesivir (inhibitor of RdRP) and GC376 (3CL protease inhibitor) were included as positive controls.

TABLE 5
A library of 273 compounds tested for inhibitory effects on SARS-CoV-2.

| ID | Name | Synonyms | CAS | SMILES | Formula |
|---|---|---|---|---|---|
| T2934 | Bilirubin | Bilibubin; | 635-65-4 | Cc1c([nH]c(c1CCC(═O) | C33H36N4O6 |
| | | Hematoidin; | | O)Cc1c(c(c([nH]1)/ | |
| | | Hemetoidin; | | C═C\1/C(═C(C(═O)N1) | |
| | | Principal | | C)C═C)C)CCC(═O)O)/ | |
| | | bile pigment | | C═C\1/C(═C(C(═O) | |
| | | | | N1)C═C)C | |
| T3281 | Delapril | Delapril | 83435- | CCOC(═O)[C@H] | C26H33ClN2O5 |
| | Hydrochloride | Hydrochloride; | 67-0 | (CCc1ccccc1)N[C@@H] | |
| | | Alindapril | | (C)C(═O)N(CC(═O) | |
| | | Hydrochloride; | | O)C1Cc2ccccc2C1•Cl | |
| | | CV 3317; | | | |
| | | indalapril | | | |
| T5014 | Prostaglandin | Dinoprostone; | 363-24-6 | CCCCC[C@H](O)\C═C\ | C20H32O5 |
| | E2 (PGE2) | Prostaglandin | | [C@H]1[C@H](O) | |
| | | E2; PGE2 | | CC(═O)[C@@H]1C\ | |
| | | | | C═C/CCCC(O)═O | |
| T6109 | Darapladib | SB-480848 | 356057- | CCN(CC)CCN(Cc1ccc | C36H38F4N4O2S |
| | | | 34-6 | (cc1)c1ccc(cc1)C(F) | |
| | | | | (F)F)C(═O)Cn1c2c | |
| | | | | (CCC2)c(═O)nc1SCc1ccc | |
| | | | | (cc1)F | |
| T0158 | Mitoxantrone | Mitoxantrone | 70476- | Cl•OCCNCCNc1c2C(═O) | C22H30Cl2N4O6 |
| | hydrochloride | dihydrochloride; | 82-3 | c3c(O)ccc(O)c3C(═O) | |
| | | mitozantrone | | c2c(NCCNCCO)cc1•Cl | |
| | | dihydrochloride; | | | |
| | | Mitoxantrone | | | |
| | | 2HCl; NSC- | | | |
| | | 301739 | | | |
| G211- | G211-0291 | | 959508- | C(C(═C(C(NCC(═CC1) | C25H25N3O2 |
| 0291 | | | 41-9 | C═CC═1)═O)C | |
| | | | | (C1═CC═C2)═C2)C2)(C | |
| | | | | (N2C(CC2)CCC2)═O)═N1 | |
| T0467 | Sildenafil | UK-92480 | 171599- | OC(═O)CC(O)(CC(O)═O) | C28H38N6O11S |
| | citrate | citrate | 83-0 | C(O)═O•CCCc1nn(C) | |
| | | | | c2c1nc([nH]c2═O)—c1cc | |
| | | | | (ccc1OCC)S(═O) | |
| | | | | (═O)N1CCN(C)CC1 | |
| C924- | C924-0274 | | 890825- | O═C2N(CC(═O)N/C1═C/ | C25H28FN3O2 |
| 0274 | | | 65-7 | C—CC(C)═C1C)C4 | |
| | | | | (/N═C2/C3═C/C═C | |
| | | | | (/[F])C═C3)CCCCCC4 | |
| C519- | C519-1772 | | 902574- | O═[S](═O)(C═2C═C1C | C29H44N4O4S |
| 1772 | | | 29-2 | (═O)C(═CN(CC)C1═CC═2) | |
| | | | | C(═O)NCCCN3C[C@] | |
| | | | | (C)([H])C[C@](C) | |
| | | | | ([H])C3)N(C)[C@]4 | |
| | | | | ([H])CCCCC4 | |
| C697- | C697-0280 | | 902563- | S(C(═C(O1)C═CN(C) | C22H34N4O4S |
| 0280 | | | 73-9 | C)C(═N1)C)(N(CC1C | |
| | | | | (═O)NCCC(═CC2)CCC2) | |
| | | | | CCC1)(═O)═O | |
| T2873 | Ginsenoside | Panaxoside Rg2; | 52286- | C[C@@H]1O[C@@H] | C42H72O13 |
| | Rg2 | Prosapogenin C2; | 74-5 | (O[C@@H]2[C@@H] | |
| | | Chikusetsusaponin I; | | (O)[C@H](O)[C@@H] | |
| | | (20S)Ginsenoside | | (CO)O[C@H]2O | |
| | | Rg2 | | [C@H]2C[C@]3(C) | |
| | | | | [C@H](C[C@@H](O) | |
| | | | | [C@@H]4[C@H](CC | |
| | | | | [C@@]34C)[C@@] | |
| | | | | (C)(O)CC\C═C(/C)C) | |
| | | | | [C@@]3(C)CC[C@H] | |
| | | | | (O)C(C)(C)[C@H]23) | |
| | | | | [C@H](O)[C@H](O) | |
| | | | | [C@H]1O | |
| T2310 | CHIR99021 | CHIR | 252917- | Cc1cnc([nH]1)—c1cnc | C22H18Cl2N8 |
| | | 99021; CHIR- | 06-9 | (NCCNc2ccc(cn2)C#N) | |
| | | 99021; CT99021 | | nc1—c1ccc(Cl)cc1Cl | |
| T5S1103 | Isoliensinine | Isoliensinin | 6817-41- | COc1ccc(C[C@H]2N | C37H42N2O6 |
| | | | 0 | (C)CCc3cc(OC)c(Oc4cc | |
| | | | | (C[C@H]5N(C)CCc6cc | |
| | | | | (OC)c(O)cc56)ccc4O) | |
| | | | | cc23)cc1 | |
| C738- | C738-0291 | | 902954- | C(═C1NCCN(CC(═CC2) | C22H29N3O4 |
| 0291 | | | 26-1 | C═CC═2)C)(C(C1═O)═O) | |
| | | | | N(CC1C(═O)OCC)CCC1 | |
| D072- | D072-0267 | | 855714- | O═[S](═O)(C═1N═C | C20H19FN2O4S |
| 0267 | | | 75-9 | (OC═1NC[C@@]2([H]) | |
| | | | | OCCC2)C═3C═CC | |
| | | | | ([F])═CC═3)C═4C═CC═CC═4 | |
| 2995- | 2995-0491 | | 312598- | N(C(N1)═O)(C(C1(C) | C21H25N3O5S |
| 0491 | | | 19-9 | C)═O)CC(CN(S(C(═CC1) | |
| | | | | C═CC═1C)(═O)═O) | |
| | | | | C(C═C1)═CC═C1)O | |
| T6332 | Pevonedistat | MLN4924 | 905579- | NS(═O)(═O)OC[C@@H] | C21H25N5O4S |
| | | | 51-3 | 1C[C@H](C[C@@H] | |
| | | | | 1O)n1ccc2c(N[C@H] | |
| | | | | 3CCc4ccccc34)ncnc12 | |
| C115- | C115-0510 | | 685868- | O═C3N(C)[C@]([H]) | C27H32N2O7 |
| 0510 | | | 67-1 | (C1═CC═C(C═C1)OC) | |
| | | | | [C@]([H])(C2═CC | |
| | | | | (OC)═C(C═C23)OC)C | |
| | | | | (═O)N4CC[C@@]5(CC4) | |
| | | | | OCCO5 | |
| T4961 | 9′-Methyl | | 1167424- | COC(═O)[C@@H] | C37H32O16 |
| | lithospermate | | 31-8 | (Cc1ccc(O)c(O)c1)OC | |
| | B | | | (═O)[C@H]1[C@@H] | |
| | | | | (Oc2c1c(\C═C\C(═O)O | |
| | | | | [C@H](Cc1ccc(O)c(O) | |
| | | | | c1)C(O)═O)ccc2O) | |
| | | | | c1ccc(O)c(O)c1 | |
| T0772 | Troxerutin | Trihydroxyethyl | 7085-55- | C[C@@H]1O[C@@H] | C33H42O19 |
| | | rutin | 4 | (OC[C@H]2O[C@@H] | |
| | | | | (Oc3c(oc4cc(OCCO) | |
| | | | | cc(O)c4c3═O)—c3ccc | |
| | | | | (OCCO)c(OCCO)c3) | |
| | | | | [C@H](O)[C@@H] | |
| | | | | (O)[C@@H]2O)[C@H] | |
| | | | | (O)[C@H](O)[C@H]1O | |
| T7740 | Protease- | | T7740 | OC(═O)C(F)(F)F•OC(═O) | C37H48F6N8O11 |
| | Activated | | | C(F)(F)F•NCCCC[C@H] | |
| | Receptor-4 | | | (NC(═O)CNC(═O) | |
| | diTFA(245443- | | | [C@@H]1CCCN1C(═O) | |
| | 52-1(free | | | [C@H](CC1═CC═C | |
| | base)) | | | (O)C═C1)NC(═O)CN) | |
| | | | | C(═O)N[C@@H] | |
| | | | | (CC1═CC═CC═C1) | |
| | | | | C(N)═O | |
| T3054 | Daurisoline | (R,R)- | 70553- | CN1CCc2cc(c(cc2[C@H] | C37H42N2O6 |
| | | Daurisoline | 76-3 | 1Cc1ccc(cc1)Oc1c | |
| | | | | (ccc(c1)C[C@@H] | |
| | | | | 1c2cc(c(cc2CCN1C) | |
| | | | | OC)O)O)OC)OC | |
| T2S0257 | Dehydroandro- | | 786593- | C[C@@]1(COC(═O) | C28H36O10 |
| | grapholide | | 06-4 | CCC(O)═O)[C@@H] | |
| | succinate | | | (CC[C@]2(C)[C@@H] | |
| | | | | 1CCC(═C)[C@H]2\ | |
| | | | | C═C\C1═CCOC1═O) | |
| | | | | OC(═O)CCC(O)═O | |
| T5726 | Specneuzhenide | Nuezhenide | 449733- | COC(═O)C1═CO[C@@H] | C31H42O17 |
| | | | 84-0 | (O[C@@H]2O[C@H] | |
| | | | | (CO)[C@@H](O) | |
| | | | | [C@H](O)[C@H]2O)\ | |
| | | | | C(═C/C)[C@@H]1CC | |
| | | | | (═O)OC[C@H]1O[C@@H] | |
| | | | | (OCCc2ccc(O)cc2) | |
| | | | | [C@H](O)[C@@H] | |
| | | | | (O)[C@@H]1O | |
| T1795 | Carfilzomib | PR-171 | 868540- | O1[c@@](C(═O)[C@@H] | C40H57N5O7 |
| | | | 17-4 | (NC(═O)[C@@H] | |
| | | | | (NC(═O)[C@@H] | |
| | | | | (NC(═O)[C@@H](NC | |
| | | | | (═O)CN2CCOCC2) | |
| | | | | CCc2ccccc2)CC(C)C) | |
| | | | | Cc2ccccc2)CC(C)C) | |
| | | | | (C1)C | |
| T4049 | Genz-123346 | | 491833- | C(═O)(CCCCCCCC) | C24H38N2O4 |
| | free base | | 30-8 | N[C@@H]([C@H](O) | |
| | | | | c1cc2c(OCCO2)cc1) | |
| | | | | CN1CCCC1 | |
| T6130 | Skepinone-L | CBS3830 | 1221485- | OC[C@@H](O) | C24H21F2NO4 |
| | | | 83-1 | COc1ccc2CCc3cc(Nc4ccc(F) | |
| | | | | cc4F)ccc3C(═O)c2c1 | |
| T6917 | Oleuropein | | 32619- | COC(═O)C1═CO[C@@H] | C25H32O13 |
| | | | 42-4 | (O[C@@H]2O[C@H] | |
| | | | | (CO)[C@@H](O) | |
| | | | | [C@H](O)[C@H]2O)\ | |
| | | | | C(═C\C)[C@@H]1CC | |
| | | | | (═O)OCCc1ccc(O)c(O)c1 | |
| T3099 | Pinometostat | EPZ-5676 | 1380288- | CC(C)N(C[C@H]1O[ | C30H42N8O3 |
| | | | 87-8 | C@H]([C@H](O) | |
| | | | | [C@@H]1O)n1cnc2c(N) | |
| | | | | ncnc12)[C@@H]1C | |
| | | | | [C@H](CCc2nc3ccc | |
| | | | | (cc3[nH]2)C(C)(C)C)C1 | |
| T0447 | Carvedilol | BM 14190; SKF | 72956- | COc1ccccc1OCCNCC(O) | C24H26N2O4 |
| | | 105517 | 09-3 | COc1cccc2c1c1ccccc1 | |
| | | | | [nH]2 | |
| T5841 | Travoprost | Fluprostenol | 157283- | CC(C)OC(═O)CCC\C═C/ | C26H35F3O6 |
| | | isopropyl | 68-6 | CC1C(O)CC(O)C1\ | |
| | | ester; AL6221; | | C═C\C(O)COc1cccc | |
| | | Flu-Ipr | | (c1)C(F)(F)F | |
| T5234 | Glycoursode- | Ursodeoxy- | 64480- | C[C@H](CCC(═O)NCC | C26H43NO5 |
| | oxycholic | cholylglycine; GUDCA | 66-6 | (O)═O)[C@H]1CC[C@H] | |
| | acid | | | 2[C@@H]3[C@@H] | |
| | | | | (O)C[C@@H] | |
| | | | | 4C[C@H](O)CC[C@] | |
| | | | | 4(C)[C@H]3CC[C@] | |
| | | | | 12C | |
| T4678 | Fmoc-Val-Cit- | | 159858- | CC(C)[C@H](NC(═O) | C33H39N5O6 |
| | PAB | | 22-7 | OCC1c2ccccc2—c2ccccc12) | |
| | | | | C(═O)N[C@@H] | |
| | | | | (CCCNC(N)═O)C(═O) | |
| | | | | Nc1ccc(CO)cc1 | |
| C522- | C522-0732 | | 901716- | N(C(═C1C)CN(CC2) | C24H31ClN4O3 |
| 0732 | | | 39-0 | CCC2C(═O)NCCCN | |
| | | | | (C(═O)C2)CC2)═C(O1) | |
| | | | | C(C═C1)═CC═C1[Cl] | |
| E859- | E859-1859 | | 894561- | C(C(═C(N1C(C═C2)═CC═C2) | C28H32N6O2 |
| 1859 | | | 23-0 | C)C2C)(C(═NN═2) | |
| | | | | N(CC2C(NCC(═CC3) | |
| | | | | C═CC═3OCC)═O)CCC2)═N1 | |
| T3579 | PLX8394 | | 1393466- | F[C@@H]1CCN(C1) | C25H21F3N6O3S |
| | | | 87-9 | S(═O)(═O)Nc1ccc(F)c | |
| | | | | (C(═O)c2c[nH]c3ncc | |
| | | | | (cc23)—c2cnc(nc2)C2CC2)c1F | |
| D011- | D011-0999 | | 883644- | C(═N1)(N(C(C1═CC1)═CC═1) | C29H31N3O4 |
| 0999 | | | 08-4 | CCCOC(═CC1)C═CC═1OC) | |
| | | | | C1CN(C(C1)═O)C(C | |
| | | | | (OCC)═C1)═CC═C1 | |
| D126- | D126-0074 | | 901663- | C(═C(C1C(C(═C2) | C27H33N3O5 |
| 0074 | | | 71-6 | O)═CC(═C2)C)C2C | |
| | | | | (═CC(═C(C3)OCCC(C)C) | |
| | | | | OC)C═3)(C(N2CCCO)═O)NN═1 | |
| C147- | C147-0154 | | 825601- | C/C1═C/C═C/C═C1/NC/C3═N/ | C24H25N3O |
| 0154 | | | 75-0 | C═2C═CC═CC═2N3CCO/ | |
| | | | | C4═C/C═C(/C)C═C4 | |
| T2544 | Bazedoxifene | WAY-TES | 198481- | C(═O)(C)O•c1c(cc2c | C32H38N2O5 |
| | acetate | 424; TSE | 33-3 | (c1)n(c(c2C)c1ccc(cc1)O) | |
| | | 424; WAY- | | Cc1ccc(cc1)OCCN1CCCCCC1)O | |
| | | 140424 | | | |
| 8015- | 8015-6465 | | 696634- | O═C2N(CCCC(═O)C1═C | C22H26N2O5 |
| 6465 | | | 21-6 | (CC(C)(C)CC1═O)NCCO)C | |
| | | | | (═O)C═3C═CC═CC2═3 | |
| T6162 | BS-181 HCl | BS-181 | 1397219- | Cl•CC(C)c1cnn2c | C22H32N6•HCl |
| | | hydrochloride | 81-6 | (NCc3ccccc3)cc(NCCCCCCN) | |
| | | | | nc12 | |
| T6883 | LY3023414 | GTPL8918 | 1386874- | O═c1n(c2c(c3cc(c4cc | C23H26N4O3 |
| | | | 06-1 | (C(O)(C)C)cnc4)ccc3nc2) | |
| | | | | n1C[C@H](C)OC)C | |
| T6S1768 | Narcissoside | Narcissin; | 604-80-8 | COc1cc(ccc1O)—c1oc2cc | C28H32O16 |
| | | Isorhamnetin 3- | | (O)cc(O)c2c(═O)c1O[C@@H] | |
| | | Rutinoside | | 1O[C@H](CO[C@@H]2O | |
| | | | | [C@@H](C)[C@H](O) | |
| | | | | [C@@H](O)[C@H]2O) | |
| | | | | [C@@H](O)[C@H](O)[C@H]1O | |
| T3217 | PF-CBP1 | PF-CBP1 HCl | 2070014- | CCCOc1ccc(cc1)CCc1nc2c | C29H37ClN4O3 |
| | hydrochloride | | 93-4 | (n1CCN1CCOCC1)ccc(c2) | |
| | | | | c1c(onc1C)C•Cl | |
| E589- | E589-2554 | | 892739- | S(C(C═C(N(C1═O)CC(NC | C22H25N3O6S2 |
| 2554 | | | 01-4 | (C═C2OC)═C(C═C2)OC)═O) | |
| | | | | C(SC1)═C1)═C1)(N(CC1)CC1) | |
| | | | | (═O)═O | |
| G071- | G071-0431 | | 895101- | S(N(C1═CC2)CC(C | C28H26N4O3S2 |
| 0431 | | | 33-4 | (C)═C3)═CC═C3)(C(═C | |
| | | | | (C1═CC═2)N1)C═NC═1SCC | |
| | | | | (═O)NCCC(═CC1) | |
| | | | | C═CC═1)(═O)═O | |
| 7472- | 7472-0051 | | 892246- | C(N(CC1C)CC(O1)C) | C22H23FN4O2S |
| 0051 | | | 53-6 | (═NC(═C1C═C2)C═C2) | |
| | | | | C(═N1)SCC(NC | |
| | | | | (C═C1)═CC═C1F)═O | |
| D074- | D074-0222 | | 879938- | O═C2C═1NN═C(C═1 | C30H31N3O4 |
| 0222 | | | 29-1 | [C@@]([H]) | |
| | | | | (N2CCCOC(C)(C)[H])C═4/ | |
| | | | | C═C(/OCC═3C═CC═CC═3) | |
| | | | | C═CC═4)C5═CC═C/ | |
| | | | | C═C5/O | |
| T5318 | CREB | CREB inhibitor | 1433286- | Cl•NCCCOc1cc2ccccc2cc1C | C33H31C12N3O5 |
| | inhibitor | | 70-4 | (═O)NCCOc1cc2ccccc2cc1C | |
| | | | | (═O)Nc1ccc(Cl)cc1O | |
| T3108 | CUDC101 | CUDC | 1012054- | n1cnc(c2c1cc(c(c2) | C24H26N4O4 |
| | | 101; CUDC-101 | 59-9 | OCCCCCCC(═O)NO)OC) | |
| | | | | Nc1cc(ccc1)C#C | |
| T2727 | Salvianolic | | 115939- | OC(═O)[C@@H](Cc1ccc | C36H30O16 |
| | acid B | | 25-8 | (O)c(O)c1)OC(═O)\ | |
| | | | | C═C\c1ccc(O)c2O[C@@H] | |
| | | | | ([C@@H](C(═O) | |
| | | | | O[C@H](Cc3ccc(O)c(O) | |
| | | | | c3)C(O)═O)c12)c1ccc(O)c(O)c1 | |
| G114- | G114-0456 | | 895099- | S(C(C═C(N(C1═O)CC | C20H23N3O4S2 |
| 0456 | | | 65-7 | (NC(═CC2)C═CC═2CC)═O) | |
| | | | | C(SC1)═C1)═C1) | |
| | | | | (N(C)C)(═O)═O | |
| K788- | K788-6101 | | 899212- | O═C3N(CC(═O)NCCCN1CCN | C25H31ClFN5O2 |
| 6101 | | | 06-7 | (CC1)C2═C/C═C(/[F]) | |
| | | | | C═C2)CCN3C/C4═C/C═C | |
| | | | | (/[Cl])C═C4 | |
| G240- | G240-0046 | | 895252- | S(NC(═CC1OC)C═C | C21H24N2O7S |
| 0046 | | | 40-1 | (C═1)OC)(C(C═C(C | |
| | | | | (OC1C(N(CC2)CC2)═O)═C2) | |
| | | | | OC1)═C2)(═O)═O | |
| C200- | C200-4180 | | 892305- | N(C(═N1)SCC(NC | C24H24ClN5O4S |
| 4180 | | | 29-2 | (C═C2OC)═C(C═C2)OC)═O) | |
| | | | | (C(C(C1═C1)═NN1CC)═O) | |
| | | | | CC(C═C1)═CC═C1[Cl] | |
| K784- | K784-0203 | | 422555- | S(C(═CC(C(NC(═C(C1)C) | C23H27FN2O3S |
| 0203 | | | 16-6 | C═C(C═1)C)═O)═C | |
| | | | | (C1)F)C═1)(═O)(═O) | |
| | | | | NCCC(═CC1)CCC1 | |
| G406- | G406-0489 | | 959561- | S(N(CC1)CCC1)(C1═CC | C24H29N3O6S |
| 0489 | | | 08-1 | (═C(N(C2═O)CC(NC | |
| | | | | (C═C3OC)═C(C═C3) | |
| | | | | OC)═O)C═C1)CC2)(═O)═O | |
| C336- | C336-0153 | | 959510- | O═C1N4C(═N[C@@] | C28H31N5O4S |
| 0153 | | | 20-4 | 1([H])CC(═O)N[C@@] | |
| | | | | 2([H])CCCCC2) | |
| | | | | C═3C═CC═CC═3/N═C4/ | |
| | | | | [S]CC(═O)N/C5═C/C═C/ | |
| | | | | C═C5/OCC | |
| K786- | K786-5309 | | 697278- | O═C1C[C@]([H]) | C20H32N2O2 |
| 5309 | | | 01-6 | (CN1[C@@]2([H])CCCCCC2) | |
| | | | | C(═O)NCC/C3═C/CCCC3 | |
| T3952 | TPEN | TPEDA | 16858- | C(CN(Cc1ccccn1) | C26H28N6 |
| | | | 02-9 | Cc1ccccn1)N(Cc1ccccn1) | |
| | | | | Cc1ccccn1 | |
| Y050- | Y050-2147 | | 887600- | O═[S](═O)(N1CCOCC1) | C18H26N2O6S |
| 2147 | | | 07-9 | C3═CC(C)═C(OCC(═O) | |
| | | | | NC[C@]2([H])OCCC2)C═C3 | |
| T3112 | Verteporfin | CL | 129497- | COC(═O)CCc1c(C)c2cc3 | C41H42N4O8 |
| | | 318952; BPD- | 78-5 | [nH]c(cc4nc(cc5[nH] | |
| | | MA | | c(cc1n2)c(CCC(O)═O)c5C)c | |
| | | | | (C═C)c4C)C1═CC═C | |
| | | | | ([C@@H](C(═O)OC) | |
| | | | | [C@@]31C)C(═O) | |
| | | | | OC•COC(═O)CCc1c(C) | |
| | | | | c2cc3nc(cc4[nH]c(cc5nc | |
| | | | | (cc1[nH]2)c(CCC(O)═O) | |
| | | | | c5C)[C@]1(C)[C@H] | |
| | | | | (C(═O)OC)C(═CC═C41) | |
| | | | | C(═O)OC)c(C)c3C═C | |
| E946- | E946-0756 | | 950395- | C(C(═C1N2)C(═NC═2C) | C24H31N5O3 |
| 0756 | | | 17-2 | NCCCN(CC2)CCN2C(═CC2) | |
| | | | | C═CC═2)(═C(O1) | |
| | | | | C)C(═O)OCC | |
| K784- | K784-7502 | | 688796- | O═C(NC═1/C═C(/[Cl]) | C22H20ClN5OS |
| 7502 | | | 67-0 | C═CC═1)C[S]C═4N═C3/ | |
| | | | | N═C(/C)C | |
| | | | | (CC═2C═CC═CC═2)═C(C)N3N═4 | |
| E642- | E642-1065 | | 893161- | N(C(═N1)SCC(C═C2F)═CC═C2) | C29H25FN4OS |
| 1065 | | | 16-5 | (C(C1═C1)═NC═C1) | |
| | | | | CC(═CC1)C═CC═1C | |
| | | | | (═O)NCCC(═CC1)C═CC═1 | |
| T4259 | GLPG0187 | | 1320346- | COc1ccc(cc1)S(═O)(═O) | C29H37N7O5S |
| | | | 97-1 | N[C@@H](CNc2nc(C) | |
| | | | | nc(c2C)N3CCC(CC3) | |
| | | | | c4ccc5CCCNc5n4)C(═O)O | |
| T3631 | PF8380 | PF 8380; PF- | 1144035- | C1CN(CCN1CCC(═O) | C22H21Cl2N3O5 |
| | | 8380 | 53-9 | c1cc2c(cc1)[nH]c(═O) | |
| | | | | o2)C(═O)OCc1cc(cc(c1)Cl)Cl | |
| G211- | G211-0145 | | 959490- | O═C3C2═NC1═CC═CC═C1C | C26H27N3O3 |
| 0145 | | | 59-6 | (═C2CN3C4([H])CCCCC4) | |
| | | | | C(═O)NCC═5C═CC(═CC═5)OC | |
| K279- | K279-1256 | | 958963- | N(C(═N1)C(═C2C═C3) | C23H22N4O4S |
| 1256 | | | 23-0 | C═C3)(C(═N2)SCC | |
| | | | | (═O)OCC)C(C1CC(NCC | |
| | | | | (═CC1)C═CC═1)═O)═O | |
| K405- | K405-3134 | | 890889- | N(C(═C1C2NCCC(═CC3) | C21H25N5 |
| 3134 | | | 69-7 | CCC3)N═CN═2) | |
| | | | | (N═C1)C(═CC(═C(C1) | |
| | | | | C)C)C═1 | |
| E216- | E216-4969 | | 891882- | O═C(NC═1C═CC═CC═1C | C20H22N2O4 |
| 4969 | | | 89-6 | (═O)OCC)N2C[C@] | |
| | | | | (C)([H])OC3═C/C═C | |
| | | | | (/C)C═C23 | |
| C547- | C547-0142 | | 901729- | C(C1)(N(C(C(C═1OCC | C22H24N2O4 |
| 0142 | | | 44-0 | (N(C(═CC1)C═CC═1OCC) | |
| | | | | CC)═O)═C1)═CC═C1) | |
| | | | | C)═O | |
| C880- | C880-2612 | | 906252- | O═C1C4═C(C═NN1CC | C28H25ClN4O2 |
| 2612 | | | 12-8 | (═O)NCCC/C2═C/ | |
| | | | | C═CC═C2)C3═CC═CC═C3N4C/ | |
| | | | | C5═C/C═C(/[Cl])C═C5 | |
| E542- | E542-1696 | | 892370- | O═C(NCC/C1═C/C═C | C29H32N2O2 |
| 1696 | | | 71-7 | (C═C1)OCC)CC/C4═C/ | |
| | | | | N(CC═2C═CC(C)═CC═2) | |
| | | | | C3═CC═CC═C34 | |
| T7210 | Guanosine 5′- | GDP | 146-91-8 | Nc1nc2n(cnc2c(═O)[nH]1) | C10H15N5O11P2 |
| | diphosphate | | | [C@@H]1O[C@H] | |
| | | | | (COP(O)(═O)OP(O)(O)═O) | |
| | | | | [C@@H](O)[C@H]1O | |
| T5330 | Fluralaner | AH252723; | 864731- | Cc1cc(ccc1C(═O)NCC | C22H17Cl2F6N3O3 |
| | | A1443 | 61-3 | (═O)NCC(F)(F)F)C1═NOC | |
| | | | | (C1)(c1cc(Cl)cc(Cl) | |
| | | | | c1)C(F)(F)F | |
| 8009- | 8009-8507 | | 309283- | N(C(═C1C2)C═C(C(F) | C25H16F3NOS |
| 8507 | | | 33-8 | (F)F)C═2)(CC(C(C(═C2C3) | |
| | | | | C═CC═3)═CC═C2)═O) | |
| | | | | C(═C(S1)C1)C═CC═1 | |
| T7509 | PD 117519 | CI947 | 96392- | OCC1OC(n2cnc3c | C19H21N5O4 |
| | | | 15-3 | (NC4CCc5ccccc54)ncnc32) | |
| | | | | C(O)C1O | |
| TMO2681 | Xanthosine | Xanthine | 146-80-5 | OC[C@H]1O[C@H] | C10H12N4O6 |
| | | riboside; 9-Beta- | | ([C@H](O)[C@@H]1O) | |
| | | D- | | n1cnc2c1[nH]c(═O) | |
| | | Ribofuranosylxanthine | | [nH]c2═O | |
| 6623- | 6623-1226 | | 350497- | O═C(N/C1═C/C(═CC═C1) | C22H21N5O2S |
| 1226 | | | 73-3 | C(═O)C)C[S]/C2═N/ | |
| | | | | C4═C(N═N2) | |
| | | | | C═3C═CC═CC═3N4CCC | |
| T0148L | Folinic Acid | Leucovorin | 6035-45- | C1C(N(c2c(N1)[nH]c(nc2═O) | C20H21CaN7O7•5H2O |
| | Calcium Salt | calcium salt | 6 | N)C═O)CNc1ccc(cc1) | |
| | Pentahydrate | pentahydrate; | | C(═O)N[C@@H] | |
| | | Leucovorin | | (CCC(═O)[O—])C(═O) | |
| | | Calcium | | [O—]•O•[Ca + | |
| | | Pentahydrate; | | 2]•O•O•O•O | |
| | | Folinic acid | | | |
| T6676 | Sofosbuvir | GS 7977; PSI- | 1190307- | [P@@](═O)(OC[C@H] | C22H29FN3O9P |
| | | 7977 | 88-0 | 1O[C@H]([C@](F) | |
| | | | | ([C@@H]1O)C)n1ccc | |
| | | | | (═O)[nH]c1═O)(Oc1ccccc1) | |
| | | | | N[C@H](C(═O)OC(C)C)C | |
| T4060 | Acelarin | NUC-1031 | 840506- | C[C@H](NP(═O) | C25H27F2N4O8P |
| | | | 29-8 | (Oc1ccccc1)OC[C@H]1O | |
| | | | | [C@@H](n2ccc(N)nc2═O) | |
| | | | | C(F)(F)[C@@H]1O)C | |
| | | | | (═O)OCc1ccccc1 | |
| C241- | C241-1670 | | 912800- | N(C(N1CC(NC(═CC2 | C28H27ClN4O6S |
| 1670 | | | 78-3 | [Cl])C═CC═2)═O)═O) | |
| | | | | (C(C(═C1C1)SC═1)═O) | |
| | | | | CCCCCC(NCC1═CC | |
| | | | | (═C(O2)C═C1)OC2)═O | |
| T3893 | Forsythoside B | | 81525- | O1[C@H]([C@@H] | C34H44O19 |
| | | | 13-5 | ([C@H]([C@@H][C@H] | |
| | | | | 1CO[C@@H]1OC[C@] | |
| | | | | ([C@H]1O)(O)CO) | |
| | | | | OC(═O)/C═C/c1cc(c(cc1)O)O)O) | |
| | | | | [C@@H]1O[C@H]([C@@H] | |
| | | | | ([C@H]([C@H]1O)O) | |
| | | | | O)C)O)OCCc1cc(c(cc1)O)O | |
| T7086 | TBTA | | 510758- | C(N(Cc1cn(Cc2ccccc2) | C30H30N10 |
| | | | 28-8 | nn1)Cc1cn(Cc2ccccc2) | |
| | | | | nn1)c1cn(Cc2ccccc2)nn1 | |
| T3780 | Oroxin B | Hypocretin-2 | 114482- | OC[C@H]1O[C@@H] | C27H30O15 |
| | | | 86-9 | (OC[C@H]2O[C@@H] | |
| | | | | (Oc3cc4oc(cc(═O)c4c(O) | |
| | | | | c3O)—c3ccccc3)[C@H](O) | |
| | | | | [C@@H](O)[C@@H] | |
| | | | | 2O)[C@H](O)[C@@H] | |
| | | | | (O)[C@@H]1O | |
| C700- | C700-0693 | | 902482- | O═[S](═O)(C═3C═C2N | C24H36N4O6S |
| 0693 | | | 58-0 | (CC(═O)NCCCN1CCCC | |
| | | | | [C@@]1([H])CC)C | |
| | | | | (═O)COC2═CC═3)N4CCOCC4 | |
| C880- | C880-0271 | | 906237- | C(═C(C1═CC2)C3)(N | C28H34N6O2 |
| 0271 | | | 73-8 | (C1═CC═2)C)C(N(N═3) | |
| | | | | CC(═O)NCCCN(CC1) | |
| | | | | CCN1C(C(═C1)C)═CC | |
| | | | | (═C1)C)═O | |
| T5345 | V9302 | V 9302; V-9302 | 1855871- | Cc1cccc(COc2ccccc2CN | C34H38N2O4 |
| | | | 76-9 | (CC[C@H](N)C(O)═O) | |
| | | | | Cc2ccccc2OCc2cccc(C)c2)c1 | |
| T0672 | Pravastatin | CS-514 | 81131- | [C@H]12[C@H](C | C23H35NaO7 |
| | sodium | (sodium); CS- | 70-6 | [C@H](O)C═C1C═C | |
| | | 514 Sodium | | [C@H](C)[C@@H] | |
| | | | | 2CC[C@@H](O)C[C@@H] | |
| | | | | (O)CC(═O)[O—])OC | |
| | | | | (═O)[C@@H](C) | |
| | | | | CC•[Na+] | |
| T3670 | Forsythoside A | Forsythiaside | 79916- | C[C@@H]1O[C@@H] | C29H36O15 |
| | | | 77-1 | (OC[C@H]2O[C@@H] | |
| | | | | (OCCc3ccc(O)c(O)c3) | |
| | | | | [C@H](O)[C@@H] | |
| | | | | (O)[C@@H]2OC | |
| | | | | (═O)\C═C\c2ccc(O)c(O) | |
| | | | | c2)[C@H](O)[C@H] | |
| | | | | (O)[C@H]1O | |
| T4255 | TM5275 | TM5275 sodium | 1103926- | [Na+]•[O—]C | C28H27ClN3NaO5 |
| | sodium | salt | 82-4 | (═O)c4cc(Cl)ccc4NC | |
| | | | | (═O)COCC(═O)N1CCN | |
| | | | | (CC1)C(c2ccccc2)c3ccccc3 | |
| 6747- | 6747-0106 | | | O═C(C)O[C@@]([H]) | C19H20N2O4 |
| 0106 | | | | (CN1C═NC2═CC═CC═C12) | |
| | | | | CO/C3═C/C═C(C═C3)OC | |
| E946- | E946-0779 | | 950308- | C(C(═C1N2)C(═NC═2C) | C25H32N4O3 |
| 0779 | | | 80-2 | NCC(CC2)CCN2CC | |
| | | | | (═CC2)C═CC═2C) | |
| | | | | (═C(O1)C)C(═O)OCC | |
| D072- | D072-0556 | | 862742- | C(N═C1C(═CC2)C═CC═2) | C25H22ClN3O3S |
| 0556 | | | 56-1 | (═C(O1)N(CC1)CCN1C | |
| | | | | (C═C1[Cl])═CC═C1) | |
| | | | | S(═O)(═O)C(═CC1)C═CC═1 | |
| 6286- | 6286-0223 | | 615280- | C(═N1)(N(C(C1═CC1)═CC═1) | C26H28N2O2 |
| 0223 | | | 85-8 | CCOC(═CC1)C═CC═1) | |
| | | | | COC(═CC1)C═CC═1C(C)(C)C | |
| C448- | C448-1053 | | 901863- | C(═C(C(N(N1)C | C27H33N5O3 |
| 1053 | | | 37-4 | (C═C2)═CC═C2OC)═O) | |
| | | | | N2C)(C═1C(═O)NCCN | |
| | | | | (CC)CCCC)C(C2═C1)═CC═C1 | |
| T5416 | T-5224 | | 530141- | OC(═O)CCc1cc(ccc1OCc1ccc2c | C29H27NO8 |
| | | | 72-1 | (c1)o[nH]c2═O)C(═O) | |
| | | | | c1ccc(OC2CCCC2)cc1O | |
| C636- | C636-2422 | | 1037192- | N(C(N1CCC(═CC2) | C26H28FN3O3 |
| 2422 | | | 29-2 | CCC2)═O)(C(C1CC(NC | |
| | | | | (C═C1)═CC═C1F)═O)═O) | |
| | | | | C(═CC1)C═CC═1C | |
| C336- | C336-0089 | | 1037293- | O═C1N4C(═N[C@]1([H]) | C27H28ClN5O3S |
| 0089 | | | 40-5 | CCC(═O)N[C@]2([H]) | |
| | | | | CCCCC2)C═3C═CC═CC═3/ | |
| | | | | N═C4/[S]CC(═O)N/C5═C/ | |
| | | | | C([Cl])═CC═C5 | |
| T1938 | FLT3-IN-2 | 0 | 923562- | FC(F)(F)c1ccc(CNc2ncc | C21H16ClF3N4 |
| | | | 23-6 | (Cc3c[nH]c4ncc(Cl) | |
| | | | | cc34)cc2)cc1 | |
| T2132 | Buspirone | Buspirone | 33386- | Cl•O═C1CC2(CCCC2) | C21H32ClN5O2 |
| | hydrochloride | HCl; Buspar; Narol | 08-2 | CC(═O)N1CCCCN1CCN(CC1) | |
| | | | | c1ncccn1 | |
| T5847 | Cloprostenol | DL- | 55028- | [Na+]•OC(COc1cccc | C22H28ClNaO6 |
| | sodium | Cloprostenol | 72-3 | (Cl)c1)\C═C\C1C(O)CC | |
| | | sodium; ICI | | (O)C1C\C═C/CCCC | |
| | | 80996 sodium | | ([O—])=O | |
| | | salt; Cloprostenol | | | |
| | | sodium salt | | | |
| T3263 | Cefminox | Meicelin; | 92636- | [Na+]•CO[C@]1(NC | C16H19N7Na2O7S3 |
| | Sodium | Alteporina; | 39-0 | (═O)CSC[C@@H](N) | |
| | | Tencef | | C(O)═O)[C@H]2SCC | |
| | | | | (CSc3nnnn3C)═C(N2C1═O) | |
| | | | | C([O—])═O | |
| 8539- | 8539-0868 | | 950264- | C(N(N1)C2)(N═C(C(C | C25H22ClN3O4 |
| 0868 | | | 69-4 | (═C(OCC(═O)OCC)C3) | |
| | | | | C═C(C═3)CC)═O) | |
| | | | | C═2)═CC═1C(═CC1) | |
| | | | | C═CC═1[C1] | |
| T5384 | RS 504393 | | 300816- | Cc1oc(nc1CCN1CCC2(CC1) | C25H27N3O3 |
| | | | 15-3 | OC(═O)Nc1ccc(C) | |
| | | | | cc21)—c1ccccc1 | |
| K935- | K935-0047 | | | O═C(N[C@]([H]) | C30H29ClN2O4 |
| 0047 | | | | (C/C1═C/NC═2C═CC═CC1═2) | |
| | | | | C(═O)OCC(═O)C═3C═CC | |
| | | | | ([Cl])═CC═3)C4═CC═C | |
| | | | | (C═C4)C(C)(C)C | |
| 8014- | 8014-1195 | | 1008072- | N(N(N1)CC(NC(C(═O)OCC) | C20H21N5O3 |
| 1195 | | | 65-8 | CC(═CC2)C═CC═2)═O)═C | |
| | | | | (N═1)C(═CC1)C═CC═1 | |
| T2375 | BX471 | BX 471; BX- | 217645- | [C@@H]1(N(C(═O) | C21H24ClFN4O3 |
| | | 471; ZK-811752 | 70-0 | COc2ccc(Cl)cc2NC(═O) | |
| | | | | N)CCN(Cc2ccc(F)cc2)C1)C | |
| E843- | E843-0272 | | 894932- | N(C(N1CC(═CC2) | C25H18N4O5 |
| 0272 | | | 72-0 | C═CC═2)═O)(C(C | |
| | | | | (C1═C1)═CC(═C1O1) | |
| | | | | OC1)═O)CC(═NClC(═CC2) | |
| | | | | C═CC═2)ON═1 | |
| G768- | G768-1619 | | 959516- | O═C2C—C(N═C1[S]C | C26H28N6O3S |
| 1619 | | | 20-2 | (═NN12)C3═CC═C | |
| | | | | (C═C3)OC)CN4CCN(CC4) | |
| | | | | C(═O)N/C5═C/C | |
| | | | | (C)═C/C═C5/C | |
| T6179 | Latamoxef | Antibiotic | 64953- | [Na+]•[Na+]•CO[C@] | C20H18N6Na2O9S |
| | sodium | 6059S; LY- | 12-4 | 1(NC(═O)C(C([O—])═O) | |
| | | 127935; Moxalactam | | c2ccc(O)cc2)[C@H]2OCC | |
| | | Disodium; | | (CSc3nnnn3C)═C(N2C1═O)C | |
| | | FestamoxinLy; | | ([O—])═O | |
| | | Shiomarin; 6059 S | | | |
| T5725 | lithospermic | Dan Shen Suan | 121521- | OC(═O)[C@@H](Cc1ccc | C36H30O16 |
| | acid B | B; Salvianolic | 90-2 | (O)c(O)c1)OC(═O)\ | |
| | | acid B | | C═C\c1ccc(O)c2O[C@H] | |
| | | | | ([C@H](C(═O)O | |
| | | | | [C@H](Cc3ccc(O)c(O) | |
| | | | | c3)C(O)═O)c12)c1ccc(O)c(O)c1 | |
| T0136 | Citicoline | CDP- | 987-78-0 | C[N+](C)(C)CCOP(═O)([O—]) | C14H26N4O11P2 |
| | | Choline; cytidine | | OP(═O)(O)OC[C@H]1O[C@@H] | |
| | | 5′- | | (n2ccc(N)nc2═O)[C@H](O) | |
| | | diphosphocholine; | | [C@@H]1O | |
| | | cytidine | | | |
| | | diphosphate- | | | |
| | | choline; Citicholine | | | |
| 8015- | 8015-9350 | | 695204- | C(C(N1)C(C═C(C(O2)═C3) | C23H24ClN3O5 |
| 9350 | | | 55-8 | OC2)═C3)(═C(NC1═O) | |
| | | | | CNCCC(═CC1)C═CC═1[Cl]) | |
| | | | | C(═O)OCC | |
| 8015- | 8015-7947 | | 696653- | C(C(N1)C(C═C(C(O2)═C3) | C24H27N3O7 |
| 7947 | | | 08-4 | OC2)═C3)(═C(NC1═O) | |
| | | | | CNCCOC(C═C1)═CC═C1OC) | |
| | | | | C(═O)OCC | |
| T0154 | Nebivolol | R 065824 | 152520- | Cl•O[C@@H](CNC[C@H] | C22H25F2NO4•HCl |
| | hydrochloride | hydrochloride; | 56-4 | (O)[C@@H]1CCc2cc | |
| | | Nebivolol HCl; R- | | (F)ccc2O1)[C@H]1CCc2cc(F)ccc2O1 | |
| | | 65824 | | | |
| T3409 | Plantamajoside | Y0160; C10485 | 104777- | C1═CC(═C(C═C1CCO[C@H] | C29H36O16 |
| | | | 68-6 | 2[C@@H]([C@H] | |
| | | | | ([C@@H]([C@H] | |
| | | | | (O2)CO)OC(═O)/C═C/C3═CC | |
| | | | | (═C(C═C3)O)O)O[C@H] | |
| | | | | 4[C@@H]([C@H] | |
| | | | | ([C@@H]([C@H](O4)CO) | |
| | | | | O)O)O)O)O)O | |
| C528- | C528-0901 | | 901875- | C(N═C1C(C═C2OC)═CC═C2) | C20H27N3O6S |
| 0901 | | | 64-7 | (CS(CC(═O)NCCN(CC2)CCO2) | |
| | | | | (═O)═O)═C(O1)C | |
| K284- | K284-3774 | | 422285- | O═C2N(CCCC(═O) | C27H24FN3O3S |
| 3774 | | | 64-1 | NCC═1C═CC═CC═1)C | |
| | | | | (═NC3═CC═CC═C23) | |
| | | | | [S]CC(═O)C4═C/C═C | |
| | | | | (/[F])C═C4 | |
| C527- | C527-0061 | | 958954- | O═[S](CC═1N═C(OC═1C) | C22H22N2O6S |
| 0061 | | | 53-5 | C═2C═CC(═CC═2)OC) | |
| | | | | CC(═O)NC/C4═C/ | |
| | | | | C═3OCOC═3C═C4 | |
| G678- | G678-0299 | | 904828- | S(N(CC1)CCN1C(═CC1) | C22H28FN3O5S |
| 0299 | | | 70-2 | C═CC═1F)(CCNC(═O) | |
| | | | | COC(═CC1)C═CC═1OCC) | |
| | | | | (═O)═O | |
| C060- | C060-0100 | | 443331- | O═C1C5═C(N═CN1CC | C26H31N5O5S |
| 0100 | | | 83-7 | (═O)NCCCC(═O)N4CCN | |
| | | | | (CC═3C═C2OCOC2═CC═3) | |
| | | | | CC4)[S]C(C)═C5C | |
| T3708 | BP-1-102 | | 1334493- | CN(CC(═O)N(Cc1ccc | C29H27F5N2O6S |
| | | | 07-0 | (cc1)C1CCCCC1)c1cc | |
| | | | | (c(cc1)C(═O)O)O)S | |
| | | | | (═O)(═O)c1c(c(c(c(c1F) | |
| | | | | F)F)F)F | |
| 7582- | 7582-0307 | | 824980- | O═[S](═O)(N(CC(═O) | C19H25N3O3S2 |
| 0307 | | | 59-8 | NCC[S]C/C1═C/C═CC═C1) | |
| | | | | C2═CC═CC═C2)N(C)C | |
| C336- | C336-0168 | | 959484- | N(C(═N1)C(═C2C═C3) | C28H22FN5O5S |
| 0168 | | | 31-2 | C═C3)(C(═N2)SCC(NC(═C | |
| | | | | (F)C2)C═CC═2)═O) | |
| | | | | C(C1CC(NCC1═CC(═C | |
| | | | | (O2)C═C1)OC2)═O)═O | |
| T3671 | Vitexin-2″-O- | Vitexin-2-O- | 64820- | C[C@@H]1O[C@@H] | C27H30O14 |
| | rhamnoside | rhaMnoside; 2″- | 99-1 | (O[C@@H]2[C@@H] | |
| | | O- | | (O)[C@H](O)[C@@H] | |
| | | Rhamnosylvitexin; | | (CO)O[C@H]2c2c(O)cc | |
| | | Apigenin-8- | | (O)c3c2oc(cc3═O)—c2ccc | |
| | | C-glucoside | | (O)cc2)[C@H](O) | |
| | | | | [C@H](O)[C@H]1O | |
| G205- | G205-0745 | | 951489- | N(C(S1)═N2)(N═C1N(CC1) | C21H25N5O4S |
| 0745 | | | 25-1 | CCO1)C(C(═C2)C(NCC(═CC1) | |
| | | | | C═CC═1OCCCC)═O)═O | |
| Y041- | Y041-2269 | | | O═C4OC3═CC(OCC(═O) | C26H26N2O7 |
| 2269 | | | | N[C@]([H])(C/C1═C/ | |
| | | | | NC═2C═CC(O)═CC1═2) | |
| | | | | C(═O)O)═CC═C3C | |
| | | | | (═C4)CCCC | |
| 7680- | 7680-2228 | | 838252- | O═[S](═O)(N(CC(═O) | C20H26N4O5S2 |
| 2228 | | | 73-6 | N/C1═C/C═C(C═C1) | |
| | | | | [S](═O)(═O)N2CCCC2) | |
| | | | | C═3C═CC═CC═3)N(C)C | |
| C200- | C200-1101 | | 1037289- | O═C1N4C(═N[C@@] | C28H25ClN4O2S |
| 1101 | | | 06-7 | 1([H])CCC(═O)NCC/C2═C/ | |
| | | | | C═CC═C2)C═3C═CC═CC═3/ | |
| | | | | N═C4/[S]CC═5C═CC | |
| | | | | ([Cl])═CC═5 | |
| T7142 | Cephalosporin | | 59143- | [Zn++]•[H][C@@] | C16H19N3O8SZn |
| | C zinc salt | | 60-1 | 12SCC(CC(C)═O)═C(N1C(═O) | |
| | | | | [C@H]2NC(═O)CCC | |
| | | | | [C@@H](N)C | |
| | | | | ([)—]═O)C([O—])═O | |
| G856- | G856-4196 | | | O═C(N/C1═C/C═C(/C) | C28H30N4O4 |
| 4196 | | | | C═C1)C(═O)NC[C@@] | |
| | | | | ([H])(N2CCN(CC2) | |
| | | | | C═3C═CC═CC═3) | |
| | | | | C5═CC═4OCOC═4C═C5 | |
| T2402 | Tianeptine | Tianeptine | 30123- | CN1c2ccccc2C(c2c | C21H24ClN2NaO4S |
| | sodium | sodium salt | 17-2 | (S1(═O)═O)cc(cc2)Cl) | |
| | | | | NCCCCCCC(═O) | |
| | | | | [O—]•[Na+] | |
| C906- | C906-0334 | | 890800- | C(═C1N(CC2)CC2)(C | C25H33N3O3 |
| 0334 | | | 06-3 | (C1═O)═O)NCC(CC1) | |
| | | | | CCC1C(═O)NCCC | |
| | | | | (C═C1)═CC═C1C | |
| T2923 | Apremilast | CC-10004 | 608141- | CCOc1c(ccc(c1)[C@@H] | C22H24N2O7S |
| | | | 41-9 | (CS(═O)(═O)C)N1C | |
| | | | | (═O)c2c(C1═O)c(ccc2) | |
| | | | | NC(═O)C)OC | |
| Y050- | Y050-1938 | | 878423- | O═[S](═O)(N(C)CC(═O) | C19H22N2O6S |
| 1938 | | | 88-2 | NCC═2C═C1OCOC1═CC═2) | |
| | | | | C═3C═CC(═CC═3)OCC | |
| T3879 | Silychristin | Silicristin | 33889- | COc1cc(ccc1O)[C@@H] | C25H22O10 |
| | | | 69-9 | 1Oc2c(cc(cc2O)[C@H] | |
| | | | | 2Oc3cc(O)cc(O)c3C(═O) | |
| | | | | [C@@H]2O)[C@H]1CO | |
| T1227 | Cepazine | Cefuroxime | 64544- | CO\N═C(/C(═O)N | C20H22N4O10S |
| | | axetil; Ceftin; | 07-6 | [C@H]1[C@H] | |
| | | Zinnat; Elobact | | 2SCC(COC(N)═O)═C | |
| | | | | (N2C1═O)C(═O)OC(C)OC | |
| | | | | (C)═O)c1ccco1 | |
| K786- | K786-6600 | | 697781- | N(N═C1C(═CC2) | C24H30N4O2 |
| 6600 | | | 60-5 | C═CC═2)(C(CC1)═O)CC | |
| | | | | (═O)NCCCN(C(═CC1C) | |
| | | | | C═CC═1)CC | |
| E587- | E587-0421 | | 878055- | N1(C(═C(C(═C1)SCC | C26H31N3O3S |
| 0421 | | | 77-7 | (NC(═C(OCC)C1) | |
| | | | | C═CC═1)═O)C1) | |
| | | | | C═CC═1)CC(N(CCC1)CCC1)═O | |
| C651- | C651-0859 | | 902582- | N(OC1C(NC2C═C(C | C20H22N2O6 |
| 0859 | | | 18-7 | (═CC═2)OC)OC)═O)═C | |
| | | | | (C1)C(C═C(C(═C1)OC)OC)═C1 | |
| T1066 | Ketanserin | R41468; | 74050- | Fc1ccc(cc1)C(═O) | C22H22FN3O3 |
| | | Ketanserinum; | 98-9 | C1CCN(CCn2c(═O)[nH] | |
| | | Ketanserin | | c3ccccc3c2═O)CC1 | |
| | | tartrate | | | |
| T1331 | Flavin | Riboflavin 5′- | 130-40-5 | [Na+]•c12c(nc3c(n1C | C17H20N4NaO9P |
| | mononucleotide | phosphate | | [C@@H]([C@@H] | |
| | | sodium; Vitamin | | ([C@@H](COP(═O)([O—]) | |
| | | B2 Phosphate | | O)O)O)O)cc(c(C)c3) | |
| | | Sodium | | C)c(═O)[nH]c(═O)n2 | |
| | | Salt; FMN- | | | |
| | | Na; Riboflavin | | | |
| | | phosphate | | | |
| | | sodium; | | | |
| | | riboflavin-5′- | | | |
| | | phosphate; FMN | | | |
| E465- | E465-0564 | | 891924- | S(N(CC1)CCC1C(═O)OCC) | C17H22N2O5S2 |
| 0564 | | | 26-8 | (C(═CC(NC(C1C)═O)═C | |
| | | | | (S1)C1)C═1)(═O)═O | |
| T7100 | PLX-5622 | | 1303420- | COc1ncc(F)cc1CNc1ccc | C21H19F2N5O |
| | | | 67-8 | (Cc2c[nH]c3ncc(C) | |
| | | | | cc23)c(F)n1 | |
| E986- | E986-1019 | | 894894- | O═C(CCC(═O)NC/C1═C/ | C26H27N3O6 |
| 1019 | | | 84-9 | C(═C/C═C1/OC)OC) | |
| | | | | N4CC═2C═C/C═C(/OC) | |
| | | | | C═2OC3═NC═CC═C34 | |
| C700- | C700-1423 | | 902589- | O═[S](═O)(CC═1N═C | C19H24ClN3O5S |
| 1423 | | | 58-6 | (OC═1C)C2═CC | |
| | | | | ([Cl])═CC═C2)CC | |
| | | | | (═O)NCCN3CCOCC3 | |
| C636- | C636-1184 | | 1037192- | O═C2N(C(═O)[C@@] | C27H31N3O5 |
| 1184 | | | 50-9 | ([H])(CC(═O)N/C1═C/ | |
| | | | | C═C(C═C1)OC)N2CC/ | |
| | | | | C3═C/CCCC3)C4═CC | |
| | | | | (OC)═CC═C4 | |
| E461- | E461-0614 | | 872840- | C1(═C(N(C(═N1)N(C1) | C20H23N5O4 |
| 0614 | | | 60-3 | C(═CC2)C═CC═2CC) | |
| | | | | C1)C1═O)N(C(N1CC | |
| | | | | (═O)OCC)═O)C | |
| E461- | E461-0573 | | 300394- | C1(═C(N(C(═N1)N(C1) | C19H21N5O4 |
| 0573 | | | 36-9 | C(═CC2)C═CC═2C) | |
| | | | | C1)C1═O)N(C(N1CC(═O) | |
| | | | | OCC)═O)C | |
| T5171 | Treprostinil | UT-15 | 289480- | [Na+]•CCCCC[C@H] | C23H33NaO5 |
| | Sodium | | 64-4 | (O)CC[C@H]1[C@H] | |
| | | | | (O)C[C@@H]2Cc3c(C | |
| | | | | [C@H]12)cccc3OCC | |
| | | | | ([O—])═O | |
| T1503 | Esmolol | Esmolol | 81161- | c1(ccc(cc1)CCC(═O) | C16H25NO4•HCl |
| | hydrochloride | HCl; ASL8052 | 17-3 | OC)OCC(CNC(C)C)O•Cl | |
| T3886 | Rosavin | Rosavidin | 84954- | O1[C@H]([C@@H]([C@H] | C20H28O10 |
| | | | 92-7 | 1CO[C@@H]1OC[C@@H] | |
| | | | | ([C@@H]([C@H]1O) | |
| | | | | O)O)O)O)O)OC/C═C/c1ccccc1 | |
| G948- | G948-4026 | | 933019- | O═[S](═O)(C═1C═C | C24H28N4O4S |
| 4026 | | | 97-7 | (C═CC═1C)C═2/N═C(/C) | |
| | | | | ON═2)N3CCC([H]) | |
| | | | | (CC3)C(═O)NC═4C═CC | |
| | | | | (C)═CC═4C | |
| T2911 | Stevioside | | 57817- | C[C@@]12CCC[C@] | C38H60O18 |
| | | | 89-7 | (C)([C@H]1CC[C@@] | |
| | | | | 13CC(═C)[C@@](C1) | |
| | | | | (CC[C@@H]23)O | |
| | | | | [C@@H]1O[C@H] | |
| | | | | (CO)[C@@H](O)[C@H] | |
| | | | | (O)[C@H]1O[C@@H] | |
| | | | | 1O[C@H](CO)[C@@H] | |
| | | | | (O)[C@H](O)[C@H] | |
| | | | | 1O)C(═O)O[C@@H] | |
| | | | | 1O[C@H](CO)[C@@H] | |
| | | | | (O)[C@H](O)[C@H]1O | |
| C700- | C700-1219 | | 902443- | S(C(C═C(N(CC(N(CC1) | C28H36N4O5S |
| 1219 | | | 53-2 | CCN1C(═C(C1C)C) | |
| | | | | C═CC═1)═O)C1═O) | |
| | | | | C(OC1)═C1)═C1)(N | |
| | | | | (CCC1)CCC1)(═O)═O | |
| Y031- | Y031-0649 | | 725693- | S(NCC(OC1)CC1)(C | C18H19FN2O4S |
| 0649 | | | 58-3 | (C═C1)═CC═C1NC(C(═CC1F) | |
| | | | | C═CC═1)═O)(═O)═O | |
| T6085 | PF543 | PF 543; PF-543; | 1415562- | Cc1cc(CS(═O)(═O) | C27H31NO4S |
| | | Sphingosine | 82-1 | c2ccccc2)cc(OCc2ccc | |
| | | Kinase 1 | | (CN3CCC[C@@H] | |
| | | Inhibitor II | | 3CO)cc2)c1 | |
| Y031- | Y031-0429 | | 725693- | S(NCC(OC1)CC1)(C | C18H19FN2O4S |
| 0429 | | | 41-4 | (C═C1)═CC═C1NC(C | |
| | | | | (═CC1)C═CC═1F)═O) | |
| | | | | (═O)═O | |
| T3899 | Calceolarioside | Nuomioside A; | 105471- | C1═CC(═C(C═C1CCO | C23H26O11 |
| | B | Desrhamnosyl | 98-5 | [C@H]2[C@@H] | |
| | | isoacteoside | | ([C@H]([C@@H] | |
| | | | | ([C@H](O2)COC(═O)/C═C/ | |
| | | | | C3═CC(═C(C═C3)O) | |
| | | | | O)O)O)O)O)O | |
| C880- | C880-0694 | | 906243- | O═C1C4═C(C═NN1CC | C26H27N5O3 |
| 0694 | | | 33-2 | (═O)NCCCN2CCCC2═O) | |
| | | | | C3═CC═CC═C3N4C/ | |
| | | | | C5═C/C═CC═C5 | |
| T6846 | Vesatolimod | GS-9620 | 1228585- | CCCCOc1nc2c(c(n1) | C22H30N6O2 |
| | | | 88-3 | N)NC(═O)CN2Cc1cc | |
| | | | | (ccc1)CN1CCCC1 | |
| D715- | D715-0611 | | 956040- | C(O1)(C(C(═C(C1═O) | C25H25NO6 |
| 0611 | | | 89-4 | C1)CCC1)═C1)═C(C | |
| | | | | (OCC(NC(C(═O)O)CC | |
| | | | | (═CC2)C═CC═2)═O)═C1)C | |
| D134- | D134-0324 | | 876725- | O═C(NCC═1OC═CC═1) | C18H19N3O4 |
| 0324 | | | 25-6 | CCC/C2═N/C(═NO2) | |
| | | | | C3═CC(OC)═CC═C3 | |
| Y031- | Y031-2258 | | 838871- | N1C═C(C(C1═CC═C | C19H19BrN2O2 |
| 2258 | | | 13-9 | 1OC)═C1)CCNC(═O) | |
| | | | | CC(C═C1)═CC═C1[Br] | |
| T7413 | JNJ-5207852 | | 398473- | C(COc1ccc(CN2CCC | C20H32N2O |
| | | | 34-2 | CC2)cc1)CN1CCCCC1 | |
| T0342 | Carvedilol | BM 14190 | 610309- | O•OP(O)(O)═O•OP(O) | C24H26N2O4•H2O•H3O4P |
| | phosphate | (phosphate | 89-2 | (O)═O•COc1ccccc1OCCNCC | |
| | | hemihydrate); | | (O)COc1cccc2[nH] | |
| | | Carvedilol | | c3ccccc3c12•COc1ccccc1OCCNCC | |
| | | phosphate | | (O)COc1cccc2[nH] | |
| | | hemihydrate | | c3ccccc3c12 | |
| D389- | D389-0267 | | 848205- | C1(═C(N(C(═N1)N(C | C26H27N5O4 |
| 0267 | | | 63-0 | (C═C(C(═C1)C)C)═C1) | |
| | | | | C1)CC1)C1═O)N(C | |
| | | | | (N1CC(═O)OCC(═CC1) | |
| | | | | C—CC═1)═O)C | |
| 6913- | 6913-0019 | | 909860- | C(═C(N1C)C)(C | C22H32N2O4 |
| 0019 | | | 72-6 | (C1═C1)═CC(OCC(CN | |
| | | | | (CC2)CCC2C)O)═C1)C | |
| | | | | (═O)OCC | |
| T6S0119 | Dauricine | | 524-17-4 | COc1cc2CCN(C)[C@H] | C38H44N2O6 |
| | | | | (Cc3ccc(Oc4cc(C[C@H] | |
| | | | | 5N(C)CCc6cc(OC)c(OC) | |
| | | | | cc56)ccc4O)cc3)c2cc1OC | |
| T7183 | CPI-444 | V81444; | 1202402- | Cc1ccc(o1)—c1nc | C20H21N7O3 |
| | | ciforadenant | 40-1 | (N)nc2n(Cc3cccc(CO | |
| | | | | [C@H]4CCOC4)n3)nnc12 | |
| G008- | G008-5517 | | 894918- | S(C(═CC1C(═C(C2C) | C26H33N3O5S |
| 5517 | | | 95-7 | C)ON═2)C(═CC═1) | |
| | | | | OC)(NCC(CC1)CCN1CC(═C | |
| | | | | (OC)C1)C═CC═1)(═O)═O | |
| T0179 | Ticagrelor | AZD6140; AR-C | 274693- | CCCSc1nc2c(nnn2[C@@H] | C23H28F2N6O4S |
| | | 126532XX | 27-5 | 2C[C@H](OCCO)[C@@H] | |
| | | | | (O)[C@H]2O)c(N[C@@H] | |
| | | | | 2C[C@H]2c2cc(F)c(F)cc2)n1 | |
| E977- | E977-0894 | | 894887- | O═[S](═O)(NCCC(═O) | C21H25N3O5S |
| 0894 | | | 97-9 | NCC═1C═CC(═CC═1) | |
| | | | | OC)C═3C═C2CCCC | |
| | | | | (═O)NC2═CC═3 | |
| 6655- | 6655-0470 | | 760196- | O═C(/C2═C/N(CCO/ | C22H19NO2S |
| 0470 | | | 67-6 | C1═C/C═C(/C)C═C1) | |
| | | | | C═3C═CC═CC2═3) | |
| | | | | C═4[S]C═CC═4 | |
| T4S0998 | Trifolirhizin | | 6807-83- | OC[C@H]6O[C@@H] | C22H22O10 |
| | | | 6 | (Oc1cc2OC[C@H] | |
| | | | | 3c4cc5OCOc5cc4O[C@H] | |
| | | | | 3c2cc1)[C@H](O) | |
| | | | | [C@@H](O)[C@@H]6O | |
| T5S1094 | Forsythoside E | | 93675- | C[C@@H]1O[C@@H] | C2OH30O12 |
| | | | 88-8 | (OC[C@H]2O[C@@H] | |
| | | | | (OCCc3ccc(O)c(O)c3) | |
| | | | | [C@H](O)[C@@H] | |
| | | | | (O)[C@@H]2O)[C@H] | |
| | | | | (O)[C@H](O)[C@H]1O | |
| T4602 | Hydrocortisone | Hydrocortisone | 2203-97- | [H][C@@]12CC[C@] | C25H34O8 |
| | 21- | 21- | 6 | (O)(C(═O)COC(═O) | |
| | hemisuccinate | he; | | CCC(O)═O)[C@@]1(C) | |
| | | hydrocortisone 21- | | C[C@H](O)[C@@]1 | |
| | | hemisuccinate*free | | ([H])[C@@]2([H]) | |
| | | acid | | CCC2═CC(═O)CC[C@]12C | |
| E977- | E977-0921 | | 894888- | O═[S](═O)(NCCC(═O) | C21H25N3O4S |
| 0921 | | | 70-1 | NC═1/C═C(/C)C═CC═1C) | |
| | | | | C═3C═C2CCCC(═O)NC2═CC═3 | |
| G937- | G937-2830 | | 933202- | O═[S](═O)(NCC(═O) | C22H29ClN4O5S |
| 2830 | | | 35-8 | N1CCN(CC1)C2═CC | |
| | | | | ([Cl])═CC═C2)C═3/ | |
| | | | | C(═C(/C)N(C)C═3C)C(═O)OCC | |
| F070- | F070-0397 | | 959506- | C(C(═N1)N(CC2)CCC2C | C25H27F3N4O3 |
| 0397 | | | 77-5 | (═O)NCCC2═CC(═C | |
| | | | | (C═C2)OC)OC)(═NC | |
| | | | | (C1═C1)═CC═C1)C(F)(F)F | |
| T0812 | Propranolol | Propranolol | 318-98-9 | Cl•CC(C)NCC(O) | C16H22ClNO2 |
| | hydrochloride | HCl; AY- | | COc1cccc2ccccc12 | |
| | | 64043; ICl- | | | |
| | | 45520; NCS- | | | |
| | | 91523 | | | |
| G357- | G357-1938 | | 959562- | C(N(C1═O)C2C)(═NC | C24H21N3O5S |
| 1938 | | | 33-5 | (═C1)COC(C(═C(NC | |
| | | | | (═O)COC(C | |
| | | | | (C)═C1)═CC═C1)C1) | |
| | | | | C═CC═1)═O)SC═2 | |
| 5228- | 5228-0298 | | 476481- | C(═C(C1═O)N(C2SCC | C19H24N4O3S |
| 0298 | | | 85-3 | (O)C)CCCC(═CC3) | |
| | | | | C═CC═3)(N═2)N(C | |
| | | | | (N1C)═O)C | |
| E545- | E545-0290 | | 892427- | O═C3N(CCCC)C(═O) | C22H22N2O3 |
| 0290 | | | 14-4 | C═2OC═1C═CC═CC═1C═2N3C/ | |
| | | | | C4═C/C═C(/C)C═C4 | |
| T1959 | BIX 01294 | BIX 01294 | 1392399- | Cl•COc1c(OC)cc2c | C28H38N6O2•3HC1 |
| | Trihydrochloride | | 03-9 | (NC3CCN(Cc4ccccc4)CC3) | |
| | | | | nc(nc2c1)N1CCCN(C) | |
| | | | | CC1•Cl•Cl | |
| C199- | C199-0140 | | 850935- | N(C(C(C(═C1)C2)═CC | C28H33NO6 |
| 0140 | | | 38-5 | (═C1OC)OC)COC1C═C | |
| | | | | (OC3═O)C(C(═C3) | |
| | | | | C)═CC═1)(C(═O)C | |
| | | | | (CC)CC)C2 | |
| N081- | N081-0783 | | 332849- | C(C(CC(═O)NCCC1C═C | C20H21NO5 |
| 0783 | | | 41-9 | (C(O2)═CC═1)OC2) | |
| | | | | CC(C═C1)═CC═C1) | |
| | | | | (═O)O | |
| C258- | C258-0605 | | 862316- | C(N(C1═O)CC(NC2C═C | C28H29N3O3S |
| 0605 | | | 96-9 | (C(═CC═2)C)C)═O) | |
| | | | | (═C(C(N1CCC(═CC1) | |
| | | | | C═CC═1)═O)C | |
| | | | | (═C1CC2)CC2)S1 | |
| C199- | C199-0115 | | 850935- | N(C(C(═CC1) | C30H29NO6 |
| 0115 | | | 47-6 | C═CC═1C)═O)(C(C | |
| | | | | (C(═C1)C2)═CC(═C1OC) | |
| | | | | OC)COC1C═C(OC3═O)C | |
| | | | | (C(═C3)C)═CC═1)C2 | |
| D011- | D011-0852 | | 883637- | O═C1C[C@@]([H]) | C27H26ClN3O3 |
| 0852 | | | 27-2 | (CN1/C2═C/C═C/C═C2/[Cl]) | |
| | | | | C4═NC3═CC═CC═C3N4CCCO/ | |
| | | | | C5═C/C═C/C═C5/OC | |
| 1349- | 1349-0007 | | 314033- | O═C(O)CCN1C | C18H18N2O3S |
| 0007 | | | 58-4 | (═NC═2C═CC═CC1═2) | |
| | | | | [S]CCOC═3C═CC═CC═3 | |
| 5782- | 5782-5442 | | 496774- | C(═CC1C(SC2)═CC═2) | C23H17NO4S |
| 5442 | | | 72-2 | (C(OCC(C | |
| | | | | (C═C2OC)═CC═C2)═O)═O) | |
| | | | | C(C(N═1)═C1)═CC═C1 | |
| C528- | C528-1116 | | 901742- | C(═NC(CS(CC(NC(C(C)C1) | C21H28N2O4S |
| 1116 | | | 07-2 | CCC1)═O)(═O)═O)═C1C) | |
| | | | | (O1)C(C(C)═C1)═CC═C1 | |
| K405- | K405-3034 | | 890884- | N(C(═C1C2NCCCC | C22H23N5 |
| 3034 | | | 23-8 | (C═C3)═CC═C3)N═CN═2) | |
| | | | | (N═C1)C(═CC(═C(C1)C)C)C═1 | |
| T5S0733 | Picroside III | | 64461- | COc1cc(\C═C\C(═O) | C25H30O13 |
| | | | 95-6 | OC[C@H]2O[C@@H] | |
| | | | | (O[C@@H]3OC═C[C@H] | |
| | | | | 4[C@H](O)[C@@H] | |
| | | | | 5O[C@]5(CO)[C@@H]34) | |
| | | | | [C@H](O)[C@@H] | |
| | | | | (O)[C@@H]2O)ccc1O | |
| T6018 | Zosuquidar | LY-335979 | 167465- | Cl•Cl•Cl•O[C@@H] | C32H31F2N3O2•3HCl |
| | 3HCl | trihydrochloride; | 36-3 | (COC1═CC═CC2═C1C═CC═N2) | |
| | | RS 33295-198 | | CN1CCN(CC1) | |
| | | trihydrochloride; | | [C@@H]1C2═C(C═CC═C2) | |
| | | Zosuquidar | | [C@@H]2[C@H] | |
| | | trihydrochloride; | | (C3═C1C═CC═C3)C2(F)F | |
| | | Zosuquidar | | | |
| | | (LY335979) | | | |
| | | 3HCl; RS | | | |
| | | 33295-198 | | | |
| | | (D06387) 3HCl | | | |
| T2018 | Apilimod | STA 5326 | 541550- | c1(nc(cc(n1)N/N═C/ | C23H26N6O2 |
| | | | 19-0 | c1cccc(c1)C)N1CCOCC1) | |
| | | | | OCCc1ncccc1 | |
| G361- | G361-0500 | | 932281- | C(N(N1)C2C)(C(N(N═2) | C24H32N6O2 |
| 0500 | | | 65-7 | CC(═O)NCCCN(C(CC) | |
| | | | | C2)CCC2)═O)═CC═1C | |
| | | | | (C═C1)═CC═C1 | |
| C450- | C450-0646 | | 1034591- | S(NC(C(NC(═C(OCC) | C24H31N3O5S |
| 0646 | | | 56-4 | C1)C═CC═1)═O)CC | |
| | | | | (C)C)(C1═CC(═C(N2C | |
| | | | | (═O)C)C═C1)CC2)(═O)═O | |
| K786- | K786-2099 | | 1037168- | O═C2N(C/C1═C/C═C | C28H30N2O4 |
| 2099 | | | 03-8 | (C═C1)CC)[C@]([H]) | |
| | | | | (C3═CC═CC═C23) | |
| | | | | C(═O)NCCC═4/C═C | |
| | | | | (/OC)C(═CC═4)OC | |
| 8388- | 8388-0819 | | | O═C(C[S]C═1OC(═N | C15H18ClN3O2S |
| 0819 | | | | N═1)[C@](N)([H]) | |
| | | | | [C@](C)([H])CC)C2═C/ | |
| | | | | C═C(/[Cl])C═C2 | |
| 3257- | 3257-2542 | | 292612- | O═C(OCC(═O)C1═C/ | C25H18ClNO3 |
| 2542 | | | 55-6 | C═C(/C)C═C1)C2═CC | |
| | | | | (═NC═3C═CC═CC2═3) | |
| | | | | C═4C═CC([Cl])═CC═4 | |
| D236- | D236-0021 | | 1008958- | C(C(CC(═O)OCCCC(═CC1) | C15H20N2O3 |
| 0021 | | | 65-3 | C═CC═1)N1)(NCC1)═O | |
| T0228 | Methyl | | 11013- | COc1ccc(cc1OC)[C@@H] | C29H36O15 |
| | hesperidin | | 97-1 | 1CC(═O)c2c(O)cc | |
| | | | | (O[C@@H]3O[C@H] | |
| | | | | (CO[C@@H]4O[C@@H] | |
| | | | | (C)[C@H](O)[C@@H] | |
| | | | | (O)[C@H]4O)[C@@H] | |
| | | | | (O)[C@H](O)[C@H]3O)cc2O1 | |
| E750- | E750-0072 | | 1037256- | N(C(N1C(CC2)CC2)═O) | C23H24FN3O3 |
| 0072 | | | 79-3 | (C(C1CC(NC(═CC1) | |
| | | | | C═CC═1F)═O)═O) | |
| | | | | C(C═C1C)═CC═C1 | |
| K279- | K279-0833 | | 1037168- | O═C1N4C(═N[C@@] | C29H28N4O3S |
| 0833 | | | 66-3 | 1([H])CCC(═O) | |
| | | | | NCC═2C═CC═CC═2OC) | |
| | | | | C═3C═CC═CC═3/ | |
| | | | | N═C4/[S]CC═5C═CC | |
| | | | | (C)═CC═5 | |
| T6633 | Ranolazine | RS 43285- | 95635- | COc1ccccc1OCC(O) | C24H33N3O4 |
| | | 003; CVT | 55-5 | CN1CCN(CC(═O)Nc2c | |
| | | 303; Ranexa | | (C)cccc2C)CC1 | |
| T5711 | Methylnissolin- | (6aR,11aR)-3- | 94367- | [H][C@@]12COc3cc | C23H26O10 |
| | 3-O- | hydroxy-9,10- | 42-7 | (O[C@@H]4O[C@H] | |
| | glucoside | diMethoxy pt | | (CO)[C@@H](O)[C@H] | |
| | | | | (O)[C@H]4O)ccc3[C@] | |
| | | | | 1([H])Oc1c2ccc(OC)c1OC | |
| D389- | D389-0835 | | 877803- | C(═C(C1═O)N(C2N(CCC | C21H25N5O4 |
| 0835 | | | 29-7 | (═CC3)C═CC═3) | |
| | | | | C3)CC3)(N═2)N(C(N1CC | |
| | | | | (═O)OCC)═O)C | |
| 8009- | 8009-9265 | | 309278- | O═[S](═O)(N1CCCCCC1) | C22H22N2O4S |
| 9265 | | | 11-3 | C═2C═C(C═CC═2)C(═O) | |
| | | | | OC═3C═CC═C4C═CC═NC═34 | |
| C795- | C795-0720 | | 903150- | N(C(C(N(N1)CC(═O) | C20H29N5O2S |
| 0720 | | | 68-5 | NCCCN(CC)CC)═O)═C2) | |
| | | | | (C(═C2S2)C═C2C)C═1CC | |
| C320- | C320-0115 | | 688759- | N(C(═N1)C(═CC2) | C21H31N5O3 |
| 0115 | | | 32-2 | C═CC═2OC)═C(O1)CCC | |
| | | | | (═O)NCCCN(CC1)CCN1CC | |
| T3540 | IMR-1A | | 331862- | COc1cc(\C═C2/SC(═S) | C13H11NO5S2 |
| | | | 41-0 | NC2═O)ccc1OCC(O)═O | |
| E544- | E544-0037 | | 892379- | C(═C(C1SCC(NC(C═C2C | C27H21F3N4O3S |
| 0037 | | | 45-2 | (F)(F)F)═CC═C2)═O) | |
| | | | | C2)(N═C(N═1)C | |
| | | | | (C—C1)═CC═C1)OC | |
| | | | | (═C2C1CO)C(═NC═1)C | |
| T2306 | Brexpiprazole | OPC-34712 | 913611- | O═c1ccc2ccc(OCCCCN3CCN | C25H27N3O2S |
| | | | 97-9 | (CC3)c3cccc4sccc34) | |
| | | | | cc2[nH]1 | |
| C796- | C796-1295 | | 959515- | O═C(C[S]/C2═N/ | C26H24ClFN4OS |
| 1295 | | | 28-7 | C1═CC═CC═C1N2CC═3/C═C | |
| | | | | (/[Cl])C═CC═3)N4CCN | |
| | | | | (CC4)C5═C/C═C(/[F])C═C5 | |
| T2491 | AZ5104 | | 1421373- | CN(C)CCN(C)c1cc(c(cc1NC | C27H31N7O2 |
| | | | 98-9 | (═O)C═C)Nc1nccc | |
| | | | | (n1)c1c[nH]c2ccccc12)OC | |
| D364- | D364-2068 | | 1147183- | N(C(N1)═O)(C(C(═C1O) | C27H29N3O4S |
| 2068 | | | 28-5 | C(═NC(═C1C2)C═CC═2) | |
| | | | | CC(S1)C(═CC1)C═CC═1OCC)═O) | |
| | | | | C(CC1)CCC1 | |
| T3S1149 | Ganoderic acid | | 98665- | C[C@H](CC(═O)CC(C) | C30H44O8 |
| | G | | 22-6 | C(O)═O)[C@H]1CC | |
| | | | | (═O)[C@@]2(C) | |
| | | | | C3═C(C(═O)[C@@H] | |
| | | | | (O)[C@]12C)[C@@] | |
| | | | | 1(C)CC[C@H](O)C(C)(C) | |
| | | | | [C@@H]1C[C@@H]3O | |
| C848- | C848-0215 | | 863449- | C(N═C1S(═O)(═O)CC) | C18H24N2O4S3 |
| 0215 | | | 34-7 | (═C(S1)N(CC1)CCClC)S(C | |
| | | | | (C═C1)═CC═C1C)(═O)═O | |
| T0696 | Naftopidil | BM-15275; KT- | 57149- | COc1ccccc1N1CCN(CC(O) | C24H28N2O3 |
| | | 611 | 07-2 | COc2cccc3ccccc23)CC1 | |
| G856- | G856-5036 | | 877642- | O═[S](═O)(NCC/C2═C/ | C21H20N4O5S2 |
| 5036 | | | 30-3 | [S]C1═NC(═NN12) | |
| | | | | C3═CC═C(C═C3)OC) | |
| | | | | C5═CC═4OCCOC═4C═C5 | |
| 8211- | 8211-0295 | | 892720- | C(═C(N1)C)(C(CN(CC2)CCN2C | C21H22ClN3O |
| 0295 | | | 69-3 | (═CC2)C═CC═2[Cl])═O) | |
| | | | | C(C1═C1)═CC═C1 | |
| T6078 | Saracatinib | AZD0530 | 379231- | CN1CCN(CCOc2cc | C27H32ClN5O5 |
| | | | 04-6 | (OC3CCOCC3)c3c | |
| | | | | (Nc4c5OCOc5ccc4Cl) | |
| | | | | ncnc3c2)CC1 | |
| 5678- | 5678-0009 | | 685122- | C(N═C1C(═C(F)C2) | C22H16ClFN2O3S |
| 0009 | | | 34-3 | C═CC═2)(S(C(═CC2) | |
| | | | | C═CC═2[Cl])(═O)═O)═ | |
| | | | | C(O1)NCC(C═C1)═CC═C1 | |
| G408- | G408-1998 | | 932331- | S(NC(C═C1)═CC═C1C | C2OH18N2O4S |
| 1998 | | | 05-0 | (═O)C)(C(═CC1) | |
| | | | | C═CC═1C(OC1C | |
| | | | | (C2)C2)═CN═1)(═O)═O | |
| T2320 | Indacaterol | | 312753- | CCc1c(cc2CC(Cc2c1) | C24H28N2O3 |
| | | | 06-3 | NC[C@@H](c1c2ccc | |
| | | | | (═O)[nH]c2c(cc1)O)O)CC | |
| 4341- | 4341-0035 | | 431934- | O═C2N(CCCN1CCOCC1) | C26H30N2O6 |
| 0035 | | | 42-8 | [C@@]([H])(C | |
| | | | | (═C2O)C(═O)C3═CC═C | |
| | | | | (C═C3)OC)C4═CC═C | |
| | | | | (C═C4)OC | |
| G071- | G071-0411 | | 895100- | O═[S]3(═O)N(C/C1═C/C═C/ | C26H20F2N4O3S2 |
| 0411 | | | 88-6 | C═C1/C)C2═CC═CC═C2C═4N═C | |
| | | | | (N═CC3═4)[S]CC(═O)N/ | |
| | | | | C5═C/C([F])═C/C═C5/[F] | |
| T4302 | iCRT3 | | 901751- | O═C(NCCC1═CC═CC═C1) | C23H26N2O2S |
| | | | 47-1 | CSCC2═C(C)OC | |
| | | | | (C3═CC═C(CC)C═C3)═N2 | |
| T6091 | CP673451 | CP 673451; CP- | 343787- | N1(CCC(CC1)N)c1cccc2ccc | C24H27N5O2 |
| | | 673451 | 29-1 | (nc12)n1cnc2c1ccc | |
| | | | | (c2)OCCOC | |
| 6872- | 6872-0294 | | 847159- | O═C3C═2OC═1C═CC═CC═1C | C25H26N2O5 |
| 0294 | | | 57-3 | (═O)C═2[C@]([H]) | |
| | | | | (N3CCCN4CCOCC4) | |
| | | | | C═5/C═C(/OC)C═CC═5 | |
| G361- | G361-0499 | | 932335- | C(N(N1)C2C)(C(N(N═2) | C22H28N6O2 |
| 0499 | | | 20-1 | CC(═O)NCCCN(CC2) | |
| | | | | CCC2)═O)═CC═1C | |
| | | | | (C═C1)═CC═C1 | |
| E570- | E570-2192 | | 892751- | S(C1═CC2═C(N(C(C2)C) | C21H31N3O4S |
| 2192 | | | 50-7 | C(═O)C)C═C1)(═O) | |
| | | | | (═O)NCCC(NC(CCC1)CCC1)═O | |
| K788- | K788-6635 | | 899198- | O═C1CCC(═NN1CCC | C25H31N3O4 |
| 6635 | | | 29-9 | (═O)NCCC═2/C═C | |
| | | | | (/OC)C(═CC═2)OC) | |
| | | | | C3═CC═C(C═C3)CC | |
| T4050 | GSK2981278 | ROR gama | 1474110- | OCc1cc(S(═O)(═O)N | C25H35NO5S |
| | | modulator 1 | 21-8 | (CC(C)C)c2ccc(CC)cc2) | |
| | | | | ccc1OCC1CCOCC1 | |
| D345- | D345-0032 | | 1207659- | N(C(COC(═CC1) | C20H25N3O3 |
| 0032 | | | 19-5 | C═CC═1OC)═O)(CC1) | |
| | | | | CCN1CCC(N═C1)═CC═C1 | |
| D072- | D072-1432 | | 380190- | O═[S](═O)(C═1N═C | C23H26ClN3O4S |
| 1432 | | | 61-4 | (OC═1NCCCN2CCOCC2) | |
| | | | | C═3C═CC═CC═3[Cl]) | |
| | | | | C═4C═CC(C)═CC═4 | |
| 2191- | 2191-2709 | | 312718- | O═C2NC(C)═C(C(═O) | C22H24N2O6 |
| 2709 | | | 81-3 | OCC═1C═CC(═CC═1) | |
| | | | | OC)[C@]([H])(N2) | |
| | | | | C3═CC(═C/C═C3/OC)OC | |
| T3274 | S49076 | | 1265965- | O═C1N(Cc2cc3c(NC(═O)/C/ | C22H22N4O4S |
| | | | 22-7 | 3═C\c3cc(CN4CCOCC4) | |
| | | | | c[nH]3)cc2)C(═O)CS1 | |
| C276- | C276-0156 | | 725687- | C(C(═C1C(OC(C)C)═O) | C21H25NO5 |
| 0156 | | | 57-0 | C)(═C(N1)C1)C(CC1C | |
| | | | | (═CC(═C(C1)OC) | |
| | | | | OC)C═1)═O | |
| C891- | C891-1711 | | 890786- | O═C1C═4C(C═NN1CC | C23H28FN5O3 |
| 1711 | | | 93-3 | (═O)NCCN2CCOCC2)═C | |
| | | | | (C)N(C/C3═C/C═C | |
| | | | | (/[F])C═C3)C═4C | |
| G678- | G678-0075 | | 904826- | S(N(CC1)CCN1C(═CC1 | C22H28ClN3O5S |
| 0075 | | | 87-5 | [Cl])C═CC═1)(CCNC | |
| | | | | (═O)COC(═CC1)C═CC═1OCC) | |
| | | | | (═O)═O | |
| K784- | K784-6292 | | 1028072- | N(C(C1)═O)(C(C(N1C | C25H29FN2O3 |
| 6292 | | | 39-0 | (CCC1)CCC1)═O)C | |
| | | | | (C═C1)═CC═C1F)CC | |
| | | | | (═CC1)C═CC═1OC | |
| 8104- | 8104-06075 | | 880808- | CN1N═N/N═C1/[S]CCN | C20H25N5O2S |
| 06075 | | | 69-5 | ([H])C/C3═C/C | |
| | | | | (OC)═C(OC/C2═C/C═C/ | |
| | | | | C═C2/C)C═C3 | |
| T1533 | Valgancic1ovir | Valgancic1ovir | 175865- | Cl•CC(C)[C@H](N)C | C14H23ClN6O5 |
| | hydrochloride | HCl; Valcyte; | 59-5 | (═O)OCC(CO)OCn1cnc2c1 | |
| | | Valcyt | | [nH]c(N)nc2═O | |
| K786- | K786-7206 | | 697781- | O═[S](═O)(N1CC[C@@] | C23H35N3O7S |
| 7206 | | | 61-6 | 2(CC1)OCCO2)N3CC[C@] | |
| | | | | ([H])(CC3)C(═O)NCC/ | |
| | | | | C4═C/C(OC)═C(C═C4)OC | |
| G503- | G503-0141 | | 932502- | O═[S](═O)(N/C3═C/ | C22H22N2O3S2 |
| 0141 | | | 72-2 | C═2CCCN(C(═O)C═1 | |
| | | | | [S]C═CC═1)C═2C═C3) | |
| | | | | C4═CC(C)═C(C)C═C4 | |
| C651- | C651-0863 | | 902289- | C(C(N1C(C═C2)═CC═C2)═O) | C23H24N4O5 |
| 0863 | | | 38-7 | (═C(N1C)C)NC(C(ON1) | |
| | | | | CC═1C(C═C(C(═C1) | |
| | | | | OC)OC)═C1)═O | |
| 1630- | 1630-1629 | | | O═C2CC(C)(C)CC═1NC(C)═C(C | C24H32N2O4 |
| 1629 | | | | (═O)OCCOC)[C@@] | |
| | | | | ([H])(C═12)C3═CC═C | |
| | | | | (C═C3)N(C)C | |
| T2795 | Amygdalin | Laetrile | 29883- | c1ccc(cc1)[C@H] | C20H27NO11 |
| | | | 15-6 | (C#N)O[C@H]1[C@@H] | |
| | | | | ([C@H]([C@@H] | |
| | | | | ([C@H](O1)CO[C@H] | |
| | | | | 1[C@@H]([C@H]([C@@H] | |
| | | | | ([C@H](O1)CO)O)O)O)O)O)O | |
| K279- | K279-1018 | | 1037222- | N(C(═N1)C(═C2C═C3) | C30H35N5O3S |
| 1018 | | | 80-2 | C═C3)(C(═N2)SCC | |
| | | | | (NC(C═C2)═CC═C2C | |
| | | | | (C)C)═O)C(C1CCC(NC | |
| | | | | (CC1)CCC1)═O)═O | |
| T0990 | Droperidol | Dehydrobenzperidol; | 548-73-2 | Fc1ccc(cc1)C(═O) | C22H22FN3O2 |
| | | NSC | | CCCN1CCC(═CC1)n1c | |
| | | 169874 | | (═O)[nH]c2ccccc12 | |
| C919- | C919-0592 | | 890832- | C(N(N1)C(═C(C2C)CCC | C26H31N5O2 |
| 0592 | | | 00-5 | (═O)NCCC(═CC3) | |
| | | | | C═CC═3OCC)C)(═C | |
| | | | | (C═1N1)C(═CC═1C)C) | |
| | | | | N═2 | |
| T0216 | Eletriptan HBr | Eletriptan | 177834- | CN1CCC[C@@H]1Cc1c | C22H27BrN2O2S |
| | | hydrobromide; U | 92-3 | [nH]c2c1cc(CCS(═O) | |
| | | K-116044 | | (═O)c1ccccc1)cc2•Br | |
| C328- | C328-0251 | | 851860- | O(C(═N1)SCC(N(CCC2) | C18H21FN4O3S |
| 0251 | | | 70-3 | CCC2)═O)C(═N1) | |
| | | | | CNC(C(═C(F)C1) | |
| | | | | C═CC═1)═O | |
| T2899 | Liquiritin | Liquiritoside; | 551-15-5 | C1[C@H](Oc2c(C1═O) | C21H22O9 |
| | | Liquiritigenin-4′- | | ccc(c2)O)c1ccc(cc1) | |
| | | O-glucoside | | O[C@H]1[C@@H] | |
| | | | | ([C@H]([C@@H] | |
| | | | | ([C@H](O1)CO)O)O)O | |
| 8011- | 8011-7222 | | 380581- | C(C(O1)═O)(C(═O)OCC) | C17H2ON2O4S |
| 7222 | | | 14-6 | (CC1CSC(═NC(═C1C═C2) | |
| | | | | C═C2)N1)CC | |
| K786- | K786-6703 | | 898216- | O═C2COC1═CC═CC═C1N2CC | C24H29N3O3 |
| 6703 | | | 05-2 | (═O)NCCN4CCC([H])(C/ | |
| | | | | C3═C/C═CC═C3)CC4 | |
| T6853 | GSK591 | GSK3203591; | 1616391- | O═C(NC[C@H](O) | C22H28N4O2 |
| | | EPZ015866 | 87-7 | CN1Cc2ccccc2CC1)c1ccnc | |
| | | | | (NC2CCC2)c1 | |
| K221- | K221-2578 | | 896371- | O═C2NC═1C═C | C26H29ClN4O5 |
| 2578 | | | 47-4 | (C═CC═1C(═O)N2CCCCCC | |
| | | | | (═O)N3CCN(CC3)C═4C═CC | |
| | | | | ([Cl])═CC═4)C(═O)OC | |
| D345- | D345-0054 | | | O═C(N2CCN | C21H26ClN3O2 |
| 0054 | | | | (CCC═1N═CC═CC═1) | |
| | | | | CC2)[C@](C)([H])OC═3/ | |
| | | | | C═C(/C)C([Cl])═CC═3 | |
| K906- | K906-2514 | | 899717- | C(═C1N(CC2)CCC2C) | C30H36N4O3 |
| 2514 | | | 59-0 | (C(C1═O)═O)NCC | |
| | | | | (C═C1)═CC═C1C(N | |
| | | | | (CC1)CCN1C(C(═C1C) | |
| | | | | C)═CC═C1)═O | |
| T6S0444 | Salvianolic | Dan Phenolic | 96574- | OC(═O)[C@@H] | C26H22O10 |
| | acid A | Acid A | 01-5 | (Cc1ccc(O)c(O)c1)OC(═O)\ | |
| | | | | C═C\c1ccc(O)c(O)c1 | |
| K784- | K784-6600 | | 1035375- | N(C(C(N(C1)C(CCC2) | C26H34N2O5 |
| 6600 | | | 06-4 | CCC2)═O)C(C═C(C | |
| | | | | (═C2)OCCC)OC)═C2) | |
| | | | | (C1═O)CC(OC1)═CC═1 | |
| G242- | G242-0650 | | 932283- | O═[S](═O)(NC/C1═C/ | C20H27N3O3S |
| 0650 | | | 47-1 | C═C/C═C1/C)C═2/C | |
| | | | | (═C(/C)NC═2C)C(═O) | |
| | | | | N3CCCCC3 | |
| K279- | K279-1177 | | 958612- | O═C1N4C(═N[C@@] | C25H25ClN4O2S |
| 1177 | | | 96-9 | 1([H])CC(═O)N[C@] | |
| | | | | 2([H])CCCCC2) | |
| | | | | C═3C═CC═CC═3/N═C4/ | |
| | | | | [S]CC═5C═CC([Cl])═CC═5 | |
| E750- | E750-0832 | | 1032119- | O═C2N(C(═O)[C@@] | C25H23N3O6 |
| 0832 | | | 53-1 | ([H])(CC(═O)NC═1C═C | |
| | | | | (C═CC═1)C(═O) | |
| | | | | OCC)N2CC═3OC═CC═3) | |
| | | | | C═4C═CC═CC═4 | |
| G069- | G069-0064 | | 959519- | O═[S](═O)(CCC(═O) | C23H27N3O5S |
| 0064 | | | 68-7 | N1CCN(CC1)C2═CC | |
| | | | | (OC)═CC═C2)C4═CC═3CC | |
| | | | | (═O)N(C)C═3C═C4 | |

| ID | Mol Wt | Target | Bioactivity | Total_Score |
|---|---|---|---|---|
| T2934 | 584.68 | Endogenous | Bilirubin is a principal pigment of bile and one of | 3CLpro (8.96); |
| | | Metabolite | the major end products of hemoglobin | nsp16(9.16) |
| | | | decomposition. | |
| T3281 | 489.01 | RAAS | Delapril is a prodrug; it is converted into two | 3CLpro (8.3) |
| | | inhibitor | active metabolites, 5-hydroxy delapril diacid and | |
| | | | delapril diacid. These metabolites bind | |
| | | | completely to and inhibit angiotensin-converting | |
| | | | enzyme (ACE), hence blocking angiotensin I to | |
| | | | angiotensin II conversion. | |
| T5014 | 352.47 | Prostaglandin | Prostaglandin E2 is a hormone-like substance that | 3CLpro (8.19) |
| | | Receptor; End | participate in a wide range of body functions such | |
| | | ogenous | as the contraction and relaxation of smooth | |
| | | Metabolite | muscle, the dilation and constriction of blood | |
| | | | vessels, control of blood pressure, and | |
| | | | modulation of inflammation. | |
| T6109 | 666.77 | Phospholipase | Darapladib(IC50 = 0.25 nM) is a substituted | 3CLpro (7.97) |
| | | inhibitor | pyrimidone with inhibitory activity towards | |
| | | | lipoprotein-associated phospholipase-A2 (Lp- | |
| | | | PLA2). | |
| T0158 | 517.4 | Topoisomerase | Mitoxantrone Hydrochloride is the hydrochloride | 3CLpro (7.71); |
| | | inhibitor | salt of an anthracenedione antibiotic with | PLpro(9.22) |
| | | | antineoplastic activity. It is a type II | |
| | | | topoisomerase inhibitor. | |
| G211- | 399.5 | HEK293 | HEK293 inhibitor | 3CLpro (7.57) |
| 0291 | | | | |
| T0467 | 666.7 | PDE inhibitor | Sildenafil, a cyclic guanosine monophosphate | 3CLpro (7.44) |
| | | | (cGMP)-specific phosphodiesterase type 5 | |
| | | | (PDE5) Inhibitor, is used extensively for erectile | |
| | | | dysfunction and less commonly for pulmonary | |
| | | | hypertension. | |
| C924- | 421.52 | Cellular tumor | Cellular tumor antigen p53 inhibitor | 3CLpro (7.42) |
| 0274 | | antigen p53 | | |
| C519- | 544.76 | DNA | DNA polymerase beta inhibitor | 3CLpro (7.32) |
| 1772 | | polymerase | | |
| | | beta | | |
| C697- | 450.6 | Nonstructural | Nonstructural protein 1 inhibitor | 3CLpro (7.32) |
| 0280 | | protein 1 | | |
| T2873 | 785.01 | GSK-3 | Ginsenoside Rg2 is one of the major active | 3CLpro (7.31) |
| | | antagonist | components of ginseng, act as an NF-κB | |
| | | | inhibitor. | |
| T2310 | 465.34 | GSK-3 | CHIR-99021 (CT99021) is a GSK-3α/β inhibitor | 3CLpro (7.3) |
| | | | (IC50: 10/6.7 nM). | |
| T5S1103 | 610.75 | Antioxidant | 1. Isoliensinine, a natural phenolic | 3CLpro (7.27) |
| | | | bisbenzyltetrahydroisoquinoline alkaloid, has | |
| | | | received considerable attention for its potential | |
| | | | biological effects such as antioxidant and anti- | |
| | | | HIV activities. 2. Isoliensinine possesses an anti- | |
| | | | proliferative effect, which is related to the | |
| | | | decrease of the overexpression of growth factors | |
| | | | PDGF-beta, bFGF, proto-oncogene c-fos, c- | |
| | | | myc and hsp7. | |
| C738- | 399.49 | Guanine | Guanine nucleotide-binding protein G(s), subunit | 3CLpro (7.21) |
| 0291 | | nucleotide- | alpha inhibitor | |
| | | binding | | |
| | | protein G(s), | | |
| | | subunit alpha | | |
| D072- | 402.45 | Ataxin-2 | Ataxin-2 inhibitor | 3CLpro (7.12) |
| 0267 | | | | |
| 2995- | 431.51 | Beta- | Beta-lactamase AmpC inhibitor | 3CLpro (7.04) |
| 0491 | | lactamase | | |
| | | AmpC | | |
| T6332 | 443.52 | E1 Activating | MLN4924 is an effective and specific small | 3CLpro (7.02); |
| | | inhibitor | molecule NEDD8-activating enzyme (NAE) | nsp15(7.47) |
| | | | inhibitor (IC50: 4.7 nM). | |
| C115- | 496.57 | Arachidonate | Arachidonate 15-lipoxygenase inhibitor | 3CLpro (7.01) |
| 0510 | | 15- | | |
| | | lipoxygenase | | |
| T4961 | 732.64 | Others | NA | nsp16(10.01); |
| | | | | RdRP(12.53); |
| | | | | X |
| | | | | Domain(11.93) |
| T0772 | 742.67 | NOD-like | Troxerutin, a natural bioflavonoid, is isolated | nsp16(9.84) |
| | | Receptor | from Sophora japonica. It has many benefits and | |
| | | (NLR) | medicinal properties. | |
| T7740 | 894.81 | Protease- | Protease-Activated Receptor-4 diTFA, 2454 is | nsp16(9.61) |
| | | activated | the proteinase-activated receptor-4 | |
| | | Receptor | (PAR4)agonist | |
| T3054 | 610.75 | Autophagy | Daurisoline is a hERG inhibitor and also an | nsp16(9.6) |
| | | | autophagy blocker. | |
| T2S0257 | 532.58 | Others | Dehydroandrographolide Succinate is a | nsp16(9.53); |
| | | | traditional Chinese medicine used in the | RdRP(10.83) |
| | | | treatment of pneumonia, respiratory tract | |
| | | | infection. | |
| T5726 | 686.65 | Others | Specneuzhenide is a phenol glycoside isolated | nsp16(9.04) |
| | | | from Ligustrum sinense. Specneuzhenide | |
| | | | (Nuezhenide) possesses anti-tumor activity,has | |
| | | | anti-angiogenic and vision improvement effects. | |
| T1795 | 719.91 | Proteasome | Carfilzomib is an irreversible proteasome | nsp16(9.03) |
| | | inhibitor | inhibitor and antineoplastic agent that is used in | |
| | | | treatment of refractory multiple myeloma. | |
| T4049 | 418.57 | Glucokinase | Genz 123346 is an inhibitor of GL1 synthase that | nsp16(8.96) |
| | | inhibitor | blocks the conversion of ceramide to GL1. | |
| T6130 | 425.42 | p38 MAPK | Skepinone-L is a selective p38 mitogen-activated | nsp16(8.74) |
| | | inhibitor | protein kinase inhibitor. | |
| T6917 | 540.51 | Aromatase | Oleuropein is an antioxidant polyphenol isolated | nsp16(8.58); |
| | | inhibitor; ROS | from olive leaf. | RdRP(10.98) |
| T3099 | 562.71 | Histone | EPZ5676 has been used in trials studying the | nsp16(8.56); |
| | | Methyltransferase | treatment of Leukemia, Acute Leukemias, Acute | nsp15(6.96) |
| | | inhibitor | Myeloid Leukemia, Myelodysplastic Syndrome, | |
| | | | and Acute Lymphocytic Leukemia, among | |
| | | | others. | |
| T0447 | 406.47 | Adrenergic | Carvedilol Phosphate is the phosphate salt form | nsp16(8.55); |
| | | Receptor | of carvedilol, a racemic mixture and adrenergic | nsp15(7.47) |
| | | inhibitor; HIF | blocking agent with antihypertensive activity and | |
| | | modulator; | devoid of intrinsic sympathomimetic activity. The | |
| | | Integrin | S enantiomer of carvedilol nonselectively binds | |
| | | inhibitor; | to and blocks beta-adrenergic receptors, thereby | |
| | | NADPH | exerting negative inotropic and chronotropic | |
| | | inhibitor; | effects, and leading to a reduction in cardiac | |
| | | Others | output. In addition, both enantiomers of | |
| | | inhibitor; | carvedilol bind to and block alpha 1-adrenergic | |
| | | Potassium | receptors, thereby causing vasodilation and | |
| | | Channel | reducing peripheral vascular resistance. | |
| | | inhibitor; | | |
| | | VEGFR | | |
| | | inhibitor | | |
| T5841 | 500.55 | Prostaglandin | Travoprost is used to treat glaucoma and ocular | nsp16(8.49) |
| | | Receptor | hypertension,is a potent and selective FP | |
| | | | prostaglandin receptor agonist. | |
| T5234 | 449.62 | Others | Glycoursodeoxycholic acid is an acyl glycine and | nsp16(8.47) |
| | | | a bile acid-glycine conjugate. It is a secondary | |
| | | | bile acid produced by the action of enzymes | |
| | | | existing in the microbial flora of the colonic | |
| | | | environment. In hepatocytes, both primary and | |
| | | | secondary bile acids undergo amino acid | |
| | | | conjugation at the C-24 carboxylic acid on the | |
| | | | side chain, and almost all bile acids in the bile | |
| | | | duct, therefore, exist in a glycine conjugated | |
| | | | form. Bile acids are steroid acids found | |
| | | | predominantly in bile of mammals. | |
| T4678 | 601.69 | Others | Fmoc-Val-Cit-PAB is a linker for antibody-drug- | nsp16(8.46); |
| | | | conjugation (ADC). | RdRP(10.48) |
| C522- | 458.99 | Beta- | Beta-lactamase AmpC inhibitor | nsp16(8.29) |
| 0732 | | lactamase | | |
| | | AmpC | | |
| E859- | 484.61 | TAR DNA- | TAR DNA-binding protein 43 inhibitor | nsp16(8.27); |
| 1859 | | binding | | X |
| | | protein 43 | | Domain(10.05) |
| T3579 | 542.54 | Raf inhibitor | PLX8394 is an orally active inhibitor of | nsp16(8.23); |
| | | | serine/threonine-protein kinase B-Raf (BRAF) | nsp15(7.15) |
| | | | protein. PLX8394 can selectively bind to and | |
| | | | inhibit the activity of both wild-type and mutated | |
| | | | forms of BRAF, then inhibit the proliferation of | |
| | | | tumor cells which express mutated forms of | |
| | | | BRAF. PLX8394 appears to be effective against | |
| | | | tumors that express multiple mutated forms of the | |
| | | | kinase and may be an effective therapeutic agent | |
| | | | for tumors that are resistant to other BRAF | |
| | | | inhibitor therapies that are specific for the BRAF | |
| | | | V600E mutant. | |
| D011- | 485.59 | Nuclear factor | Nuclear factor erythroid 2-related factor 2 | nsp16(8.2) |
| 0999 | | erythroid 2- | inhibitor | |
| | | related factor | | |
| | | 2 | | |
| D126- | 479.58 | HEK293 | HEK293 inhibitor | nsp16(8.18) |
| 0074 | | | | |
| C147- | 371.49 | Cellular tumor | Cellular tumor antigen p53 inhibitor | nsp16(8.16) |
| 0154 | | antigen p53 | | |
| T2544 | 530.67 | Estrogen/prog | Bazedoxifene acetate is a novel selective estrogen | nsp16(8.12) |
| | | estogen | receptor modulator (SERM). | |
| | | Receptor | | |
| | | inhibitor | | |
| 8015- | 398.46 | Ataxin-2 | Ataxin-2 inhibitor | nsp16(8.08) |
| 6465 | | | | |
| T6162 | 416.99 | CDK inhibitor | BS-181 HCl is a highly selective CDK7 inhibitor | nsp16(8.03) |
| | | | with IC50 of 21 nM. It is more than 40-fold | |
| | | | selective for CDK7 than CDK1, 2, 4, 5, 6, or | |
| | | | 9. | |
| T6883 | 406.48 | DNA-PK | LY3023414 is an oral ATP competitive inhibitor | nsp16(8.02) |
| | | inhibitor; | of the class I PI3K isoforms, DNA-PK, and | |
| | | mTOR | mTOR. LY3023414 has been used in trials | |
| | | inhibitor; | studying the treatment of Neoplasm, Solid | |
| | | PI3K inhibitor | Tumor, COLON CANCER, BREAST | |
| | | | CANCER, and Advanced Cancer, among others. | |
| T6S1768 | 624.54 | Antioxidant | 1. Narcissoside, with synergism of B. flavum | nsp16(8.02) |
| | | | flavonoid and rutin, could be responsible for | |
| | | | stronger protection against mitochondrial induced | |
| | | | oxidative stress. | |
| T3217 | 525.08 | Epigenetic | PF-CBP1 is a highly selective inhibitor of the | nsp16(8) |
| | | Reader | bromodomain of CREB-binding | |
| | | Domain | protein(CREBBP). It inhibits CREBBP and p300 | |
| | | inhibitor | bromodomains with IC50 of 125 and 363 nM | |
| | | | respectively. | |
| E589- | 491.59 | Geminin | Geminin inhibitor | PLpro(10.32) |
| 2554 | | | | |
| G071- | 530.67 | ATP- | ATP-dependent Clp protease proteolytic subunit | PLpro(10.01) |
| 0431 | | dependent Clp | inhibitor | |
| | | protease | | |
| | | proteolytic | | |
| | | subunit | | |
| 7472- | 426.52 | Ataxin-2 | Ataxin-2 inhibitor | PLpro(9.89) |
| 0051 | | | | |
| D074- | 497.6 | Microtubule- | Microtubule-associated protein tau inhibitor | PLpro(9.83) |
| 0222 | | associated | | |
| | | protein tau | | |
| T5318 | 620.52 | Epigenetic | 666-15 is a potent and selective CREB inhibitor | PLpro(9.83) |
| | | Reader | (IC50: 81 nM). | |
| | | Domain | | |
| T3108 | 434.49 | EGFR | CUDC-101 is a potent inhibitor of HDAC, | PLpro(9.67) |
| | | inhibitor; | EGFR and HER2 with IC50s of 4.4, 2.4 and 15.7 | |
| | | HDAC | nM, respectively. | |
| | | inhibitor; | | |
| | | HER inhibitor | | |
| T2727 | 718.59 | Others | Salvianolic acid B is an active pharmaceutical | PLpro(9.56) |
| | | | compound present in Salvia miltiorrhiza, exerts a | |
| | | | neuroprotective effect in animal models of brain | |
| | | | and spinal cord injury. | |
| G114- | 433.55 | Mothers | Mothers against decapentaplegic homolog 3 | PLpro(9.45) |
| 0456 | | against | inhibitor | |
| | | decapentaplegic | | |
| | | homolog 3 | | |
| K788- | 488.01 | Beta- | Beta-lactamase AmpC inhibitor | PLpro(9.4) |
| 6101 | | lactamase | | |
| | | AmpC | | |
| G240- | 448.5 | Nuclear factor | Nuclear factor erythroid 2-related factor 2 | PLpro(9.18) |
| 0046 | | erythroid 2- | inhibitor | |
| | | related factor | | |
| | | 2 | | |
| C200- | 514.01 | Nuclear factor | Nuclear factor erythroid 2-related factor 2 | PLpro(9.12) |
| 4180 | | erythroid 2- | inhibitor | |
| | | related factor | | |
| | | 2 | | |
| K784- | 430.55 | Prelamin-A/C | Prelamin-A/C inhibitor | PLpro(9.06); |
| 0203 | | | | nsp15(6.89) |
| G406- | 487.58 | HEK293 | HEK293 inhibitor | PLpro(8.98) |
| 0489 | | | | |
| C336- | 533.65 | Inositol | Inositol monophosphatase 1 inhibitor | PLpro(8.96); |
| 0153 | | monophosphatase | | X |
| | | 1 | | Domain(10.41) |
| K786- | 332.49 | Survival | Survival motor neuron protein inhibitor | PLpro(8.92) |
| 5309 | | motor neuron | | |
| | | protein | | |
| T3952 | 424.55 | Autophagy | TPEN is a specific cell-permeable heavy metal | PLpro(8.91) |
| | | | chelator. | |
| Y050- | 398.48 | Nuclear factor | Nuclear factor erythroid 2-related factor 2 | PLpro(8.91) |
| 2147 | | erythroid 2- | inhibitor | |
| | | related factor 2 | | |
| T3112 | 718.79 | VDA inhibitor | Verteporfin, a benzoporphyrin derivative | PLpro(8.9) |
| | | | monoacid ring A, can inhibit the activity of YAP. | |
| E946- | 437.55 | Microtubule- | Microtubule-associated protein tau inhibitor | PLpro(8.88) |
| 0756 | | associated | | |
| | | protein tau | | |
| K784- | 437.95 | Cellular tumor | Cellular tumor antigen p53 inhibitor | PLpro(8.88) |
| 7502 | | antigen p53 | | |
| E642- | 496.61 | Mothers | Mothers against decapentaplegic homolog 3 | PLpro(8.83) |
| 1065 | | against | inhibitor | |
| | | decapentaplegic | | |
| | | homolog 3 | | |
| T4259 | 595.72 | Integrin | GLPG0187, a broad spectrum integrin receptor | PLpro(8.81) |
| | | | antagonist, inhibits αvβ1-integrin (IC50: 1.3 | |
| | | | nM). | |
| T3631 | 478.33 | PDE inhibitor | PF-8380 is an effective and orally available | PLpro(8.79) |
| | | | autotaxin inhibitor (IC50: 2.8 nM, in isolated | |
| | | | enzyme assay; 101 nM, in the human whole | |
| | | | blood). It modulates lysophosphatidic acid (LPA) | |
| | | | levels in vivo/vitro by directly inhibiting | |
| | | | autotaxin; reduces LPA levels both in plasma and | |
| | | | at the site of inflammation. | |
| G211- | 429.52 | Glucagon-like | Glucagon-like peptide 1 receptor inhibitor | PLpro(8.75); |
| 0145 | | peptide 1 | | nsp15(6.71) |
| | | receptor | | |
| K279- | 450.52 | ATP- | ATP-dependent Clp protease proteolytic subunit | PLpro(8.75); |
| 1256 | | dependent Clp | inhibitor | X |
| | | protease | | Domain(10.57) |
| | | proteolytic | | |
| | | subunit | | |
| K405- | 347.47 | Microtubule- | Microtubule-associated protein tau inhibitor | PLpro(8.72) |
| 3134 | | associated | | |
| | | protein tau | | |
| E216- | 354.41 | Microtubule- | Microtubule-associated protein tau inhibitor | PLpro(8.7) |
| 4969 | | associated | | |
| | | protein tau | | |
| C547- | 380.45 | Nonstructural | Nonstructural protein 1 inhibitor | PLpro(8.68) |
| 0142 | | protein 1 | | |
| C880- | 484.99 | Cellular tumor | Cellular tumor antigen p53 inhibitor | PLpro(8.65) |
| 2612 | | antigen p53 | | |
| E542- | 440.59 | DNA | DNA polymerase beta inhibitor | PLpro(8.63) |
| 1696 | | polymerase | | |
| | | beta | | |
| T7210 | 443.2 | Endogenous | Guanosine 5′-diphosphate as Potential Iron | PLpro(8.62) |
| | | Metabolite | Mobilizer, Preventing the Hepcidin-Ferroportin | |
| | | | Interaction and Modulating the Interleukin-6/Stat- | |
| | | | 3 Pathway. | |
| T5330 | 556.29 | GABA | Fluralaner is an isoxazoline ectoparasiticide. It | PLpro(8.61) |
| | | | potently and selectively inhibits binding of the | |
| | | Receptor | GABA receptor channel blocker EBOB to | |
| | | | housefly head membranes (IC50: 455 pM). | |
| 8009- | 435.47 | Androgen | Androgen Receptor inhibitor | PLpro(8.57) |
| 8507 | | Receptor | | |
| T7509 | 383.4 | Adenosine | PD 117519 is an agonist of adenosine receptor | PLpro(8.54) |
| | | Receptor | | |
| TMO2681 | 284.22 | Others | Inhibition of inosine and alanine-induced | PLpro(8.54) |
| | | | germination of Bacillus anthracis Sterne spore | |
| | | | pre-incubated for 15 mins. | |
| 6623- | 419.51 | Cellular tumor | Cellular tumor antigen p53 inhibitor | PLpro(8.53) |
| 1226 | | antigen p53 | | |
| T0148L | 601.58 | Others | Folinic Acid, a reduced folic acid, is used in | RdRP(11.73) |
| | | | combination with other chemotherapeutics. | |
| T6676 | 529.45 | HCV Protease | Sofosbuvir is a uridine monophosphate analog | RdRP(11.49) |
| | | inhibitor | inhibitor of hepatitis C virus (HCV) polymerase | |
| | | | NS5B that is used as an antiviral agent in the | |
| | | | treatment of chronic hepatitis C. | |
| T4060 | 580.47 | DNA/RNA | Acelarin (NUC-1031) is a ProTide enhancement | RdRP(11.46) |
| | | Synthesis | and transformation of the nucleoside analog, | |
| | | inhibitor | gemcitabine. | |
| C241- | 583.07 | Thyroid | Thyroid hormone receptor beta-1 inhibitor | RdRP(11.29) |
| 1670 | | hormone | | |
| | | receptor beta-1 | | |
| T3893 | 756.71 | NF-κB | Forsythoside B binds to LPS and reduces the | RdRP(11.24) |
| | | inhibitor | biological activity of serum LPS, and inhibits | |
| | | | NF-κB activation. Forsythoside B inhibits the | |
| | | | inflammatory response and has antioxidant | |
| | | | properties. Potent neuroprotective effects with a | |
| | | | favorable therapeutic time-window, reduce of | |
| | | | cerebral ischemia and reperfusion injury degree, | |
| | | | attenuating blood-brain barrier (BBB) | |
| | | | breakdown; Rescued cardiac function from I/R | |
| | | | injury. Forsythoside B has antisepsis effect, is | |
| | | | mediated by decreasing local and systemic levels | |
| | | | of a wide spectrum of inflammatory mediators. | |
| T7086 | 530.62 | Others | TBTA is a tertiary amine with three 1, 2, 3- | RdRP(11.15) |
| | | | triazole groups. It complexes with, and stabilizes, | |
| | | | copper(I) to accelerate azide-alkyne | |
| | | | cycloadditions, as used in click chemistry. | |
| T3780 | 594.52 | Others | Oroxin B has antioxidant activity. | RdRP(10.9) |
| C700- | 508.64 | Ataxin-2 | Ataxin-2 inhibitor | RdRP(10.85) |
| 0693 | | | | |
| C880- | 486.62 | Ataxin-2 | Ataxin-2 inhibitor | RdRP(10.77) |
| 0271 | | | | |
| T5345 | 538.69 | Others | V-9302 (V9302) is a competitive antagonist of | RdRP(10.77) |
| | | | transmembrane glutamine flux that selectively | |
| | | | and potently targets the amino acid transporter | |
| | | | ASCT2 (IC50: 9.6 uM). | |
| T0672 | 446.52 | HMG-CoA | Pravastatin sodium, an HMG-CoA | RdRP(10.69); |
| | | Reductase | reductase inhibitor, inhibits sterol synthesis with | X |
| | | inhibitor | IC50 of 5.6 μM. | Domain(10.4) |
| T3670 | 624.59 | Others | Forsythoside A has antimicrobial, | RdRP(10.67) |
| | | inhibitor | anticomplementary, anti-inflammatory and | |
| | | | antiendotoxin activities. Forsythoside A inhibits | |
| | | | Infected cells was confirmed by infecting primary | |
| | | | chicken embryo kidney cells. | |
| T4255 | 543.98 | PAI-1 | TM5275 is an inhibitor of plasminogen activator | RdRP(10.53) |
| | | | inhibitor 1 (PAI-1). | |
| 6747- | 340.38 | Alpha- | Alpha-galactosidase A inhibitor | RdRP(10.42); |
| 0106 | | galactosidase | | nsp15(6.78) |
| | | A | | |
| E946- | 436.56 | Nuclear factor | Nuclear factor erythroid 2-related factor 2 | RdRP(10.38) |
| 0779 | | erythroid 2- | inhibitor | |
| | | related factor | | |
| | | 2 | | |
| D072- | 479.99 | Mothers | Mothers against decapentaplegic homolog 3 | RdRP(10.35) |
| 0556 | | against | inhibitor | |
| | | decapentaplegic | | |
| | | homolog 3 | | |
| 6286- | 400.53 | Inositol | Inositol monophosphatase 1 inhibitor | RdRP(10.34) |
| 0223 | | monophosphatase | | |
| | | 1 | | |
| C448- | 475.6 | HEK293 | HEK293 inhibitor | RdRP(10.32) |
| 1053 | | | | |
| T5416 | 517.53 | MMP | T-5224 is a transcription factor c-Fos/AP-1 | RdRP(10.29) |
| | | | inhibitor, which specifically inhibits the DNA | |
| | | | binding activity of c-Fos/c-Jun without affecting | |
| | | | other transcription factors. | |
| C636- | 449.53 | HEK293 | HEK293 inhibitor | RdRP(10.26); |
| 2422 | | | | nsp15(7.06) |
| C336- | 538.07 | Ferritin light | Ferritin light chain inhibitor | RdRP(10.23) |
| 0089 | | chain | | |
| T1938 | 416.83 | FLT inhibitor | FLT3-IN-2 is an FLT3 inhibitor (IC50 <1 μM). | RdRP(10.22) |
| T2132 | 421.96 | 5-HT | Buspirone is a 5HT(1A) receptor agonist, used to | RdRP(10.22) |
| | | Receptor | treat generalized anxiety disorder (GAD). | |
| | | antagonist; | | |
| | | Dopamine | | |
| | | Receptor | | |
| | | antagonist | | |
| T5847 | 446.9 | Prostaglandin | Cloprostenol sodium is a more water solute, | RdRP(10.21); |
| | | Receptor | crystalline form of cloprostenol than the free acid, | X |
| | | | is a synthetic analog of prostaglandin F2α. | Domain(11.32) |
| T3263 | 563.53 | Antibiotic | Cefminox Sodium is a broad-spectrum, | RdRP(10.2) |
| | | inhibitor | bactericidal cephalosporin antibiotic. It is | |
| | | | especially effective against Gram-negative and | |
| | | | anaerobic bacteria. | |
| 8539- | 463.92 | Mothers | Mothers against decapentaplegic homolog 3 | RdRP(10.18) |
| 0868 | | against | inhibitor | |
| | | decapentaplegic | | |
| | | homolog 3 | | |
| T5384 | 417.51 | CCR | RS 504393 is a highly selective CCR2 chemokine | RdRP(10.17) |
| | | | receptor antagonist (IC50s: 89 nM and >100 μM | |
| | | | for human recombinant CCR2 and CCR1). | |
| K935- | 517.03 | Prelamin-A/C | Prelamin-A/C inhibitor | RdRP(10.05) |
| 0047 | | | | |
| 8014- | 379.42 | Cellular tumor | Cellular tumor antigen p53 inhibitor | RdRP(10.04) |
| 1195 | | antigen p53 | | |
| T2375 | 434.89 | CCR inhibitor | BX471 is a potent, selective non-peptide CCR1 | RdRP(10.04) |
| | | | antagonist. | |
| E843- | 454.45 | ATP- | ATP-dependent Clp protease proteolytic subunit | RdRP(10.03) |
| 0272 | | dependent Clp | inhibitor | |
| | | protease | | |
| | | proteolytic | | |
| | | subunit | | |
| G768- | 504.62 | ATP- | ATP-dependent Clp protease proteolytic subunit | RdRP(10.03) |
| 1619 | | dependent Clp | inhibitor | |
| | | protease | | |
| | | proteolytic | | |
| | | subunit | | |
| T6179 | 564.44 | Antibacterial | Moxalactam sodium salt is an antibiotic | RdRP(10) |
| | | inhibitor | compound more effective against Escherichia coli | |
| | | | and Pseudomonas aeruginosathan cephalosporins. | |
| T5725 | 718.61 | Sirtuin | lithospermic acid B is a water-soluble antioxidant | X |
| | | | from Salvia extract. It plays significant role of | Domain(14.2) |
| | | | antioxidant effect; antiplatelet aggregation, | |
| | | | anticoagulant, and antithrombotic effect | |
| T0136 | 488.32 | Dopamine | Citicoline is an intermediate in the synthesis of | X |
| | | Receptor | phosphatidylcholine, a component of cell | Domain(12.27) |
| | | antagonist | membranes. Citicoline exerts neuroprotective | |
| | | | effects. | |
| 8015- | 457.92 | Menin/Histone- | Menin/Histone-lysine N-methyltransferase MLL | X |
| 9350 | | lysine N- | inhibitor | Domain(11.91) |
| | | methyltransfer | | |
| | | ase MLL | | |
| 8015- | 469.5 | Nuclear factor | Nuclear factor erythroid 2-related factor 2 | X |
| 7947 | | erythroid 2- | inhibitor | Domain(11.69) |
| | | related factor 2 | | |
| T0154 | 441.9 | Adrenergic | Nebivolol hydrochloride is a cardioselective | X |
| | | Receptor | ADRENERGIC BETA-1 RECEPTOR | Domai |
| | | antagonist | ANTAGONIST (beta-blocker) that functions as a | n(11.38); |
| | | | VASODILATOR through the endothelial L- | nsp15 (7.55) |
| | | | arginine/NITRIC OXIDE system. It is used to | |
| | | | manage HYPERTENSION and chronic HEART | |
| | | | FAILURE in elderly patients. | |
| T3409 | 640.6 | Others | Plantamajoside has anti-hepatotoxic, anti- | X |
| | | | inflammatory, antinociceptive activities, | Domain(11.21) |
| | | | improving sexual function and antioxidant | |
| | | | activity. | |
| C528- | 437.52 | Geminin | Geminin inhibitor | X |
| 0901 | | | | Domain(11.16) |
| K284- | 489.57 | Beta- | Beta-lactamase AmpC inhibitor | X |
| 3774 | | lactamase | | Domain(11.11) |
| | | AmpC | | |
| C527- | 442.49 | Beta- | Beta-lactamase AmpC inhibitor | X |
| 0061 | | lactamase | | Domain(11.01) |
| | | AmpC | | |
| G678- | 465.55 | Geminin | Geminin inhibitor | X |
| 0299 | | | | Domain(10.97) |
| C060- | 525.63 | Beta- | Beta-lactamase AmpC inhibitor | X |
| 0100 | | lactamase | | Domain(10.88) |
| | | AmpC | | |
| T3708 | 626.15 | STAT | BP-1-102 is an orally active, effective and | X |
| | | inhibitor | specific STAT3 inhibitor. BP-1-102 binds Stat3 | Domain(10.84) |
| | | | (Kd: 504 nM), then blocks Stat3- | |
| | | | phosphotyrosine (pTyr) peptide interactions and | |
| | | | Stat3 activation (4-6.8 μM), and selectively | |
| | | | inhibits migration, survival, growth, and | |
| | | | invasion of Stat3-dependent tumor cells. The BP- | |
| | | | 1-102-mediated inhibition of aberrantly active | |
| | | | Stat3 in tumor cells suppresses the expression of | |
| | | | c-Myc, Bcl-xL, Cyclin D1, Survivin, and | |
| | | | VEGF. | |
| 7582- | 407.56 | Beta- | Beta-lactamase AmpC inhibitor | X |
| 0307 | | lactamase | | Domain(10.83) |
| | | AmpC | | |
| C336- | 559.58 | Mothers | Mothers against decapentaplegic homolog 3 | X |
| 0168 | | against | inhibitor | Domain(10.76); |
| | | decapentaplegic | | nsp15 (6.88) |
| | | homolog 3 | | |
| T3671 | 578.52 | Others | Vitexin-2″-O-rhamnoside is a compound | X |
| | | | contributes to the protection against H₂O₂ - | Domain(10.53) |
| | | | mediated oxidative stress damage. | |
| G205- | 443.53 | ATP- | ATP-dependent Clp protease proteolytic subunit | X |
| 0745 | | dependent Clp | inhibitor | Domain(10.5) |
| | | protease | | |
| | | proteolytic | | |
| | | subunit | | |
| Y041- | 478.51 | Geminin | Geminin inhibitor | X |
| 2269 | | | | Domain(10.49) |
| 7680- | 466.58 | Histone | Histone acetyltransferase GCN5 inhibitor | X |
| 2228 | | acetyltransferase | | Domain(10.37) |
| | | GCN5 | | |
| C200- | 517.05 | FK506 | FK506 binding protein 12 inhibitor | X |
| 1101 | | binding | | Domain(10.37); |
| | | protein 12 | | nsp15 (7.22) |
| T7142 | 478.78 | Others | Cephalosporin C zinc salt was a potent inhibitor | X |
| | | | of SAMHD1 (IC50: 1.1 ± 0.1 μM), and this | Domain(10.36) |
| | | | inhibition was largely attributable to the presence | |
| | | | of zinc. | |
| G856- | 486.58 | Prelamin-A/C | Prelamin-A/C inhibitor | X |
| 4196 | | | | Domain(10.29) |
| T2402 | 458.93 | 5-HT | Tianeptine sodium is a selective serotonin | X |
| | | Receptor | reuptake enhancer (SSRE), used to treat major | Domain(10.26) |
| | | agonist | depressive episodes. | |
| C906- | 423.56 | Plasmodium | Plasmodium falciparum inhibitor | X |
| 0334 | | falciparum | | Domain(10.18); |
| | | | | nsp15 (6.84) |
| T2923 | 460.5 | PDE inhibitor | Apremilast (CC-10004) is a potent and orally | X |
| | | | active PDE4 (IC50 = 74 nM) with anti- | Domain(10.09) |
| | | | inflammation activities. | |
| Y050- | 406.46 | Ataxin-2 | Ataxin-2 inhibitor | X |
| 1938 | | | | Domain(10.09) |
| T3879 | 482.44 | Lipoxygenase | Silychristin is a plant growth regulator. | X |
| | | inhibitor | Silychristin is an anti-hepatotoxic agent. | Domain(10.05) |
| | | | Silychristin is the inhibitor of horseradish | |
| | | | peroxidases and lipoxygenase. | |
| T1227 | 510.47 | Antibiotic | Cepazine is a second generation oral | X |
| | | | cephalosporin antibiotic. | Domain(10.03) |
| K786- | 406.53 | Microtubule- | Microtubule-associated protein tau inhibitor | X |
| 6600 | | associated | | Domain(9.99) |
| | | protein tau | | |
| E587- | 465.62 | Mothers | Mothers against decapentaplegic homolog 3 | X |
| 0421 | | against | inhibitor | Domain(9.98) |
| | | decapentaplegic | | |
| | | homolog 3 | | |
| C651- | 386.41 | Lysosomal | Lysosomal alpha-glucosidase inhibitor | X |
| 0859 | | alpha- | | Domain(9.97) |
| | | glucosidase | | |
| T1066 | 395.43 | 5-HT | Ketanserin is a quinazoline derivative and | X |
| | | Receptor | serotonin (5-hydroxytryptamine, 5HT) receptor | Domain(9.92) |
| | | antagonist | subtype 2 (5-HTR2) antagonist with potential | |
| | | | antihypertensive and antiplatelet activities. | |
| | | | Following administration, ketanserin binds to | |
| | | | and inhibits the signaling mediated by 5-HTR2, | |
| | | | which inhibits serotonin-dependent | |
| | | | vasoconstriction and platelet activation. | |
| T1331 | 478.33 | Vitamin | Riboflavin phosphate sodium is a water-soluble, | X |
| | | inhibitor | essential micronutrient that is the principal | Domain(9.92) |
| | | | growth-promoting factor in naturally occurring | |
| | | | vitamin B complexes. | |
| E465- | 398.5 | Beta- | Beta-lactamase AmpC inhibitor | X |
| 0564 | | lactamase | | Domain(9.91) |
| | | AmpC | | |
| T7100 | 395.41 | CSF-1R | PLX-5622 is a highly selective brain penetrant | X |
| | | | and oral active CSF1R inhibitor. | Domain(9.9) |
| E986- | 477.52 | Nuclear factor | Nuclear factor erythroid 2-related factor 2 | X |
| 1019 | | erythroid 2- | inhibitor | Domain(9.87) |
| | | related factor | | |
| | | 2 | | |
| C700- | 441.94 | Histone | Histone acetyltransferase GCN5 inhibitor | X |
| 1423 | | acetyltransferase | | Domain(9.77) |
| | | GCN5 | | |
| C636- | 477.57 | Cellular tumor | Cellular tumor antigen p53 inhibitor | X |
| 1184 | | antigen p53 | | Domain(9.74) |
| E461- | 397.44 | Cellular tumor | Cellular tumor antigen p53 inhibitor | nsp15(8.58) |
| 0614 | | antigen p53 | | |
| E461- | 383.41 | Beta- | Beta-lactamase AmpC inhibitor | nsp15(8.25) |
| 0573 | | lactamase | | |
| | | AmpC | | |
| T5171 | 412.5 | Prostaglandin | Treprostinil is a potent DP1, IP and EP2 agonist | nsp15(8.17) |
| | | Receptor; VE | (EC50: 0.6/1.9/6.2 nM). | |
| | | GFR; c-RET | | |
| T1503 | 331.15 | Adrenergic | Esmolol is a cardioselective beta-blocker used in | nsp15(7.98) |
| | | Receptor | parenteral forms in the treatment of arrhythmias | |
| | | antagonist | and severe hypertension. Esmolol has not been | |
| | | | linked to instances of clinically apparent drug | |
| | | | induced liver injury. | |
| T3886 | 428.43 | P450 inhibitor | Rosavin has antidepressant and anxiolytic | nsp15(7.94) |
| | | | actions, helps balance all the neurotransmitters. | |
| G948- | 468.58 | Ferritin light | Ferritin light chain inhibitor | nsp15(7.83) |
| 4026 | | chain | | |
| T2911 | 804.88 | TLR inhibitor | A natural, noncaloric sweetener with a potency | nsp15(7.72) |
| | | | 300 times more than that of regular sucrose. | |
| | | | Exhibits transepithelial p-aminohippurate | |
| | | | transport via organic anion transport system | |
| | | | interference. | |
| C700- | 540.69 | Mothers | Mothers against decapentaplegic homolog 3 | nsp15(7.7) |
| 1219 | | against | inhibitor | |
| | | decapentaplegic | | |
| | | homolog 3 | | |
| Y031- | 378.43 | Geminin | Geminin inhibitor | nsp15(7.68) |
| T6085 | 465.6 | S1P Receptor | PF-543, a novel sphingosine-competitive | nsp15(7.66) |
| | | inhibitor | inhibitor of SphK1, inhibits SphK1 with IC50 | |
| | | | and Ki of 2.0 nM and 3.6 nM. | |
| Y031- | 378.43 | Nuclear | Nuclear receptor ROR-gamma inhibitor | nsp15(7.66) |
| 0429 | | receptor ROR- | | |
| | | gamma | | |
| T3899 | 478.45 | Others | Calceolarioside B displays inhibition of | nsp15(7.63) |
| | | | aromatase. Calceolarioside B displays inhibition | |
| | | | of human recombinant PKCalpha. | |
| C880- | 457.54 | Guanine | Guanine nucleotide-binding protein G(s), subunit | nsp15(7.59) |
| 0694 | | nucleotide- | alpha inhibitor | |
| | | binding | | |
| | | protein G(s), | | |
| | | subunit alpha | | |
| T6846 | 410.51 | TLR inhibitor | GS-9620 is an effective and specific orally active | nsp15(7.55) |
| | | | agonist of Toll-like receptor 7. | |
| D715- | 435.48 | Plasmodium | Plasmodium falciparum inhibitor | nsp15(7.54) |
| 0611 | | falciparum | | |
| D134- | 341.37 | Histone-lysine | Histone-lysine N-methyltransferase, H3 lysine-9 | nsp15(7.5) |
| 0324 | | N- | specific 3 inhibitor | |
| | | methyltransferase, | | |
| | | H3 lysine-9 | | |
| | | specific 3 | | |
| Y031- | 387.28 | Nonstructural | Nonstructural protein 1 inhibitor | nsp15(7.49) |
| 2258 | | protein 1 | | |
| T7413 | 316.48 | Histamine | JNJ-5207852 is a novel, non-imidazole histamine | nsp15(7.48) |
| | | Receptor | H3 receptor antagonist, with high affinity at the | |
| | | | rat (pKi = 8.9) and human (pKi = 9.24) H3 receptor. | |
| T0342 | 522.49 | Adrenergic | Carvedilol Phosphate is the phosphate salt form | nsp15(7.48) |
| | | Receptor | of carvedilol, a racemic mixture and adrenergic | |
| | | antagonist; Gap | blocking agent with antihypertensive activity and | |
| | | Junction | devoid of intrinsic sympathomimetic activity. | |
| | | Protein | | |
| | | inhibitor; Integrin | | |
| | | inhibitor; NADPH | | |
| | | inhibitor; Others | | |
| | | inhibitor; | | |
| | | Potassium Channel | | |
| | | inhibitor; | | |
| | | VEGFR | | |
| | | inhibitor; LDL | | |
| D389- | 473.54 | Nonstructural | Nonstructural protein 1 inhibitor | nsp15(7.47) |
| 0267 | | protein 1 | | |
| 6913- | 388.51 | Beta- | Beta-lactamase AmpC inhibitor | nsp15(7.46) |
| 0019 | | lactamase | | |
| | | AmpC | | |
| T6S0119 | 624.77 | Others | 1. Dauricine has pulmonary toxicity, can | nsp15(7.44) |
| | | | produce pulmonary injury in CD-1 mice by the | |
| | | | metabolism of Dauricine mediated by CYP3A. 2. | |
| | | | Dauricinec can pass the blood-brain barrier, and | |
| | | | that P-glycoprotein has an important role in the | |
| | | | transportation of Dauricine across the blood-brain | |
| | | | barrier. 3. Dauricine may has anti-tumor effect, | |
| | | | can inhibit tumor cells in urinary system and | |
| | | | colon cancer cell proliferation, invasion; induce | |
| | | | cell apoptosis by suppressing NF-kappaB activity | |
| | | | and the expression profile of its downstream | |
| | | | genes. | |
| T7183 | 407.43 | Adenosine | CPI-444 is an antagonist of the adenosine A2A | nsp15(7.44) |
| | | Receptor | receptor. It reduces tumor area in mouse model of | |
| | | | murine Her2/neu-expressing breast cancer. | |
| G008- | 499.63 | Glucagon-like | Glucagon-like peptide 1 receptor inhibitor | nsp15(7.42) |
| 5517 | | peptide 1 | | |
| | | receptor | | |
| T0179 | 522.57 | Others | Ticagrelor, produced by AstraZeneca, is an | nsp15(7.42) |
| | | antagonist; P2 | inhibitor of platelet aggregation. Unlike | |
| | | Receptor | clopidogrel, ticagrelor is not a prodrug required | |
| | | inhibitor; | metabolic activation. The drug was approved for | |
| | | P450 | use in the European Union by the European | |
| | | antagonist | Commission on Dec. 3, 2010, and by the | |
| | | | US FDA on Jul. 20, 2011. Its trade names are | |
| | | | Brilinta (US), Brilique(EU) and Possia(EU). | |
| E977- | 431.51 | Thyroid | Thyroid hormone receptor beta-1 inhibitor | nsp15(7.42) |
| 0894 | | hormone | | |
| | | receptor beta- | | |
| | | 1 | | |
| 6655- | 361.47 | Inositol | Inositol monophosphatase 1 inhibitor | nsp15(7.4) |
| 0470 | | monophosphatase | | |
| | | 1 | | |
| T4S0998 | 446.4 | Adrenergic | Trifolirhizin exerts varying degrees of inhibition | nsp15(7.34) |
| | | Receptor; TNF | on tyrosinase-dependent melanin biosynthesis, | |
| | | | and therefore, are candidates as skin-whitening | |
| | | | agents. Trifolirhizin possesses potential anti- | |
| | | | inflammatory and anti-cancer | |
| | | | activities. Trifolirhizin shows in vitro inhibitory | |
| | | | effects on the growth of human A2780 ovarian | |
| | | | and H23 lung cancer cells. Trifolirhizin inhibits | |
| | | | acetylcholine mediated airway smooth muscle | |
| | | | (ASM) contraction or directly relaxes pre- | |
| | | | contracted ASM independent of β 2 - | |
| | | | adrenoceptors. | |
| T5S1094 | 462.44 | Others | Forsythoside E is a natutal product isolated from | nsp15(7.33) |
| | | | fruits of forsythia suspensa. | |
| T4602 | 462.53 | Others | Hydrocortisone 21-hemisuccinate is a | nsp15(7.32) |
| | | | corticosteroid used as an analytical and | |
| | | | chromatography reagent. | |
| E977- | 415.52 | Nuclear factor | Nuclear factor erythroid 2-related factor 2 | nsp15(7.32) |
| 0921 | | erythroid 2- | inhibitor | |
| | | related factor 2 | | |
| G937- | 497.02 | Prelamin-A/C | Prelamin-A/C inhibitor | nsp15(7.31) |
| 2830 | | | | |
| F070- | 488.51 | Cellular tumor | Cellular tumor antigen p53 inhibitor | nsp15(7.29) |
| 0397 | | antigen p53 | | |
| T0812 | 295.81 | Adrenergic | Propranolol hydrochloride is a widely used non- | nsp15(7.27) |
| | | Receptor | cardioselective beta-adrenergic antagonist. | |
| | | antagonist | Propranolol has been used for MYOCARDIAL | |
| | | | INFARCTION; ARRHYTHMIA; | |
| | | | ANGINAPECTORIS; HYPERTENSION; | |
| | | | HYPERTHYROIDISM; MIGRAINE; | |
| | | | PHEOCHROMOCYTOMA; and ANXIETY but | |
| | | | adverse effects instigate replacement by newer | |
| | | | drugs. | |
| G357- | 463.52 | Glucagon-like | Glucagon-like peptide 1 receptor inhibitor | nsp15(7.27) |
| 1938 | | peptide 1 | | |
| | | receptor | | |
| 5228- | 388.49 | Plasmodium | Plasmodium falciparum inhibitor | nsp15(7.25) |
| 0298 | | falciparum | | |
| E545- | 362.43 | Nuclear factor | Nuclear factor erythroid 2-related factor 2 | nsp15(7.25) |
| 0290 | | erythroid 2- | inhibitor | |
| | | related factor | | |
| | | 2 | | |
| T1959 | 600.02 | Histone | BIX01294 is an inhibitor of G9a histone | nsp15(7.25) |
| | | Methyltransferase | methyltransferase.In a cell-free assay, the | |
| | | inhibitor | IC50 = 2.7 μM for G9a histone methyltransferase. | |
| C199- | 479.58 | Mothers | Mothers against decapentaplegic homolog 3 | nsp15(7.24) |
| 0140 | | against | inhibitor | |
| | | decapentaplegic | | |
| | | homolog 3 | | |
| N081- | 355.39 | Beta- | Beta-lactamase AmpC inhibitor | nsp15(7.22) |
| 0783 | | lactamase | | |
| | | AmpC | | |
| C258- | 487.63 | TAR DNA- | TAR DNA-binding protein 43 inhibitor | nsp15(7.21) |
| 0605 | | binding | | |
| | | protein 43 | | |
| C199- | 499.57 | Ataxin-2 | Ataxin-2 inhibitor | nsp15(7.2) |
| 0115 | | | | |
| D011- | 475.98 | Cellular tumor | Cellular tumor antigen p53 inhibitor | nsp15(7.2) |
| 0852 | | antigen p53 | | |
| 1349- | 342.42 | ATPase | ATPase family AAA domain-containing protein 5 | nsp15(7.2) |
| 0007 | | family AAA | inhibitor | |
| | | domain- | | |
| | | containing | | |
| | | protein 5 | | |
| 5782- | 403.46 | Microtubule- | Microtubule-associated protein tau inhibitor | nsp15(7.16) |
| 5442 | | associated | | |
| | | protein tau | | |
| C528- | 404.53 | Guanine | Guanine nucleotide-binding protein G(s), subunit | nsp15(7.16) |
| 1116 | | nucleotide- | alpha inhibitor | |
| | | binding | | |
| | | protein G(s), | | |
| | | subunit alpha | | |
| K405- | 357.46 | Microtubule- | Microtubule-associated protein tau inhibitor | nsp15(7.14) |
| 3034 | | associated | | |
| | | protein tau | | |
| T5S0733 | 538.5 | Others | Picroside III, an iridoid glucoside found in the | nsp15(7.14) |
| | | | root of Picrorhiza scrophulariiflora Pennell | |
| | | | (Scrophulariaceae), and its subtype Picroside II | |
| | | | has been demonstrated to reduce apoptosis in | |
| | | | neuronal cells and other cell types. | |
| T6018 | 636.99 | P-gp | Zosuquidar (LY335979) is a potent modulator of | nsp15(7.14) |
| | | modulator | P-glycoprotein-mediated multi-drug resistance | |
| | | | with Ki of 60 nM. Phase 3. | |
| T2018 | 418.49 | IL Receptor | Apilimod inhibits the production of IL-12 and IL- | nsp15(7.13) |
| | | inhibitor; PI3K | 23 and reduces dendritic cell infiltration in | |
| | | | psoriasis. | |
| G361- | 436.56 | Glucagon-like | Glucagon-like peptide 1 receptor inhibitor | nsp15(7.13) |
| 0500 | | peptide 1 | | |
| | | receptor | | |
| C450- | 473.6 | Nuclear factor | Nuclear factor erythroid 2-related factor 2 | nsp15(7.12) |
| 0646 | | erythroid 2- | inhibitor | |
| | | related factor | | |
| | | 2 | | |
| K786- | 458.56 | Cellular tumor | Cellular tumor antigen p53 inhibitor | nsp15(7.11) |
| 2099 | | antigen p53 | | |
| 8388- | 376.31 | Microtubule- | Microtubule-associated protein tau inhibitor | nsp15(7.1) |
| 0819 | | associated | | |
| | | protein tau | | |
| 3257- | 415.88 | Microtubule- | Microtubule-associated protein tau inhibitor | nsp15(7.1) |
| 2542 | | associated | | |
| | | protein tau | | |
| D236- | 276.34 | Guanine | Guanine nucleotide-binding protein G(s), subunit | nsp15(7.07) |
| 0021 | | nucleotide- | alpha inhibitor | |
| | | binding | | |
| | | protein G(s), | | |
| | | subunit alpha | | |
| T0228 | 624.59 | Akt inhibitor; | Methyl Hesperidin, a flavanone glycoside | nsp15(7.06) |
| | | PKC inhibitor | (flavonoid) (C28H34O15), is abundant in citrus | |
| | | | fruits. Its aglycone form is called hesperetin. | |
| E750- | 409.46 | Mothers | Mothers against decapentaplegic homolog 3 | nsp15(7.06) |
| 0072 | | against | inhibitor | |
| | | decapentaplegic | | |
| | | homolog 3 | | |
| K279- | 512.64 | Prelamin-A/C | Prelamin-A/C inhibitor | nsp15(7.05) |
| 0833 | | | | |
| T6633 | 427.54 | Calcium | Ranolazine is a calcium uptake inhibitor via the | nsp15(7.04) |
| | | Channel | sodium/calcium channel, used to treat chronic | |
| | | inhibitor | angina. It affects the sodium-dependent calcium | |
| | | | channels during myocardial ischemia in rabbits | |
| | | | by altering the intracellular sodium level. | |
| T5711 | 462.45 | Others | Methylnissolin-3-O-glucoside has anti- | nsp15(7.02) |
| | | | inflammatory effects, antioxidant activity. | |
| D389- | 411.46 | HEK293 | HEK293 inhibitor | nsp15(7.01) |
| 0835 | | | | |
| 8009- | 410.5 | Cannabinoid | Cannabinoid CB1 receptor inhibitor | nsp15(7.01) |
| 9265 | | CB1 receptor | | |
| C795- | 403.55 | Aberrant vpr | Aberrant vpr protein inhibitor | nsp15(7.01) |
| 0720 | | protein | | |
| C320- | 401.51 | Plasmodium | Plasmodium falciparum inhibitor | nsp15(7.01) |
| 0115 | | falciparum | | |
| T3540 | 325.36 | Gamma- | IMR-1A is the metabolite of IMR-1 which is a | nsp15(7) |
| | | secretase | novel class of Notch inhibitors targeting the | |
| | | inhibitor | transcriptional activation with IC50 of 6 μMol/L. | |
| E544- | 538.55 | Mothers | Mothers against decapentaplegic homolog 3 | nsp15(6.99) |
| 0037 | | against | inhibitor | |
| | | decapentaplegic | | |
| | | homolog 3 | | |
| T2306 | 433.57 | 5-HT | Brexpiprazole is a partial agonist of human 5- | nsp15(6.98) |
| | | Receptor | hydroxytryptamine (5-HT) 5-HT1A and | |
| | | agonist; | dopamine D2 receptors. | |
| | | Adrenergic | | |
| | | Receptor | | |
| | | antagonist; | | |
| | | Dopamine | | |
| | | Receptor | | |
| | | agonist | | |
| C796- | 495.02 | Survival | Survival motor neuron protein inhibitor | nsp15(6.98) |
| 1295 | | motor neuron | | |
| | | protein | | |
| T2491 | 485.58 | EGFR | AZ5104 is a potent EGFR inhibitor. | nsp15(6.98) |
| | | inhibitor | | |
| D364- | 491.61 | TAR DNA- | TAR DNA-binding protein 43 inhibitor | nsp15(6.97) |
| 2068 | | binding | | |
| | | protein 43 | | |
| T3S1149 | 532.67 | Others | Ganoderic acid G is a highly oxidized lanostane- | nsp15(6.96) |
| | | | type triterpenoid from the fungus ganoderma | |
| | | | lucidum. | |
| C848- | 428.59 | Glucagon-like | Glucagon-like peptide 1 receptor inhibitor | nsp15(6.95) |
| 0215 | | peptide 1 | | |
| | | receptor | | |
| T0696 | 392.49 | Adrenergic | Naftopidil (INN, marketed under the brand name | nsp15(6.94) |
| | | Receptor | Flivas), an antihypertensive medicine, is used as | |
| | | antagonist | a selective α1-adrenergic receptor antagonist or | |
| | | | α-blocker. | |
| G856- | 472.55 | Cellular tumor | Cellular tumor antigen p53 inhibitor | nsp15(6.94) |
| 5036 | | antigen p53 | | |
| 8211- | 367.88 | Glucagon-like | Glucagon-like peptide 1 receptor inhibitor | nsp15(6.92) |
| 0295 | | peptide 1 | | |
| | | receptor | | |
| T6078 | 542.03 | BTK; c-Kit; | Saracatinib (AZD0530) is an effective Src | nsp15(6.91) |
| | | EGFR; Src | inhibitor (IC50: 2.7 nM), and effective to Lck, | |
| | | | Fyn, Lyn, Blk, Fgr and c-Yes. | |
| 5678- | 442.9 | Bromodomain | Bromodomain adjacent to zinc finger domain | nsp15(6.9) |
| 0009 | | adjacent to | protein 2B inhibitor | |
| | | zinc finger | | |
| | | domain | | |
| | | protein 2B | | |
| G408- | 382.44 | Cellular tumor | Cellular tumor antigen p53 inhibitor | nsp15(6.9) |
| 1998 | | antigen p53 | | |
| T2320 | 392.49 | Adrenergic | Indacaterol (Onbrez; Arcapta) is a β2-Adrenergic | nsp15(6.88) |
| | | Receptor | Agonist. The mechanism of action of indacaterol | |
| | | agonist | is as an Adrenergic beta2-Agonist. | |
| 4341- | 466.54 | Nuclear | Nuclear receptor ROR-gamma inhibitor | nsp15(6.88) |
| 0035 | | receptor ROR- | | |
| | | gamma | | |
| G071- | 538.6 | Nuclear factor | Nuclear factor erythroid 2-related factor 2 | nsp15(6.87) |
| 0411 | | erythroid 2- | inhibitor | |
| | | related factor | | |
| | | 2 | | |
| T4302 | 394.53 | Wnt/beta- | iCRT3 is a Wnt and β-catenin-responsive | nsp15(6.86) |
| | | catenin | transcription inhibitor. | |
| T6091 | 417.5 | c-Kit | CP-673451 is a specific inhibitor of PDGFRα/β | nsp15(6.86) |
| | | inhibitor; | (IC50: 10/1 nM) with antiangiogenic and | |
| | | PDGFR | antitumor activity and the selectivity is higher | |
| | | inhibitor; | 450-fold than other angiogenic receptors. | |
| | | VEGFR | | |
| | | inhibitor | | |
| 6872- | 434.5 | Geminin | Geminin inhibitor | nsp15(6.85) |
| 0294 | | | | |
| G361- | 408.51 | Beta- | Beta-lactamase AmpC inhibitor | nsp15(6.84) |
| 0499 | | lactamase | | |
| | | AmpC | | |
| E570- | 421.56 | Plasmodium | Plasmodium falciparum inhibitor | nsp15(6.84) |
| 2192 | | falciparum | | |
| K788- | 437.54 | Prelamin-A/C | Prelamin-A/C inhibitor | nsp15(6.84) |
| 6635 | | | | |
| T4050 | 461.61 | ROR agonist | GSK2981278 is a highly potent and selective | nsp15(6.83) |
| | | | inverse agonist of retinoic acid receptor-related | |
| | | | orphan receptor gamma (ROR gamma). | |
| D345- | 355.44 | FK506 | FK506 binding protein 12 inhibitor | nsp15(6.82) |
| 0032 | | binding | | |
| | | protein 12 | | |
| D072- | 476 | Histone | Histone acetyltransferase GCN5 inhibitor | nsp15(6.82) |
| 1432 | | acetyltransferase | | |
| | | GCN5 | | |
| 2191- | 412.45 | Geminin | Geminin inhibitor | nsp15(6.81) |
| 2709 | | | | |
| T3274 | 438.13 | c-Met/HGFR | S49076 is a novel, potent inhibitor of MET, | nsp15(6.81) |
| | | inhibitor; | AXL/MER, and FGFR1/2/3, blocking cellular | |
| | | FGFR | phosphorylation of MET, AXL, and FGFRs. | |
| | | inhibitor; | | |
| | | TAM | | |
| | | Receptor | | |
| | | inhibitor | | |
| C276- | 371.44 | Nonstructural | Nonstructural protein 1 inhibitor | nsp15(6.81) |
| 0156 | | protein 1 | | |
| C891- | 441.51 | Ataxin-2 | Ataxin-2 inhibitor | nsp15(6.81) |
| 1711 | | | | |
| G678- | 482 | Prelamin-A/C | Prelamin-A/C inhibitor | nsp15(6.8) |
| 0075 | | | | |
| K784- | 424.52 | Plasmodium | Plasmodium falciparum inhibitor | nsp15(6.79) |
| 6292 | | falciparum | | |
| 8104- | 435.98 | FK506 | FK506 binding protein 12 inhibitor | nsp15(6.78) |
| 06075 | | binding | | |
| T1533 | 390.82 | Others | Valganciclovir Hydrochloride is a hydrochloride | nsp15(6.77) |
| | | | salt form of valganciclovir, a prodrug form of | |
| | | | ganciclovir, a nucleoside analog of 2′- | |
| | | | deoxyguanosine, with antiviral activity. After | |
| | | | phosphorylation, valganciclovir is incorporated | |
| | | | into DNA, resulting in inhibition of viral DNA | |
| | | | polymerase, and viral replication. | |
| K786- | 497.62 | Beta- | Beta-lactamase AmpC inhibitor | nsp15(6.77) |
| 7206 | | lactamase | | |
| | | AmpC | | |
| G503- | 426.56 | Prelamin-A/C | Prelamin-A/C inhibitor | nsp15(6.76) |
| 0141 | | | | |
| C651- | 436.47 | DNA | DNA polymerase iota inhibitor | nsp15(6.76) |
| 0863 | | polymerase | | |
| | | iota | | |
| 1630- | 412.53 | Thyroid | Thyroid stimulating hormone receptor inhibitor | nsp15(6.75) |
| 1629 | | stimulating | | |
| | | hormone | | |
| | | receptor | | |
| T2795 | 457.43 | Others | Amygdalin has antifibrotic, antitumor, anti- | nsp15(6.75) |
| | | | inflammatory and analgesic effects, amygdalin | |
| | | | joint HSYA could inhibit the degeneration of the | |
| | | | endplate chondrocytes derived from intervertebral | |
| | | | discs of rats induced by IL-1beta and better than | |
| | | | the single use of Amygdalin or HSYA. | |
| | | | Amygdalin induces apoptotic cell death in human | |
| | | | DU145 and LNCaP prostate cancer cells by | |
| | | | caspase-3 activation through down-regulation of | |
| | | | Bcl-2 and up-regulation of Bax. | |
| K279- | 545.71 | HEK293 | HEK293 inhibitor | nsp15(6.74) |
| 1018 | | | | |
| T0990 | 379.43 | Dopamine | Droperidol is a Dopamine-2 Receptor Antagonist. | nsp15(6.74) |
| | | Receptor | The mechanism of action of droperidol is as a | |
| | | antagonist | Dopamine D2 Antagonist. | |
| C919- | 445.57 | Cellular tumor | Cellular tumor antigen p53 inhibitor | nsp15(6.74) |
| 0592 | | antigen p53 | | |
| T0216 | 463.43 | 5-HT | Eletriptan hydrobromide is an orally active | nsp15(6.73) |
| | | Receptor | agonist with specific affinity for the 5- | |
| | | agonist | hydroxytriptamine1B/1D receptor. | |
| C328- | 392.46 | Guanine | Guanine nucleotide-binding protein G(s), subunit | nsp15(6.73) |
| 0251 | | nucleotide- | alpha inhibitor | |
| | | binding | | |
| | | protein G(s), | | |
| | | subunit alpha | | |
| T2899 | 418.39 | Others | Liquiritin (LIQ) is a main component among the | nsp15(6.72) |
| | | | licorice flavonoids, and possesses anti- | |
| | | | inflammatory and anti-cancer abilities. | |
| 8011- | 348.42 | TAR DNA- | TAR DNA-binding protein 43 inhibitor | nsp15(6.72) |
| 7222 | | binding | | |
| | | protein 43 | | |
| K786- | 407.52 | Huntingtin | Huntingtin inhibitor | nsp15(6.71) |
| 6703 | | | | |
| T6853 | 380.48 | Histone | GSK591, Alternative Names are EPZ015866, | nsp15(6.71) |
| | | Methyltransferase | GSK3203591, is a potent selective inhibitor of | |
| | | inhibitor | the arginine methyltransferase PRMT5 (IC50 = 11 nM) | |
| K221- | 513 | Microtubule- | Microtubule-associated protein tau inhibitor | nsp15(6.71) |
| 2578 | | associated | | |
| | | protein tau | | |
| D345- | 424.37 | Prelamin-A/C | Prelamin-A/C inhibitor | nsp15(6.7) |
| 0054 | | | | |
| K906- | 500.65 | Neuropeptide | Neuropeptide S receptor inhibitor | nsp15(6.7) |
| 2514 | | S receptor | | |
| T6S0444 | 494.45 | MMP | Salvianolic acid A could protect the blood brain | nsp15(6.7) |
| | | | barrier through matrix metallopeptidase 9 (MMP- | |
| | | | 9) inhibition and anti-inflammation. | |
| K784- | 454.57 | Beta- | Beta-lactamase AmpC inhibitor | nsp15(6.7) |
| 6600 | | lactamase | | |
| | | AmpC | | |
| G242- | 389.52 | Geminin | Geminin inhibitor | nsp15(6.69) |
| 0650 | | | | |
| K279- | 481.02 | Microtubule- | Microtubule-associated protein tau inhibitor | nsp15(6.69) |
| 1177 | | associated | | |
| | | protein tau | | |
| E750- | 461.48 | Histone | Histone acetyltransferase GCN5 inhibitor | nsp15(6.69) |
| 0832 | | acetyltransferase | | |
| | | GCN5 | | |
| G069- | 457.55 | Nuclear factor | Nuclear factor erythroid 2-related factor 2 | nsp15(6.68) |
| 0064 | | erythroid 2- | inhibitor | |
| | | related factor | | |
| | | 2 | | |

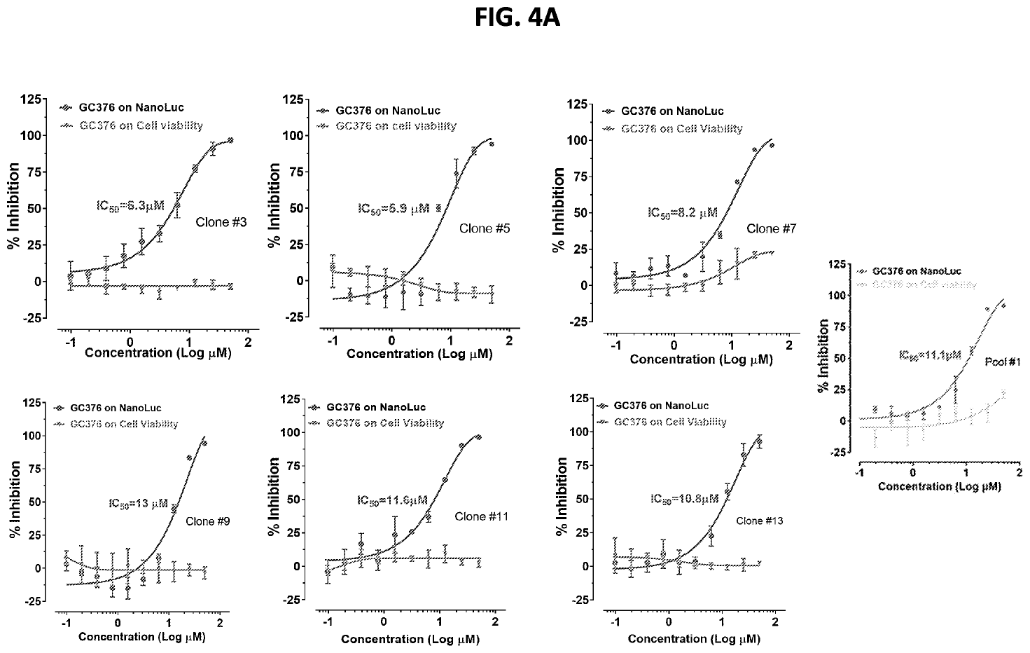

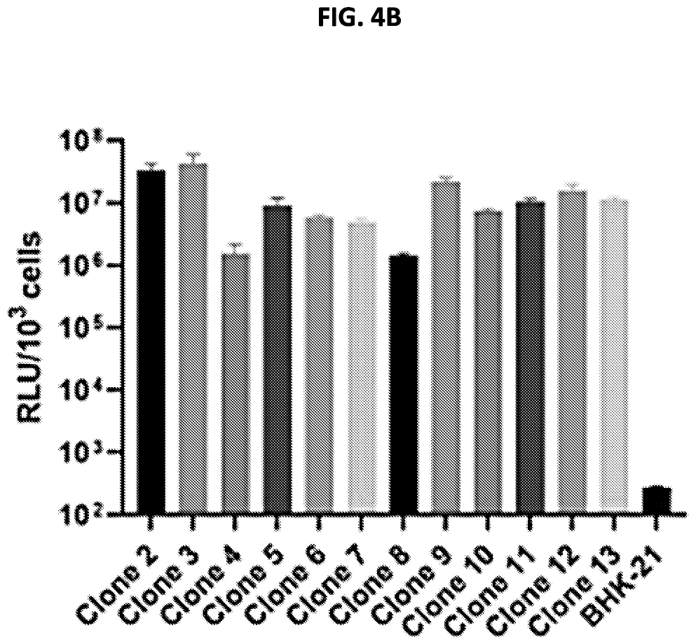

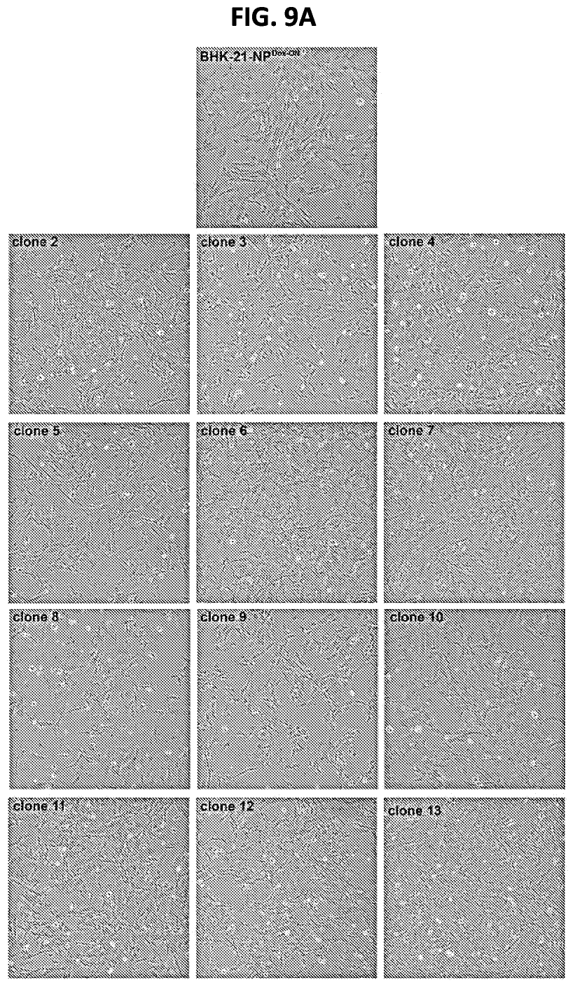

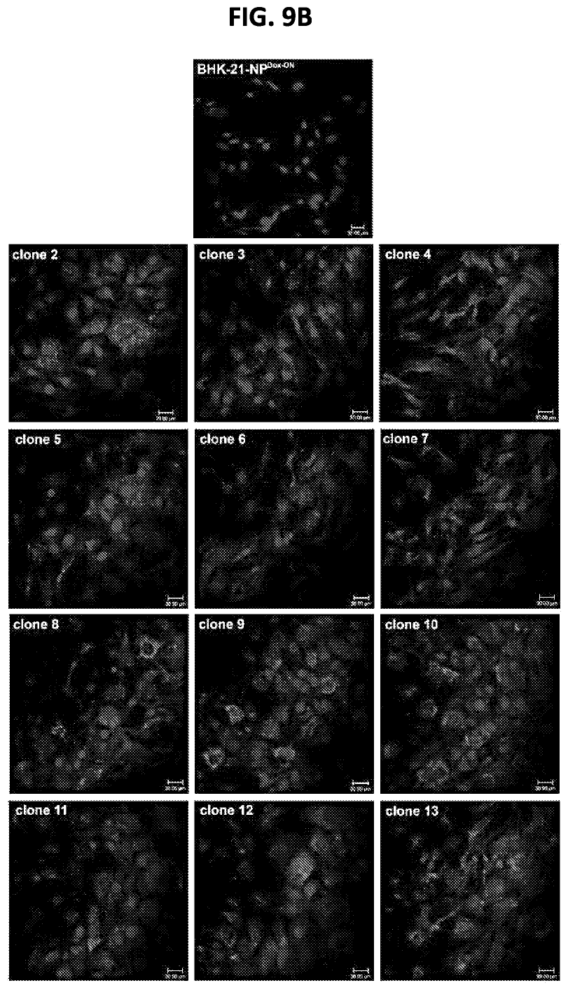

The IC₅₀ of these compounds are summarized in Table 6. All three compounds displayed cell type-specific activity against SARS-CoV-2, although others have also observed cell type-specific inhibition of SARS-CoV-2 by repurposed drugs (Dittmar et al., Cell Rep 35:108959 (2021)). To explore the robustness of replicon cells for drug screen, remdesivir was added to Pool #1 cells on different days following G418 withdrawal. Cells were subsequently incubated for 2-7 days before nanoluciferase was quantified. Little difference was observed in terms of the inhibitory strength of remdesivir when added between 2 and 9 days following G418 withdrawal (FIG. 9). By contrast, the optimal duration of remdesivir treatment in the cell culture was 3-6 days. Clonal variability was evaluated in response to drug treatment. Stable replicon cell clone #3, #5, #7, #9, #11 and #13 (SEQ ID NOs: 2, 4, 6, 8, 10, and 12) were tested for responses to GC376 treatment. Acquired IC₅₀ values ranged from 5.9 μM to 13 μM mean=9.3 μM, standard deviation=2.9 μM), indicating that all clones are suitable for testing drug efficacy (FIG. 4A). All 12 clones have been maintained for more than 20 passages under G418 without losing the expression of nano luciferase (FIG. 4B).

TABLE 6
Cell type-specific activity against SARS-CoV-2 by compound.

| | BHK-21 Replicon Cells¹ | | | A549-hACE2² | | | Calu-3³ |
|---|---|---|---|---|---|---|---|
| Compound | EC₅₀ | CC₅₀ | SI | EC₅₀ | CC₅₀ | SI | EC₅₀ |
| Darapladib | 7.0 ± 0.5 | 11.2 ± 2 | 1.6 | 0.8 ± 0.2 | 17.4 ± 8.1 | 21.8 | 0.9 ± 0.2 |
| Genz-123346 | 4.0 ± 1.8 | 26.3 ± 4.8 | 6.6 | 1.6 ± 0.5 | 99.7 ± 3.4 | 62.3 | 11.1 ± 3.6 |
| JNJ-5207852 | 8.1 ± 1.2 | 69.3 ± 39.6 | 8.6 | 3.1 ± 3 | >100 | >33 | 1.5 ± 48.5 |
| GC376 | 0.9 ± 0.2 | 25 ± 3.3 | 27.8 | 0.65 ± 0.2 | >50 | >77 | 0.2 ± 0.7 |
| Remdesivir | 1.6 ± 0.1 | 70.7 ± 11.6 | 44.2 | <0.1 | >50 | >500 | 0.2 ± 0.6 |

| | Calu-3³ | | Caco-2³ | | | Predicted |
|---|---|---|---|---|---|---|
| Compound | CC₅₀ | SI | EC₅₀ | CC₅₀ | SI | target⁴ |
| Darapladib | 7.6 ± 0.7 | 8.4 | 3.2 ± 2.8 | 9.4 ± 1.1 | 2.9 | 3CLpro |
| Genz-123346 | 33.3 ± 4.5 | 3 | 2 ± 3.6 | 77.8 ± 9 | 38.9 | Nsp16 |
| JNJ-5207852 | >100 | >66.7 | 1.0 ± 0.9 | >100 | >100 | Nsp15 |
| GC376 | >50 | >250 | 1.4 ± 0.8 | >50 | >36 | 3CLpro |
| Remdesivir | >50 | >250 | <0.1 | >50 | >500 | Nsp12 |
| | | | | | | (RdRP) |

¹Assays were performed in BHK-21 Pool #1 cells harboring SARS-CoV-2-Rep-NanoLuc-Neo-Nsp1^{K164A/H165A}.
²Assays were performed using live SARS-CoV-2 carrying a nanoluciferase and a firefly luciferase reporter.
³Assays were performed using live SARS-CoV-2 carrying a nanoluciferase reporter.
All values in M standard deviation from at least 3 biological or technical replicates.

In view of the many possible embodiments to which the principles of the disclosure may be applied, it should be recognized that the illustrated embodiments are only examples of the invention and should not be taken as limiting the scope of the invention. Rather, the scope of the invention is defined by the following claims. We therefore claim as our invention all that comes within the scope and spirit of these claims.

1. An isolated non-native coronavirus genome, comprising:
genetically inactivated spike (S), envelope (E), and membrane (M) genes;
a reporter gene;
a marker gene; and
a non-structural protein 1 (Nsp1) gene encoding (a) K164A and H165A substitutions, (b) N128S and K129E substitutions, or (c) R124S and K125E substitutions.

2. The isolated non-native coronavirus genome of claim 1, wherein the Nsp1 gene K164A substitution is encoded by guanine, cytosine, and cytosine residues at nucleotides 490, 491, and 492 of Nsp1, respectively, and the H165A substitution is encoded by guanine, cytosine, and cytosine residues at nucleotides 493, 494, and 495 of Nsp1, respectively.

3. The isolated non-native coronavirus genome of claim 1, wherein the genetically inactivated S, E, and M genes comprise one or more inactivating nucleotide mutations, insertions, or deletions.

4. The isolated non-native coronavirus genome of claim 1, wherein the non-native coronavirus genome further comprises a genetically inactivated nucleocapsid (NP) gene.

5. (canceled)

6. The isolated non-native coronavirus genome of claim 1, further comprising:
a non-structural protein 4 (Nsp4) gene encoding a R401S substitution;
a non-structural protein 10 (Nsp10) gene encoding a T111I substitution; or
both substitutions.

7. The isolated non-native coronavirus genome of claim 1, wherein the non-native coronavirus genome is an RNA molecule.

8. The isolated non-native coronavirus genome of claim 1, wherein the marker gene is a selectable marker gene.

9.-11. (canceled)

12. The isolated non-native coronavirus genome of claim 1, wherein the reporter gene encodes a fluorescent or bioluminescent protein.

13. (canceled)

14. The isolated non-native coronavirus genome of claim 1, wherein the isolated non-native coronavirus genome is a non-native betacoronavirus genome.

15. The isolated non-native coronavirus genome of claim 14, wherein the isolated non-native betacoronavirus genome is a non-native SARS-CoV genome, a non-native SARS-CoV-2 genome, or a non-native MERS-CoV genome.

16. (canceled)

17. The isolated non-native coronavirus genome of claim 1, comprising an isolated nucleic acid molecule comprising at least 80%, at least 85%, at least 90%, at least 92%, at least 95%, at least 96%, at least 97%, at least 98%, at least 99%, or 100% sequence identity to SEQ ID NO: 2, 3, 4, 5, 6, 7, 8, 9, 10, 11, 12, 13, or 1.

18. (canceled)

19. The isolated non-native coronavirus genome of claim 1, wherein the isolated non-native coronavirus genome is at least 20,000 kb, at least 24,000 kb, or 20,000 kb-30,000 kb.

20.-21. (canceled)

22. The isolated non-native coronavirus genome of claim 1, wherein the isolated non-native coronavirus genome is lyophilized.

23. A composition comprising:
the isolated non-native coronavirus genome of claim 1; and
a pharmaceutically acceptable carrier.

24. An isolated host cell comprising the isolated non-native coronavirus genome of claim 1.

25. The isolated host cell of claim 24, wherein the isolated non-native coronavirus genome is introduced into the cell using electroporation, liposome-mediated transfection, non-liposomal transfection, dendrimer-based transfection, particle bombardment, or microinjection.

26. The isolated host cell of claim 24, wherein the host cell is a mammalian cell.

27. The isolated host cell of claim 26, wherein the mammalian cell is a baby hamster kidney cell.

28. The isolated host cell of claim 27, wherein the baby hamster kidney cell is a BHK-21 cell.

29. The isolated host cell of claim 28, wherein the BHK-21 cell is the cell deposited as ATCC #______.

30. The isolated host cell of claim 24, wherein the host cell is a stable cell clone.

31. The isolated host cell of claim 24, wherein the isolated non-native coronavirus genome autonomously replicates in the host cell.

32. A composition, comprising:
the isolated host cell of claim 24; and
a culture medium, DMSO, or both.

33. A method of identifying an anti-viral compound, comprising:
contacting the isolated host cell of claim 24 with one or more compounds;
determining a level of expression of the reporter gene in the contacted cells; and
comparing the level of expression of the reporter gene in the contacted cells to a control; wherein reduced expression of the reporter gene in the contacted cells relative to the control indicates the compound is an anti-viral compound.

34.-39. (canceled)

40. A kit, comprising:
the isolated non-native coronavirus genome of claim 1; and
one or more of an antibiotic, transfection reagents, and culture media.

41. (canceled)
